(19) United States
(12) Patent Application Publication
(10) Pub. No.: US 2004/0005559 A1
(43) Pub. Date: Jan. 8, 2004

(54) Markers of neuronal differentiation and morphogenesis

(76) Inventors: Jeanne F. Loring, Foster City, CA (US); Matthew R. Kaser, Castro Valley, CA (US)

Correspondence Address: INCYTE CORPORATION (formerly known as Incyte, Genomics, Inc.), 3160 PORTER DRIVE, PALO ALTO, CA, 94304, US

(21) Appl. No.: 10/062,674

(22) Filed: Jan. 30, 2002

Related U.S. Application Data
(63) Continuation-in-part of application No. 09/625,102, filed on Jul. 24, 2000, now abandoned.

(51) Int. Cl. C12Q 1/68; C07H 21/04

(52) U.S. Cl. 435/6; 536/24.3

(57) ABSTRACT

The invention provides cDNAs that are diagnostic of and participate in neuronal differentiation and morphogenesis, proteins encoded by the cDNAs and agonists, antagonists, and antibodies that specifically bind the protein. The invention also provides compositions containing cDNAs, proteins, or antibodies and methods for their use diagnostically and therapeutically.

This application is a continuation-in-part of U.S. Ser. No. 09/625,102, filed Jul. 24, 2000.

### FIELD OF THE INVENTION

The invention relates to a combination comprising a plurality of cDNAs which are markers of neuronal differentiation and morphogenesis and may be used in the diagnosis, prognosis, treatment, and evaluation of therapies for conditions, diseases, disorders, and syndromes associated with neuronal differentiation and morphogenesis.

### BACKGROUND OF THE INVENTION

The hippocampus is part of the brain's limbic system which controls emotional behavior, motivational drive, and other physiological functions. The limbic system includes the limbic cortex, hippocampus, amygdala, hypothalamus, and anterior thalamus. The hippocampus plays an important role in learning and memory. Stimulation of the hippocampus can cause behavioral responses including rage, passivity, and excess sex drive. Weak electrical stimulation can cause hippocampal seizures. Individuals who lose hippocampal function retain memory for events that occurred prior to the loss, but only have immediate memory for all events after the loss (anterograde amnesia). Numerous studies of the effects of ablation of the hippocampus of rodent, primate, and other non-human species have been reported. Morphological changes in the hippocampus, including cell loss, are associated with epilepsy, schizophrenia, Alzheimer's disease, and certain amnesiac or memory problems or syndromes (Jack (1994) Epilepsia 35:S21-S29). Research with animal models show that glucocorticoids secreted during periods of stress can damage the hippocampus and impair the ability of hippocampal neurons to survive neurological insults (Sapolsky (1993) Behav Brain Res 57:175-182). Sustained hypersecretion of glucocorticoids from the adrenal cortex may damage the hippocampus; for example, hippocampal atrophy has been reported in patients with Cushing's syndrome.

Other disturbances to the environmental milieu of the brain may also contribute to changes in the phenotype of neurons, glia, oligodendrocytes, and microglia. Examples of cellular factors which have been shown to affect the morphology of cultured neuronal and glial cells include cytokines, growth factors, cellular and serum proteases, and steroid hormones. Bacterial toxins which induce cytokines may also contribute, either directly, or indirectly to changes in neural cell phenotype (reviewed by Reichlin (1998) Ann NY Acad Sci 840:301-316).

During normal brain development, most neurons undergo mitosis during the prenatal and the initial postnatal periods. Until as recently as 1998, it was thought that cells increased in size, but not in number, in most parts of the mature brain. Both Gray and Sundstrom (1998; Brain Res 790:52-59) and Kempermann et al. (1998; Curr Biol 8:939-942) have reported that neuronal cell proliferation can occur in the dentate gyrus of adult rodent and primate hippocampus.

A number of proteins are expressed specifically in neuronal cells. For example, amyloid precursor protein (APP) is a component of a complex protein network that may function in axonal elongation, dendritic arborisation and neuronal cell migration. Neuronal protein 3.1 is expressed at high levels in the embryonic brain and in the adult cerebellum, hippocampus and olfactory bulb. Hox 1.3 is expressed in fully differentiated neurons including the Purkinje neurons of the cerebellum, the pyramidal and dentate neurons of the hippocampus, and the motor neurons of the spinal cord (De Strooper et al. (2000) J Cell Sci 113:1857-1870; Studler et al. (1993) Eur J Neurosci 5:614-623; and Odenwald et al. (1987) Genes Dev 1:482-96).

Totipotent embryonic stem (ES) cells isolated from mammalian embryos retain the potential to reform an embryo. When ES cells are replaced inside a carrier embryo, they resume normal development and contribute to all tissues of the live-born animal. ES cells are the preferred cells used in the creation of experimental knockout rodent strains. Recently, the role of the LIM homeobox gene, Lhx5, was reported to control hippocampal morphogenesis and neuronal differentiation using a knockout mouse model (Zhao et al. (1999) Science 284:1155-1158).

Mouse ES cells have also been used to study the differentiation of various cell and tissues types in vitro; these include neural cells, hematopoietic lineages, and cardiomyocytes (Bain et al. (1995) Dev Biol 168:342-357; Wiles and Keller (1991) Development 111:259-267; and Klug et al. (1996) J Clin Invest 98:216-224). Recent developments in technical capabilities now demonstrate that ES cells derived from human blastocysts may also be manipulated in vitro to differentiate into eight separate cell lineages including endoderm, mesoderm, and ectodermal cell types (Thomson (1998) Science 282:1145-1147).

In early experiments, it was reported that differentiated neuronal and glial cells derived from retinoic acid-treated mouse ES cells were positive for the neuron-, astrocyte- and oligodendrocyte-specific antigens, MAP2, GFAP, and O4, respectively. Some neuronal cells were also positive for acetylcholinesterase activity or glutamic acid decarboxylase expression (Fraichard et al. (1995) J Cell Sci 108:3181-3188). More recently, additional markers of neuronal differentiation were identified in retinoic acid-treated mouse ES cells including Pax-6, Islet-½, Lim-3, HB-9, Phox2b, and EN1 (Renoncourt et al. (1998) Mech Dev 79:185-197). Other cellular markers undoubtedly are present during neuronal differentiation, but their identity is as yet unknown. Furthermore, developmental signaling and growth factor pathways are shared by both neuronal and thymic cells which suggests that there are many common pathways both during development and following tissue maturation. It is even likely that the blood-brain barrier evolved in order to prevent inappropriate neuronal dismorphology induced by serum-, leukocyte-, or lymphocyte-derived factors.

Identification of additional neuronal markers, in particular those which may regulate or modulate neuronal differentiation and morphogenesis, would enable the production of neuronal cells in culture. Such cultures are useful in transplants and in the development of gene therapies. In addition, detection of changes in activity of such markers is useful in early diagnosis and treatment of disorders associated with neuronal differentiation and morphogenesis.

### SUMMARY OF THE INVENTION

The invention provides a combination comprising a plurality of cDNAs wherein the cDNAs are SEQ ID NOs: 1-2217 of the Sequence Listing which are differentially expressed in neuronal differentiation and morphogenesis and complements of SEQ ID NOs: 1-2217. The invention also provides a combination comprising a plurality of mouse cDNAs, wherein the cDNAs are SEQ ID NOs: 1-1365 and the complements thereof. The invention additionally provides a combination comprising a plurality of mouse cDNAs, wherein the cDNAs are SEQ ID NOs: 1366-2217 and the complements thereof. In one embodiment, mouse cDNAs having the nucleic acid sequences of SEQ ID NOs: 1-102 are increased greater than 5-fold, and the mouse cDNAs having the nucleic acid sequences of SEQ ID NOs: 1303-1365 are decreased greater than 5-fold. In another embodiment, mouse cDNAs having the nucleic acid sequences of SEQ ID NOs: 1-19 are increased greater than 10-fold, and the human cDNAs having the nucleic acid sequences of SEQ ID NOs: 1353-1365 are decreased greater than 10-fold In one aspect, the human cDNA is a homolog of a mouse cDNA whose expression is increased greater than 5-fold and has a nucleic acid sequence selected from SEQ ID NOs: 1366, 1367, 1369, 1377, 1379, 1396, 1417, 1424, 1429, 1434, 1450, 1468, 1470, 1475, 1487, 1492, 1497, 1508, 1512, 1522, 1533, 1552, 1560, 1596, 1607, 1614, 1622, 1635, 1653, 1654, 1660, 1662, 1678, 1679, 1680, 1710, 1713, 1716, 1717, 1770, 1771, 1795, 1814, 1815, 1816, 1817, 1819, 1831, 1834, 1837, 1878, 1880, 1887, 1916, 1924, 1927, 1948, 1950, 1976, 2019, 2022, 2039, 2041, 2060, 2060, 2063, 2084, 2127, 2127, 2154, 2164, 2182, 2183, or is decreased greater than 5-fold and has a nucleic acid sequence selected from SEQ ID NOs: 1376, 1388, 1389, 1426, 1518, 1527, 1529, 1548, 1578, 1586, 1604, 1629, 1630, 1661, 1671, 1672, 1684, 1720, 1720, 1766, 1777, 1794, 1838, 1847, 1851, 1857, 1869, 1870, 1892, 1904, 1905, 1931, 1968, 1983, 2001, 2038, 2053, 2054, 2088, 2111, 2141, 2144, and 2165. In another aspect, the human cDNA is a homolog of a mouse cDNA whose expression is increased greater than 10-fold and has a nucleic acid sequence selected from SEQ ID NOs: 1379, 1607, 1653, 1654, 1678, 1679, 1713, 1771, 1976, 2022, 2127, 2164, or decreased greater than 10-fold and has a nucleic acid sequence selected from SEQ ID NOs: 1376, 1527, 1629, 1766, 1931, 2001, 2088, and 2144.

The invention provides a cDNA array comprising the combination and a substrate. The invention also provides a method of using an array to detect the presence of at least one nucleic acid in a sample comprising contacting the array with the sample containing nucleic acids under conditions which allow formation of hybridization complexes between the nucleic acids of the sample and the cDNAs of the array; and detecting any hybridization complexes so formed. In one aspect, the sample is from a subject with a disorder associated with neuronal differentiation and morphogenesis. In another aspect, hybridization complexes are compared with standards to diagnose a disorder associated with neuronal differentiation and morphogenesis. In one embodiment, the disorder is Alzheimer's disease; in another embodiment, the disorder is Parkinson's Disease; and in yet another embodiment, the disorder is cancer.

The invention also provides a method of using a cDNA array to screen a plurality of molecules or compounds to identify a molecule or compound which specifically binds to a cDNA of the array comprising contacting the array with a plurality of molecules or compounds under conditions to allow specific binding; and detecting specific binding, wherein a molecule or compound which specifically binds a cDNA of the array is identified. In one aspect, molecules or compounds are selected from DNA molecules, enhancers, mimetics, peptide nucleic acids, proteins, repressors, RNA molecules, and transcription factors. The invention further provides a method for obtaining a full length cDNA from a library of expressed or genomic nucleic acid sequences comprising arranging the library sequences on a substrate; hybridizing a cDNA selected from the Sequence Listing with the library sequences under conditions which allow hybridization; detecting hybridization between the cDNA and library sequence; isolating the library sequence, and sequencing the library sequence, and repeating these steps until a full length cDNA is obtained.

The invention provides an isolated human cDNA selected from SEQ ID NOs: 1367, 1372, 1376, 1377, 1383, 1389, 1390, 1397, 1400, 1401, 1406, 1408, 1412, 1413, 1415, 1416, 1420, 1438, 1444, 1460, 1462, 1467, 1470, 1476, 1477, 1485, 1489, 1491, 1497, 1500, 1501, 1503, 1512, 1517, 1541, 1544, 1551, 1553, 1555, 1557, 1565, 1566, 1572, 1576, 1577, 1579, 1580, 1581, 1582, 1583, 1585, 1588, 1598, 1599, 1601, 1603, 1609, 1610, 1612, 1620, 1625, 1632, 1634, 1642, 1643, 1649, 1658, 1686, 1690, 1691, 1693, 1708, 1721, 1727, 1730, 1731, 1738, 1746, 1748, 1756, 1757, 1786, 1789, 1797, 1823, 1825, 1829, 1830, 1847, 1852, 1864, 1866, 1875, 1882, 1887, 1889, 1894, 1895, 1901, 1938, 1949, 1954, 1960, 1961, 1965, 1966, 2002, 2019, 2043, 2046, 2054, 2066, 2067, 2069, 2071, 2072, 2078, 2079, 2100, 2105, 2121, 2125, 2139, 2151, 2154, 2157, 2170, 2171, 2183, 2184, 2186, 2187, 2208, and 2211 as presented in the Sequence Listing. The invention also provides an expression vector containing the cDNA, a host cell containing the expression vector, and a method for producing a protein comprising culturing the host cell under conditions for the expression of protein and recovering the protein from the host cell culture. The invention additionally provides a method for identifying a molecule or compound which specifically binds a cDNA comprising contacting a cDNA with a plurality of molecules and compounds under conditions which allow specific binding, and recovering the bound cDNA, wherein the molecule or compound is identified. The invention further provides a method for using a cDNA to purify a the molecule or compound from a sample comprising contacting the cDNA with a sample under conditions which allow specific binding between the cDNA and a molecule or compound, recovering the bound cDNA, and separating the cDNA wherein the molecule of compound is purified from the sample.

The invention provides a purified protein encoded by a cDNA of the invention. In one embodiment the protein is encoded by a cDNA selected from SEQ ID NOs: 1367, 1372, 1376, 1377, 1383, 1389, 1390, 1397, 1400, 1401, 1406, 1408, 1412, 1413, 1415, 1416, 1420, 1438, 1444, 1460, 1462, 1467, 1470, 1476, 1477, 1485, 1489, 1491, 1497, 1500, 1501, 1503, 1512, 1517, 1541, 1544, 1551, 1553, 1555, 1557, 1565, 1566, 1572, 1576, 1577, 1579, 1580, 1581, 1582, 1583, 1585, 1588, 1598, 1599, 1601, 1603, 1609, 1610, 1612, 1620, 1625, 1632, 1634, 1642, 1643, 1649, 1658, 1686, 1690, 1691, 1693, 1708, 1721, 1727, 1730, 1731, 1738, 1746, 1748, 1756, 1757, 1786, 1789, 1797, 1823, 1825, 1829, 1830, 1847, 1852, 1864, 1866, 1875, 1882, 1887, 1889, 1894, 1895, 1901, 1938, 1949, 1954, 1960, 1961, 1965, 1966, 2002, 2019, 2043, 2046, 2054, 2066, 2067, 2069, 2071, 2072, 2078, 2079, 2100, 2105, 2121, 2125, 2139, 2151, 2154, 2157, 2170, 2171, 2183, 2184, 2186, 2187, 2208, and 2211. The invention also provides a method for using a protein to screen a plurality of molecules or compounds to identify a molecule or compound which specifically binds the protein comprising combining the protein with a plurality of molecules or compounds under conditions to allow specific binding, recovering the bound protein, and separating the protein, thereby obtaining a molecule or compound which specifically binds the protein. In one embodiment, the molecules and compounds to be screened are selected from agonists, antagonists, antibodies, DNA molecules, small molecule drugs, immunoglobulins, inhibitors, mimetics, peptide nucleic acids, peptides, pharmaceutical agents, proteins, RNA molecules, ribozymes, and the like. In one aspect, the molecule or compound acts as a agonist, enhancer, inducer, or promoter in a test system. In another aspect, the molecule or compound acts as an antagonist or inhibitor in a test system.

The invention provides a method of using a protein to prepare and purify an antibody which specifically binds the protein comprising immunizing an animal with the protein under conditions to elicit an antibody response; isolating animal antibodies; contacting the protein with isolated antibodies under conditions to formation of an antibody:protein complex; and dissociating antibody from the complex, thereby obtaining purified antibody which specifically binds the protein. The invention also provides a method for using an antibody to detect expression of a protein which specifically binds the antibody in a sample, the method comprising combining the antibody with a sample under conditions which allow the formation of antibody:protein complexes; and detecting complex formation, wherein complex formation indicates expression of the protein which specifically binds the protein in the sample. In one embodiment, complex formation is compared with standards and is diagnostic for a disorder of neuronal differentiation and morphogenesis. The invention also provides a method for using an antibody to immunopurify a protein comprising attaching the antibody to a substrate; contacting the antibody with a sample containing the protein, thereby forming an antibody:protein complex; dissociating the antibody:protein complex; and collecting the purified protein. The invention further provides a composition comprising a cDNA, a protein, an antibody that specifically binds a protein or peptide, or a molecule or compound that specifically binds the cDNA, protein, or antibody and a pharmaceutical carrier for use in treating a disorder of neuronal differentiation or morphogenesis.

### DESCRIPTION OF THE COMPACT DISC-RECORDABLE (CD-R)

CD-R 1 is labeled: “pa26sqls.txt US, Copy 1,” was created on Jan. 30, 2002, and contains: pa26sqls.txt the Sequence Listing formatted in plain ASCII text. The file for the Sequence Listing is entitled pt01xxs1.txt, created on Jan. 30, 2002 and is 3.15 KB in size.

CD-R 2 is an exact copy of CD-R 1. CD-R 2 is labeled: “pa26sqls.txt US, Copy 2,” and was created on Jan. 30, 2002.

The CD-R labeled as: “pa26sqls.txt US, CRF,” contains the Sequence Listing formatted in plain ASCII text. The file for the Sequence Listing is entitled pa26sqls.txt was created on Jan. 30, 2002 and is 3.15 KB in size.

The content of the Sequence Listing named above and as described below, submitted in duplicate on two (2) CD-Rs (labeled “pa26sqls.txt US, Copy 1” and “pa26sqls.txt US, Copy 2”), and the CRF (labeled “pa26sqls.txt US, CRF”) containing the Sequence Listing, are incorporated by reference herein, in their entirety.

### DESCRIPTION OF THE TABLES

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of nucleic acid sequences obtained by sequencing clone inserts (isolates) of different cDNAs and identified using hybridization of samples against the cDNAs on a glass array. Each sequence is identified by a sequence identification number (SEQ ID NO) and by an Incyte ID number. The Incyte ID number represents a clone containing the cDNA insert. The Sequence Listing provides sequences of exemplary mouse cDNAs expressed in neuronal differentiation and morphogenesis, SEQ ID NOs: 1-1365, and their homologous human cDNAs, SEQ ID NOs: 1366-2217.

Table 1 shows the cDNAs, whose expression in retinoic acid-treated mouse ES cells is increased or decreased, SEQ ID NOs: 1-1365. Column 1 lists the SEQ ID NOs for each mouse cDNA. Column 2 shows the ratio of differential expression calculated from expression in the retinoic acid-treated sample divided by expression in the matched, untreated sample at the same time point in the experiment. Column 3 shows the description and IMAGE ID for each mouse cDNA.

Table 2 shows the cDNAs, whose expression in retinoic acid-treated mouse ES cells is increased or decreased and their human homologs. Column 1 lists the SEQ ID NO for each mouse cDNA. Column 2 lists the IMAGE clone ID of each mouse cDNA. Column 3 lists the GenBank ID for each mouse cDNA. Column 4 lists the Incyte template ID for the human cDNA that is identified as a homolog of the mouse cDNA. Column 5 lists the SEQ ID NO for the human cDNA. Column 6 lists the e values for the BLAST2 identified homolog of the Incyte human template. Column 7 shows the description of each Incyte human template. If no Incyte human template homolog was identified by BLAST2, no entry was made.

### DETAILED DESCRIPTION OF THE INVENTION

Before the nucleic acid sequences and methods are presented, it is to be understood that this invention is not limited to the particular machines, methods, and materials described. Although particular embodiments are described, machines, methods, and materials similar or equivalent to these embodiments may be used to practice the invention. The preferred machines, methods, and materials set forth are not intended to limit the scope of the invention which is limited only by the appended claims.

The singular forms “a”, “an”, and “the” include plural reference unless the context clearly dictates otherwise. All technical and scientific terms have the meanings commonly understood by one of ordinary skill in the art. All publications are incorporated by reference for the purpose of describing and disclosing the cell lines, vectors, and methodologies which are presented and which might be used in connection with the invention. Nothing in the specification is to be construed as an admission that the invention is not entitled to antedate such disclosure by virtue of prior invention.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody is of diagnostic or therapeutic interest. The arrangement of two to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

“Cancer” refers to adenocarcinoma, leukemia, lymphoma, melanoma, myeloma, sarcoma, teratocarcinoma, and, in particular, cancers or tumors of the adrenal gland, bladder, bone, bone marrow, brain, breast, cervix, colon, esophagus, gall bladder, ganglia, heart, kidney, liver, lung, muscle, ovary, pancreas, parathyroid, penis, prostate, salivary glands, skin, small intestine, spleen, stomach, testis, thymus, thyroid, and uterus.

A “combination” comprises at least two and up to about 2217 sequences as presented in the Sequence Listing.

“Complementary” describes the relationship between two single-stranded nucleotide sequences that anneal by base-pairing (5′-A-G-T-3′ pairs with its complement 3′-T-C-A-5′).

“cDNA” refers to an isolated polynucleotide, nucleic acid molecule, or any fragment or complement thereof. It may have originated recombinantly or synthetically, be double-stranded or single-stranded, and represent coding and noncoding 3′ or 5′ sequence, and generally it lacks introns. The cDNA may be combined with carbohydrate, lipids, protein or other materials to perform a particular activity such as diagnosis or to form a useful composition for mammalian therapy.

A “composition” refers to the cDNA and a labeling moiety; a purified protein and a pharmaceutical carrier or a heterologous, labeling or purification moiety; an antibody and a labeling moiety or pharmaceutical agent; and the like.

“Differential expression” refers to an increased or upregulated or a decreased or downregulated expression as detected by absence, presence, or at least two-fold change in the amount of transcribed messenger RNA, translated protein, or complexed antibody in a sample.

“Disorder” refers to conditions, diseases or syndromes associated with neuronal differentiation and morphogenesis including Alzheimer's disease, autism, cancer, ischemic cerebrovascular disease, epilepsy, Huntington's disease, Parkinson's disease, schizophrenia, and stroke.

An “expression profile” is a representation of gene expression in a sample. A nucleic acid expression profile is produced using sequencing, hybridization, or amplification technologies and mRNAs or cDNAs from a sample. A protein expression profile mirrors the nucleic acid expression profile and uses labeling moieties or antibodies to quantify the protein expression in a sample. The nucleic acids, proteins, or antibodies may be used in solution or attached to a substrate, and their detection is based on methods and labeling moieties well known in the art.

“Fragment” refers to at least 18 consecutive nucleotides of a cDNA of the Sequence Listing or its complement or to a portion of an antibody.

“Hybridization complex” refers to a complex between two nucleic acid molecules by virtue of the formation of hydrogen bonds between purines and pyrimidines.

“Identity” as applied to nucleic acid or protein sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997) Nucleic Acids Res 25:3389-3402). BLAST2 may be used in a standardized and reproducible way to insert gaps in one of the sequences in order to optimize alignment and to achieve a more meaningful comparison between them. Similarity is an analogous score, but it is calculated with conservative substitutions of residues taken into account; for example, substitution of a valine for a isoleucine or leucine.

“Isolated or purified” refers to a cDNA, protein, or antibody that is removed from its natural environment or from cell culture and that is separated from other components with which it is associated.

“Labeling moiety” refers to any reporter molecule whether a visible or radioactive label, stain or dye than can be attached to or incorporated into a cDNA or protein. Visible labels and dyes include but are not limited to anthocyanins, β glucuronidase, BIODIPY, Coomassie blue, Cy3 and Cy5, digoxigenin, FITC, green fluorescent protein, luciferase, spyro red, silver, and the like. Radioactive markers include radioactive forms of hydrogen, iodine, phosphorous, sulfur, and the like.

“Ligand” refers to any agent, molecule, or compound which will bind specifically to a complementary site on a cDNA, protein, or antibody of the invention. Such ligands stabilize, modulate, or disrupt the activity of cDNAs, proteins, or antibodies and may be composed of inorganic and/or organic substances including minerals, cofactors, nucleic acids, proteins, carbohydrates, fats, and lipids.

“Probe” refers to a cDNA that hybridizes to a nucleic acid molecule in a sample or on a substrate. A probe is used to detect, amplify, lengthen or quantify cDNAs, endogenous genes, or transcript mRNAs by employing conventional, molecular biology techniques. Probes are the reporter molecules of hybridization reactions including Southern, northern, in situ, dot blot, array, and like technologies.

“Protein” refers to a polypeptide or any portion thereof. A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR, Madison Wis.). An “oligopeptide” is an amino acid sequence from about five residues to about 15 residues that is used as part of a fusion protein to produce an antibody.

“Sample” may contain polynucleotides, proteins, agonists, antagonists, and antibodies and refers to a bodily fluid; a soluble fraction of a cell preparation or media in which cells were grown; a chromosome, an organelle, or membrane isolated or extracted from a cell; genomic DNA, RNA, or cDNA in solution or bound to a substrate; a cell; a tissue; a tissue print; a fingerprint, skin or hair; and the like.

“Specific binding” refers to the interaction between two molecules. In the case of a cDNA, specific binding may involve hydrogen bonding between sense and antisense strands or between one strand and a protein which affects its replication or transcription, intercalation of a molecule or compound into the major or minor groove of the DNA molecule, or interaction with at least one molecule which functions as a transcription factor, enhancer, repressor, and the like. In the case of a protein, specific binding may involve interactions with cDNAs, as described above or with molecules or compounds such as agonists, antibodies, antagonists, and the like. Specific binding is dependent upon the presence of structural features that allow chemical or molecular interactions between molecules.

“Substrate” refers to any rigid or semi-rigid support to which molecules or compounds are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to a nucleic acid molecule or protein whose sequence diverges from SEQ ID NOs: 1-2217 or their encoded proteins. Nucleic acid sequence divergence may result from natural or intentional mutational changes such as deletions, additions, and substitutions of one or more nucleotides or residues or from differential splicing. Intentional nucleotide mutations may be introduced recombinantly to accommodate differences in codon usage among species. Each deletion, addition, and substitution may occur alone, or in combination, one or more times in a given sequence.

The Invention

The invention provides a combination comprising a plurality of cDNAs which are markers of neuronal differentiation and morphogenesis. The cDNAs comprise at least a fragment of SEQ ID NOs: 1-2217 as presented in the Sequence Listing. The invention also provides a combination of mouse cDNAs, SEQ ID NOs: 1-1365, which were differentially expressed in mouse embryonic stem cells 16 days after treatment with RA. The invention provides a combination of mouse cDNAs whose expression is increased, SEQ ID NOs: 1-673, or decreased, SEQ ID NOs: 674-1365, following treatment. The invention also provides a combination of cDNAs whose expression is increased, SEQ ID NOs: 1-102, or decreased, SEQ ID NOs: 1303-1365, greater than 5-fold following treatment. The invention also provides a combination of cDNAs whose expression is increased, SEQ ID NOs: 1-19, or decreased, SEQ ID NOs: 1353-1365, greater than 10-fold following treatment. The invention also provides a combination comprising a plurality human cDNAs, SEQ ID NOs: 1366-2217, which have high sequence homology to the differentially expressed mouse sequences.

As shown in Table 1, sequences encoding developmental regulatory proteins e.g., growth factors (SEQ ID NO: 3, IMAGE ID 464598; SEQ ID NO: 20, IMAGE ID 367780), amyloid beta precursor protein (SEQ ID NO: 15, IMAGE ID 535652), and F-spondrin neural plate protein (SEQ ID NO: 69, IMAGE ID 762240), have expression levels increased by more than 5-fold compared with untreated ES cells. In addition, sequences encoding extracellular matrix proteins e.g., extracellular matrix protein 1 (SEQ ID NO: 5, IMAGE ID 874833), procollagen (SEQ ID NO: 6, IMAGE ID 420322), pleiotrophin (SEQ ID NO: 7, IMAGE ID 478168), and signal transduction proteins e.g., protein tyrosine kinase (SEQ ID NO: 42, IMAGE ID 401456), beta-2 microglobulin (SEQ ID NO: 90, IMAGE ID 572542) and calpain-like protease (SEQ ID NO: 34, IMAGE ID 747101), have expression levels increased by more than 5-fold in retinoic acid-treated cells compared with untreated cells. 102 IMAGE clones showed at least a 5-fold increase in expression when retinoic acid-treated ES cells were compared with untreated ES cells. Another 18 IMAGE unannotated clones showed at least a 5-fold increase in expression when retinoic acid-treated ES cells were compared with untreated ES cells These mouse sequences may be used as markers of neuronal differentiation and morphogenesis in mouse tissues or mammalian model systems.

As shown in Table 1, sequences encoding proteins promoting cell growth, including DNA replication proteins, e.g., replication factor C (SEQ ID NO: 1337, IMAGE ID 572970), mismatch repair protein MutS (SEQ ID NO: 1322, IMAGE ID 945359); proteins that regulate cell cycle progression e.g., cyclin B1 (SEQ ID NO: 1336, IMAGE ID 751977), cyclin E (SEQ ID NO: 1332, IMAGE ID 833511) and other growth-related proteins (SEQ ID NO: 1312, IMAGE ID 575434), showed at least a 5-fold decrease in expression when retinoic acid-treated ES cells were compared with untreated ES cells. Developmental regulatory proteins e.g., JM1 protein (SEQ ID NO: 1349, IMAGE ID 833919), F-Boc protein FBX115 ubiquitin ligase regulator (SEQ ID NO: 1361, IMAGE ID 833477); extracellular matrix proteins e.g., cadherin (SEQ ID NO: 1305, IMAGE ID 776133), spectrin (SEQ ID NO: 1344, IMAGE ID 403735), and signal transduction proteins e.g., asialoglycoprotein receptor 1 (SEQ ID NO: 1353, IMAGE ID 949512, KIAA0116 (SEQ ID NO: 1318, IMAGE ID 533085), showed at least a 5-fold decrease in expression when retinoic acid-treated ES cells were compared with untreated ES cells. 63 IMAGE clones showed at least a 5-fold decrease in expression when retinoic acid-treated ES cells were compared with untreated ES cells. Eight unannotated IMAGE clones showed at least a 5-fold decrease in expression when retinoic acid-treated ES cells were compared with untreated ES cells. These mouse sequences may be used as markers of neuronal differentiation and morphogenesis in mouse tissues or mammalian model systems.

As shown in Table 1, sequences encoding developmental regulatory proteins e.g., dual-specific nuclear matrix tyrosine protein kinase (SEQ ID NO: 502, IMAGE ID 354506), neuronal-associated protein (SEQ ID NO: 125, IMAGE ID 356215); cytoskeletal proteins e.g., myosin light chain (SEQ ID NO: 589, IMAGE ID 330370), actin-binding protein (SEQ ID NO: 174, IMAGE ID 408110); transcription factors e.g., KIAA0613 enigma/LIM2 (SEQ ID NO: 585, IMAGE ID 336726), GATA-GT2 Zn-finger (SEQ ID NO: 221, IMAGE ID 368189); extracellular matrix proteins e.g., GalBeatGalNAcAlpha sialyltransferase (SEQ ID NO: 361, IMAGE ID 481883), extracellular protein precursor (SEQ ID NO: 113, IMAGE ID 746798); and signal transduction proteins e.g., serine/threonine protein kinase 11 (SEQ ID NO: 161, IMAGE ID 388477) and Ftp-1 tyrosine phosphatase (SEQ ID NO: 206, IMAGE ID 400530), showed between a 2fold and a 5-fold increase in expression when retinoic acid-treated ES cells were compared with untreated ES cells. 165 unannotated IMAGE clones showed between a 2fold and a 5-fold increase in expression when retinoic acid-treated ES cells were compared with untreated ES cells. These mouse sequences may be used as markers of neuronal differentiation and morphogenesis in mouse tissues or mammalian model systems.

As shown in Table 1, sequences encoding proteins promoting cell growth e.g., Rac GTPase-activating protein (SEQ ID NO: 743, IMAGE ID 577676), regulator of chromosome condensation (SEQ ID NO: 1182, IMAGE ID 422799); developmental regulatory proteins e.g., KIAA0314 (SEQ ID NO: 1142, IMAGE ID 335017), neuronal pentraxin receptor (SEQ ID NO: 729, IMAGE ID 352795); transcription factors e.g., centaurin alpha (SEQ ID NO: 1186, IMAGE ID 437602), pirin NF-1 (SEQ ID NO: 1181, IMAGE ID 332396); extracellular matrix proteins e.g., Gar1 low molecular weight neurofilament APP-binding protein (SEQ ID NO: 997, IMAGE ID 475814), calpain p94 (SEQ ID NO: 1284, IMAGE ID 574491); and signal transduction proteins e.g., Nedd-4-like ubiquitin protein ligase regulator of Notch signaling pathway (SEQ ID NO: 832, IMAGE ID 354910, bisphosphate 3′nucleotidase 1 (SEQ ID NO: 957, IMAGE ID 439411), showed between a 2fold and a 5-fold decrease in expression when retinoic acid-treated ES cells were compared with untreated ES cells. 130 unannotated IMAGE clone sequences showed between a 2fold and a 5-fold decrease in expression when retinoic acid-treated ES cells were compared with untreated ES cells. These mouse sequences may be used as markers of neuronal differentiation and morphogenesis in mouse tissues or mammalian model systems.

The sequences of the mouse IMAGE clones were used to identify human templates from the LLFESEQ GOLD database (Incyte Genomics, Palo Alto Calif.) using BLAST2 with an e value cutoff of p<10⁻⁵. As shown in Table 2, SEQ ID NOs: 1366-2217 are considered to be human homologs of the mouse sequences SEQ ID NOs: 1-1365. As shown in Table 2, SEQ ID NOs: 1383, 1415, 1444, 1460, 1462, 1476, 1477, 1485, 1500, 1503, 1517, 1544, 1555, 1557, 1565, 1585, 1603, 1612, 1632, 1634, 1642, 1708, 1731, 1738, 1756, 1797, 1852, 1864, 1901, 1960, 2002, 2019, 2079, 2154, and 2208 are considered to be homologs of known human genes as described in the seventh column of Table 2. These human sequences are useful as markers of neuronal differentiation and morphogenesis in human tissues or tissue samples.

In addition, results from two samples that had been treated identically were compared to determine the range of normal variation of gene expression patterns between the samples. In one analysis, where two different samples were prepared from identically treated tissues, gene expression patterns of cDNAs which were increased or decreased not more than 1.7-fold were within the 95% confidence limits of a Poisson normal distribution. In a separate analysis, gene expression patterns of cDNAs which were increased or decreased more than 2-fold accounted for less than 5% of the total hybridizable sample nucleic acid molecules in two identically-treated tissue samples.

Refinement of treatment methods of cultured ES cells allowed the identification of a transitory period during which the phenotype of undifferentiated ES cells changed to a differentiated cell type comprising mainly phenotypically normal neurons (neuromorphogenesis). As shown in Table 1, the apparent neuronal phenotype of the differentiated ES cells was confirmed by increased levels of neuron-specific cellular markers, such as neuronal protein 3.1 (g1171753), amyloid β (A4) precursor protein (g112929), as well as two mouse ESTs, IMAGE: 463135 (homologous to rat IGF-II precursor) and IMAGE:639481 (homologous to rat IGF-binding protein 3). In contrast, the same analysis identified a number of proliferation- and retinoic acid-regulated transcripts whose levels were decreased including G2/mitotic-specific cyclin B1, uridine phosphorylase, and two mouse ESTs, IMAGE: 893933 and “EST highly similar to retinoic acid-regulated protein pH 34”, as shown in Table 1.

Mouse nucleic acids that are increased or decreased at least 2-fold at least once during the time course were identified. These molecules are SEQ ID NOs: 1-1365 provided in the Sequence Listing. Human nucleotide molecules highly homologous to the mouse nucleotide molecules were also identified. These molecules are SEQ ID NOs: 1366-2464 provided in the Sequence Listing.

Human neuronal cellular markers, such as neuronal DHP-sensitive, voltage-dependent, calcium channel alpha-2b subunit (SEQ ID NO: 1891); basic transcription factor 2 p44, neuronal apoptosis inhibitory protein (naip) and survival motor neuron protein (SEQ ID NO: 1719); calpain-like protease CANPX (SEQ ID NO: 1533); neurofilament-66 (SEQ ID NO: 1479); semaphorin III family homolog (SEQ ID NO: 1640); vascular endothelial cell growth factor 165 receptor/neuropilin (VEGF165) (SEQ ID NO: 1770); glia-derived nexin (SEQ ID NO: 2127); KIAA0287 (SEQ ID NO: 1976); retinal short-chain dehydrogenase/reductase (SEQ ID NO: 1607); P311 Human (3.1) (SEQ ID NO: 1678, 1679, 1680); amyloid A4(751) (SEQ ID NO: 1713); alpha-B-crystallin (SEQ ID NO: 1924); vascular endothelial cell growth factor 165 (SEQ ID NO: 1770); NMDA receptor glutamate-binding chain (SEQ ID NO: 1844, 2182); KIAA1114 (SEQ ID NO: 1948); calpain-like protease CANPX (SEQ ID NO: 1533, 1698); KIAA0913 (SEQ ID NO: 2175); neutral calponin (SEQ ID NO: 1443, 2156); KIAA0438 (SEQ ID NO: 1824); Hox2.2 gene for a homoeobox protein (SEQ ID NO: 2213); KIAA0992 (SEQ ID NO: 2162); peripheral myelin protein 22 (GAS3) (SEQ ID NO: 1997); KIAA1224 (SEQ ID NO: 2185); MAP kinase activated protein kinase (SEQ ID NO: 1772); KIAA0382 (SEQ ID NO: 1677); amyloid precursor protein homolog HSD-2 (SEQ ID NO: 1765); KIAA1075 (SEQ ID NO: 1422); KIAA0987 (SEQ ID NO: 1928); KIAA0251 (SEQ ID NO: 1763); KIAA1249 (SEQ ID NO: 1606); KIAA0280 (SEQ ID NO: 1460); KIAA1048 (SEQ ID NO: 1845); KIAA0964 (SEQ ID NO: 1387); human genomic DNA of 21q^{22.2} Down Syndrome region (SEQ ID NO: 1897); KIAA1094 (SEQ ID NO: 1855); tactile protein (SEQ ID NO: 1989); S100 alpha protein (SEQ ID NO: 1987); KIAA0623 (SEQ ID NO: 1547); KIAA0630 (SEQ ID NO: 1995); KIAA1008 (SEQ ID NO: 1696); KIAA1007 (SEQ ID NO: 1563); calpain-like protease (htra-3) (SEQ ID NO: 1698); KIAA0011 (SEQ ID NO: 1982); KIAA1080 (SEQ ID NO: 2052); KIAA0136 (SEQ ID NO: 1988); KIAA0518 (SEQ ID NO: 2012); KIAA0595 (SEQ ID NO: 1528); KIAA0281 (SEQ ID NO: 2163); KIAA0156 (SEQ ID NO: 1626); KIAA0560 (SEQ ID NO: 1478); KIAA0253 (SEQ ID NO: 1441); KIAA1177 (SEQ ID NO: 1667, 1668); KIAA1261 (SEQ ID NO: 1930); glutathione transferase (GSTA3) (SEQ ID NO: 2210); KIAA1259 (SEQ ID NO: 1873); KIAA0274 (SEQ ID NO: 2085); KIAA1089 (SEQ ID NO: 1832); chaperonin containing t-complex polypeptide 1 (SEQ ID NO: 1854); KIAA0778 (SEQ ID NO: 1464); KIAA0111 (SEQ ID NO: 2160); KIAA0589 (SEQ ID NO: 1996); KIAA0314 (SEQ ID NO: 1856); KIAA1273 (SEQ ID NO: 2009); glutamate dehydrogenase (SEQ ID NO: 1941); KIAA0956 (SEQ ID NO: 2023); KIAA0631 (SEQ ID NO: 1591, 2079); KIAA0432 (SEQ ID NO: 1865); KIAA0007 (SEQ ID NO: 1920); KIAA0888 (SEQ ID NO: 1527); KIAA0314 (SEQ ID NO: 1856); KIAA0020 (SEQ ID NO: 1749); KIAA0116 (SEQ ID NO: 1672); glutamine PRPP amidotransferase (GPAT) (SEQ ID NO: 1905); and KIAA0888 (SEQ ID NO: 1527) were also identified, as shown in Table 2.

As shown in Table 2, human cDNAs which are highly homologous to mouse sequences which co-expressed with known neuronal markers during neuronal differentiation and morphogenesis were identified. These sequences include SEQ ID NOs: 1367, 1372, 1376, 1377, 1389, 1390, 1400, 1406, 1408, 1412, 1413, 1420, 1438, 1467, 1470, 1489, 1491, 1497, 1501, 1512, 1541, 1551, 1553, 1566, 1576, 1577, 1579, 1580, 1581, 1582, 1583, 1588, 1598, 1599, 1609, 1620, 1625, 1643, 1658, 1686, 1690, 1691, 1721, 1727, 1730, 1746, 1748, 1757, 1786, 1789, 1823, 1825, 1830, 1847, 1866, 1875, 1887, 1894, 1949, 1954, 1965, 1966, 2043, 2046, 2054, 2066, 2067, 2069, 2071, 2072, 2078, 2100, 2121, 2125, 2139, 2151, 2157, 2170, 2171, 2183, 2184, 2186, and 2187.

The invention provides a specific and useful method for diagnosing a disease associated with differential gene expression during neuronal differentiation and morphogenesis. The cDNAs of the invention define a differential expression pattern against which to compare the expression pattern of biopsied and/or treated tissues. Experimentally, differential expression of the cDNAs can be evaluated by other methods including, but not limited to, differential display by spatial immobilization or by gel electrophoresis, genome mismatch scanning, representational discriminant analysis, clustering, transcript images, and other array technologies. These methods may be used alone or in combination to verify the differential expression patterns that characterize a particular tissue, disorder, or therapy.

Transcript images for SEQ ID NOs: 1376 and 2164 are shown in EXAMPLE X. These TIs validate the differential expression patterns revealed by the array data and show that SEQ ID NOs: 1376 and 2164, when used in a tissue specific and clinically relevant manner, are diagnostic for germinal tumors (teratocarcinoma and embryonal carcinoma) and meningioma, respectively. The cDNAs can also be used to evaluate the efficacy of a particular treatment administered to a subject with germinal tumor or meningioma, respectively.

cDNAs and Their Uses

The cDNAs of the invention can be genomic DNA, mRNA, or any RNA-like or DNA-like material such as peptide nucleic acids, branched DNAs and the like. cDNA probes can be sense or antisense strand. In one embodiment, cDNAs are plasmids, and the sequence of interest is the cDNA insert.

Nucleotide analogs can be incorporated into cDNAs by methods well known in the art. The only requirement is that the incorporated nucleotide analogs must base pair with adenine, cytosine, guanine, thymine, or uracil. For example, guanine can be substituted with hypoxanthine which base pairs with cytosine. However, these base pairs are less stable than those between guanine and cytosine. Alternatively, adenine can be substituted with 2,6-diaminopurine which can form stronger base pairs with thymidine than those between adenine and thymidine. Additionally, cDNAs can include nucleotides that have been derivatized chemically or enzymatically. Typical chemical modifications include derivatization with acyl, alkyl, aryl or amino groups.

cDNAs can be synthesized or immobilized on a substrate. Synthesis on the surface of a substrate may be accomplished using a chemical coupling procedure and a piezoelectric printing apparatus as described by Baldeschweiler et al. (PCT publication WO95/251116). Alternatively, the cDNAs can be synthesized on a substrate surface using a self-addressable electronic device that controls when reagents are added as described in U.S. Pat. No. 5,605,662; incorporated herein by reference. cDNAs can be immobilized by covalent means such as by chemical bonding procedures or TV irradiation. In one such method, a cDNA is bound to a glass surface which has been modified to contain epoxide or aldehyde groups. In another case, a cDNA probe is placed on a polylysine coated surface and then UV cross-linked as described by Shalon et al. (WO95/35505). In yet another method, a cDNA is actively transported from a solution to a given position on a substrate by electrical means (Heller, supra). Alternatively, cDNAs, clones, plasmids or cells can be arranged on a filter. In the latter case, cells are lysed, proteins and cellular components degraded, and the DNA is coupled to the filter by UV cross-ling.

Furthermore, cDNAs do not have to be directly bound to the substrate, but rather can be bound to the substrate through a linker group. The linker groups are typically about 6 to 50 atoms long to provide exposure of the attached probe. Preferred linker groups include ethylene glycol oligomers, diamines, diacids and the like. Reactive groups on the substrate surface react with a terminal group of the linker to bind the linker to the substrate. The other terminus of the linker is then bound to the cDNA.

cDNAs can be attached to a substrate by sequentially dispensing reagents for probe synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

The cDNAs may be used for a variety of purposes. For example, the combination of the invention may be used on an array. The array can be used in high-throughput methods such as for detecting a related cDNA or protein in a sample, screening a plurality of molecules or compounds to identify a ligand, or diagnosing a particular condition, disease, or disorder of neuronal differentiation and morphogenesis. Alternatively, a cDNA complementary to a given sequence of the sequence listing can inhibit or inactivate a therapeutically relevant and closely related gene.

When the combination of the invention is employed on an array, the cDNAs are organized in an ordered fashion so that each element is present at a specified location on the substrate. Because the cDNAs are at specified locations on the substrate, the hybridization patterns and intensities, which together create a unique expression profile, can be interpreted in terms of expression levels of particular genes and can be correlated with a particular metabolic process, condition, disorder, disease, stage of disease, or treatment.

The cDNAs themselves or fragments of SEQ ID NOs: 1-1365 are useful in hybridization or amplification technologies to identify changes in expression pattern of the same or similar sequences in model systems. The cDNAs or fragments of SEQ ID NOs: 1366-2217 are useful in hybridization or amplification technologies to identify changes in expression pattern of the same or similar sequences in normal versus diseased tissues.

Hybridization

The cDNAs, oligonucleotides, fragments, or complements thereof may be used in various hybridization technologies. The cDNAs may be naturally occurring, recombinant, or chemically synthesized; based on genomic or cDNA sequences; and labeled using a variety of reporter molecules by either PCR or enzymatic techniques. Commercial kits are available for labeling and cleanup of such cDNAs or probes. Radioactive (Amersham Pharmacia Biotech (APB), Piscataway N.J.), fluorescent (Qiagen Operon, Alameda Calif.), and chemiluminescent labeling (Promega, Madison Wis.), are well known in the art. Alternatively, a cDNA is cloned into a commercially available vector, and probes are produced by transcription. The probe is synthesized and labeled by addition of an appropriate polymerase, such as T7 or SP6 polymerase, and at least one labeled nucleotide.

A probe may be designed or derived from unique regions of the cDNA, such as the 3′ untranslated region or from a conserved motif, and used in protocols to identify naturally occurring molecules encoding the same protein, allelic variants, or related molecules. The probe may be DNA or RNA, is usually single stranded and should have at least 50% sequence identity to any of the nucleic acid sequences. The probe may comprise at least 18 contiguous nucleotides, or an oligonucleotide, of a cDNA. Such a probe may be used under hybridization or amplification conditions that allow binding only to an identical sequence or under conditions that allow binding to a related sequence with at least one nucleotide substitution or deletion. Discovery of related sequences may also be accomplished using a pool of degenerate probes and appropriate hybridization conditions. Generally, a probe for use in Southern or northern hybridizations may be from about 400 to about 4000 nucleotides long. Such probes may be single-stranded or double-stranded and may have high binding specificity in solution-based or substrate-based hybridizations.

The stringency of hybridization is determined by G+C content of the probe, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffets, such as 5× saline sodium citrate (SSC) with 1% sodium dodecyl sulfate (SDS) at 60 C., that permits the formation of a hybridization complex between nucleic acid sequences that contain some mismatches. Subsequent washes are performed with buffers such as 0.2×SSC with 0.1% SDS at either 45 C. (medium stringency) or 65-68 C. (high stringency). At high stringency, hybridization complexes will remain stable only where the cDNAs are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel et al. (1997; Short Protocols in Molecular Biology, John Wiley & Sons, New York N.Y., 6.11-6.19, 14.11-14.36, and A1-43).

Dot-blot, slot-blot, low density and high density arrays are prepared and analyzed using methods known in the art. Probes or array elements from about 18 consecutive nucleotides to about 5000 consecutive nucleotides are contemplated by the invention and used in array technologies. The preferred number of probes or array elements is at least about 40,000; a more preferred number is at least about 18,000; an even more preferred number is at least about 10,000; and a most preferred number is at least about 600 to about 800. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; PCT application WO95/11995; PCT application WO95/35505; U.S. Pat. No. 5,605,662; and U.S. Pat. No. 5,958,342.)

Screening Assays

A combination comprising a plurality of cDNAs or an isolated cDNA may be used to screen a library or a plurality of molecules or compounds for a ligand with specific binding affinity. The ligands may be DNA molecules, RNA molecules, peptide nucleic acids, mimetics, and proteins such as transcription factors, enhancers, repressors, and other proteins that regulate the activity, replication, transcription, or translation of nucleic acid molecules in the biological system. The assay involves combining a combination or a cDNA of the invention or complements thereof with the molecules or compounds under conditions that allow specific binding to at least one cDNA and detecting the bound cDNA to identify at least one ligand that specifically binds it.

In one embodiment, the cDNA of the invention may be incubated with a library of isolated and purified molecules or compounds and binding activity determined by methods well known in the art, e.g., a gel-retardation assay described in U.S. Pat. No. 6,010,849 or a commercially available reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

Protein Production and Uses

The cDNAs or their full length equivalents may be used to produce peptides or proteins using recombinant DNA technologies described herein and taught in Ausubel (supra; pp. 16.1-16.62). One of the advantages of producing proteins by these procedures is the ability to obtain an highly-enriched sample thereby maximizing production.

The invention also encompasses amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.) or RasMol software (University of Massachusetts, Amherst Mass.) may be used to help determine which and how many amino acid residues in a particular portion of the protein may be substituted, inserted, or deleted without abolishing biological or immunological activity.

Expression of Encoded Proteins

Expression of a particular cDNA may be accomplished by cloning the cDNA into an appropriate vector and transforming this vector into an appropriate host cell. The cloning vector used for the construction of the human and rat cDNA libraries may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into an appropriate host strain of E. coli. Induction of the isolated bacterial strain with isopropylthiogalactoside (IPTG) using standard methods will produce a fusion protein that contains an N terminal methionine, the first seven residues of β-galactosidase, about 15 residues of linker, and the protein encoded by the cDNA.

The cDNA may be shuttled into other vectors known to be useful for expression of protein in specific hosts. Oligonucleotides containing cloning sites and fragments of a cDNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together to produce a fusion protein which may be expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the protein and the purification domain may also be used to recover the protein.

Suitable expression hosts may include, but are not limited to, mammalian cells such as Chinese Hamster Ovary (CHO) and human 293 cells, insect cells such as Sf9 cells, yeast cells such as Saccharomyces cerevisiae, and bacteria such as E. coli. For each of these cell systems, a useful expression vector may also include an origin of replication and one or two selectable markers to allow selection in bacteria as well as in a transfected eukaryotic host. Vectors for use in eukaryotic expression hosts may require the addition of 3′poly(A) tail if the cDNA lacks poly(A).

Additionally, the vector may contain promoters or enhancers that increase gene expression. Most promoters are host specific, and they include MMTV, SV40 or metallothionein promoters for CHO cells; trp, lac, tac or T7 promoters for bacterial hosts; or alpha factor, alcohol oxidase or PGH promoters for yeast. Adenoviral vectors with enhancers such as the rous sarcoma virus (RSV) enhancer or retroviral vectors with promoters such as the long terminal repeat (LTR) promoter may be used to drive protein expression in mammalian cell lines. Once homogeneous cultures of recombinant cells are obtained, large quantities of a secreted soluble protein may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, proteins may be produced using solid-phase techniques (Stewart et al. (1969) Solid-Phase Peptide Synthesis, W H Freeman, San Francisco Calif.; Merrifield (1963) J Am Chem Soc 5:2149-2154), manually, or using machines such as the ABI 431A Peptide synthesizer (Applied Biosystems (ABI), Foster City Calif.). Proteins produced by any of the above methods may be used as pharmaceutical compositions to treat disorders associated with underexpression.

Derivative refers to a cDNA or a protein that has been subjected to a chemical modification. Illustrative of such modifications would be replacement of a hydrogen by, for example, an acetyl, acyl, alkyl, amino, formyl, or morpholino group. Derivative cDNAs may encode proteins that retain the essential biological characteristics (such as catalytic and regulatory domains) of naturally occurring proteins.

Post-translational modification of a protein may involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

Screening Assays

A protein encoded by the cDNA may be used to screen libraries or a plurality of molecules or compounds for a ligand with specific binding affinity or to purify a molecule or compound from a sample. The protein employed in such screening may be free in solution, affixed to an abiotic or biotic substrate, or located intracellularly. For example, viable or fixed prokaryotic host cells that are stably transformed with recombinant nucleic acids that have expressed and positioned a protein on their cell surface can be used in screening assays. The cells are screened against libraries or a plurality of ligands and the specificity of binding or formation of complexes between the expressed protein and the ligand may be measured. The ligands may be DNA, RNA, or peptide nucleic acid molecules, agonists, antagonists, antibodies, immunoglobulins, inhibitors, peptides, pharmaceutical agents, proteins, drugs, or any other test molecule or compound that specifically binds the protein. An exemplary assay involves combining the mammalian protein with molecules or compounds under conditions that allow specific binding and detecting the bound protein to identify at least one ligand that specifically binds the protein.

This invention also contemplates the use of competitive drug screening assays in which neutralizing antibodies specifically compete with a test compound capable of binding the protein. One method for high throughput screening using very small assay volumes and very small amounts of test compound is described in U.S. Pat. No. 5,876,946. Molecules or compounds identified by screening may be used in a mammalian model system to evaluate their toxicity, diagnostic, or therapeutic potential.

Purification of a Ligand

The protein may be used to purify a ligand from a sample. A method for using a mammalian protein to purify a ligand would involve combining the protein with a sample under conditions to allow specific binding, recovering the bound protein, and using an appropriate chaotropic agent to separate the protein from the purified ligand.

Production of Antibodies

A protein encoded by a cDNA of the invention may be used to produce specific antibodies. Antibodies may be produced using an oligopeptide or any antigenic portion of the protein. Methods for producing antibodies include: 1) injecting an animal (usually goats, rabbits, or mice) with the protein, or an oligopeptide, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the proteins of the invention are useful for the diagnosis of prepathologic disorders as well as the diagnosis of chronic or acute diseases characterized by abnormalities in the expression, amount, or distribution of the protein. A variety of protocols for competitive binding or immunoradiometric assays using either polyclonal or monoclonal antibodies specific for proteins are well known in the art. Immunoassays typically involve the formation of complexes between a protein and its specific binding molecule or compound and the measurement of complex formation. A two-site, monoclonal-based immunoassay utilizing monoclonal antibodies reactive to two noninterfering epitopes on a specific protein is preferred, but a competitive binding assay may also be employed.

Immunoassay procedures may be used to quantify expression of the protein in cell cultures, in subjects with a particular disorder or in model animal systems under various conditions. Increased or decreased production of proteins as monitored by immunoassay may contribute to knowledge of the cellular activities associated with developmental pathways, engineered conditions or diseases, or treatment efficacy. The quantity of a given protein in a given tissue may be determined by performing immunoassays on freeze-thawed detergent extracts of biological samples and comparing the slope of the binding curves to binding curves generated by purified protein.

Labeling of Molecules for Assay

A wide variety of reporter molecules and conjugation techniques are known by those skilled in the art and may be used in various cDNA, protein or antibody arrays or assays. Synthesis of labeled molecules may be achieved using Promega or APB kits for incorporation of a labeled nucleotide such as ³²P-dCTP, Cy3-dCTP or Cy5-dCTP or amino acid such as ³⁵S-methionine. cDNAs, proteins, or antibodies may be directly labeled with a reporter molecule by chemical conjugation to amines, thiols and other groups present in the molecules using reagents such as BIODIPY or FITC (Molecular Probes, Eugene Oreg.).

The proteins and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241.

Diagnostics

The cDNAs may be used to detect and quantify altered gene expression; absence, presence, or excess expression of mRNAs; or to monitor mRNA levels during therapeutic intervention. The nucleic acid molecules can be used in diagnosis, prognosis, treatment, and evaluation of therapies for diseases associated with neuronal differentiation and morphogenesis, such as Alzheimer's disease, autism, cancer, ischemic cerebrovascular disease, epilepsy, Huntington's disease, Parkinson's disease, schizophrenia, and stroke. These cDNAs can also be utilized as markers of treatment efficacy against the diseases noted above and other conditions and diseases over a period ranging from several days to months. The diagnostic assay may use hybridization or amplification technology to compare gene expression in a biological sample from a patient to standard samples in order to detect altered gene expression. Qualitative or quantitative methods for this comparison are well known in the art.

For example, the cDNA may be labeled by standard methods and added to a biological sample from a patient under conditions for the formation of hybridization complexes. After an incubation period, the sample is washed and the amount of label (or signal) associated with hybridization complexes, is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a substantially purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

Expression Profiles

An expression profile can be based on differential expression of nucleic acid or proteins as measured using a number of different experimental technologies. Experimentally, differential expression of the cDNAs can be evaluated by other methods including, but not limited to, differential display by spatial immobilization or by gel electrophoresis, genome mismatch scanning, representational discriminant analysis, clustering, transcript images, and other array technologies. These methods may be used alone or in combination to verify the differential expression patterns that characterize a particular tissue, disorder, or therapy. Similarly the differential expression of proteins can be evaluated using protein or antibody arrays, ELISAs, FACS, RIAs and the like.

Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, an array is employed to improve the treatment regimen. A dosage is established that causes a change in expression indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease or treatment of the condition, disorder or disease. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against antigenic determinant of a protein encoded by a cDNA of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the protein and its specific antibody and the measurement of such complexes. These and other assays are described in Pound (1998; Immunochemical Protocols, Humana Press, Totowa N.J.). The method may employ a two-site, monoclonal-based immunoassay utilizing monoclonal antibodies reactive to two non-interfering epitopes, or a competitive binding assay. (See, e.g., Coligan et al. (1997) Current Protocols in Immunology, Wiley-Interscience, New York N.Y.; Pound, supra)

Therapeutics

The cDNAs may be used in gene therapy. The cDNAs may be delivered to a particular target organ, tissue, or cell population such as cells of the nervous system. Expression of the protein encoded by the cDNA may correct a disease state associated with reduction or loss of endogenous target protein. cDNAs may be delivered to specific cells in vitro, and then the cells transferred surgically to the intended tissue or delivered by the circulatory system The cDNAs are delivered to cells or tissues using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; August et al. (1997) Gene Therapy (Advances in Pharmacology, Vol. 40), Academic Press, San Diego Calif.).

In addition, expression of a particular protein can be modulated through the specific binding of an antisense sequence to a nucleic acid which either encodes the protein or directs its expression. The antisense sequence can be DNA, RNA, or nucleic acid mimics and analogs. The modulated nucleic acid can be cellular mRNA and/or genomic DNA, and the binding of the antisense sequence can affect translation and/or transcription, respectively. Antisense sequences can be delivered intracellularly using viral vectors or non-viral vectors as described above (Weiss et al. (1999) Cell Mol Life Sci 55(3):334-358; Agrawal (1996) Antisense Therapeutics, Humana Press., Totowa N.J.).

Both the cDNAs and antisense sequences can be produced ex vivo by using any of the ABI nucleic acid synthesizers or other automated systems known in the art. cDNAs and antisense sequences can also be produced biologically by transforming an appropriate host cell with an expression vector containing the cDNA or any fragment or complement thereof.

Molecules which modulate the expression of a cDNA of the invention or activity of the encoded protein are useful as therapeutics for conditions and disorders associated with neuronal differentiation and morphogenesis. Such molecules include agonists which increase the expression or activity of the endogenous gene or encoded protein, respectively; or antagonists which decrease expression or activity of the gene or protein, respectively. In one aspect, an antibody which specifically binds the protein may be used directly as an antagonist or indirectly as a targeting or delivery mechanism for bringing a pharmaceutical agent to cells or tissues which express the protein.

Additionally, any of the proteins or their ligands, or complementary nucleic acids may be administered in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with neuronal differentiation and morphogenesis. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

Transgenic Animal Models

Transgenic rodents that overexpress or underexpress a gene of interest may be inbred and used to model human diseases or to test therapeutic or toxic agents. (See, e.g., U.S. Pat. No. 5,175,3 83 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodernal cell types that differentiate into, for example, neural cells, hematopoietic lineages, and cardiomyocytes.

Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines that lack a functional copy of the mammalian gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

As described herein, the uses of the cDNAs provided in the Sequence Listing and their encoded proteins are exemplary of known techniques and are not intended to reflect any limitation on their use in any technique that would be known to the person of average skill in the art. Furthermore, the cDNAs provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining or assembling full length cDNA sequences that will be known to those skilled in the art.

### EXAMPLES

I Construction of cDNA Libraries

RNA was purchased from Clontech Laboratories (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Invitrogen). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNase. For most libraries, poly(A) RNA was isolated using oligo d(T)-coupled paramagnetic particles (Promega), OLIGOTEX latex particles (Qiagen, Valencia Calif.), or an OLIGOTEX mRNA purification kit (Qiagen). Alternatively, poly(A) RNA was isolated directly from 5 tissue lysates using other kits, including the POLY(A)PURE mRNA purification kit (Ambion, Austin Tex.).

In some cases, Stratagene (La Jolla Calif.) was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen) using the recommended procedures or similar methods known in the art. (See Ausubel, supra, Units 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (APB) or preparative agarose gel electrophoresis. cDNAs were ligated into compatible restriction enzyme sites of the polylinker of the pBLUESCRIPT phagemid (Stratagene), pSPORT1 plasmid (Invitrogen), or pINCY plasmid (Incyte Genomics). Recombinant plasmids were transformed into XL 1-BLUE, XL1-BLUEMRF, or SOLR competent E. coli cells (Stratagene) or DH5α, DH10B, or ELECTROMAX DH10B competent E. coli cells (Invitrogen).

In some cases, libraries were superinfected with a 5× excess of the helper phage, M13K07, according to the method of Vieira et al. (1987; Methods Enzymol 153:3-11) and normalized or subtracted using a methodology adapted from Soares (1994; Proc Natl Acad Sci 91:9228-9232), Swaroop et al. (1991; Nucleic Acids Res 19:1954), and Bonaldo et al. (1996; Genome Res 6:791-806). The modified Soares normalization procedure was utilized to reduce the repetitive cloning of highly expressed high abundance cDNAs while maintaining the overall sequence complexity of the library. Modification included significantly longer hybridization times which allowed for increased gene discovery rates by biasing the normalized libraries toward those infrequently expressed low-abundance cDNAs which are poorly represented in a standard expression profile (Soares, supra).

II Isolation and Sequencing of cDNA Clones

Plasmids were recovered from host cells by in vivo excision using the UNIZAP vector system (Stratagene) or by cell lysis. Plasmids were purified using one of the following: the Magic or WIZARD MINIPREPS DNA purification system (Promega); the AGTC MINIPREP purification kit (Edge BioSystems, Gaithersburg Md.); the QIAWELL 8, QIAWELL 8 PLUS, or QIAWELL 8 ULTRA plasmid purification systems, or the REAL PREP 96 plasmid purification kit (Qiagen). Following precipitation, plasmids were resuspended in 0.1 ml of distilled water and stored, with or without lyophilization, at 4 C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes) and a FLUOROSKAN II fluorescence scanner (Labsystems Oy, Helsinki, Finland).

cDNA sequencing reactions were processed using standard methods or high-throughput instrumentation such as the ABI CATALYST 800 thermal cycler (ABI) or the DNA ENGINE thermal cycler (MJ Research, Watertown Mass.) in conjunction with the HYDRA microdispenser (Robbins Scientific, Sunnyvale Calif.) or the MICROLAB 2200 system (Hamilton, Reno Nev.). cDNA sequencing reactions were prepared using reagents provided by APB or supplied in sequencing kits such as the ABI PRISM BIGDYE cycle sequencing kit by ABI. Electrophoretic separation of cDNA sequencing reactions and detection of labeled nucleotides were carried out using the MEGABACE 1000 DNA sequencing system (APB); the ABI PRISM 373 or 377 sequencing system (ABI) in conjunction with standard ABI protocols and base calling software; or other sequence analysis systems known in the art. Reading frames within the cDNA sequences were identified using standard methods (reviewed in Ausubel, supra, Unit 7.7).

III Extension of cDNA Sequences

Nucleic acid sequences were extended using Incyte cDNA clones and oligonucleotide primers. One primer was synthesized to initiate 5′ extension of the known fragment, and the other, to initiate 3′ extension of the known fragment. The initial primers were designed using OLIGO software (Molecular Insights, Cascade Colo.), or another appropriate program, to be about 22 to 30 nucleotides in length, to have a GC content of about 50% or more, and to anneal to the target sequence at temperatures of about 68 C. to about 72 C. Any stretch of nucleotides which would result in hairpin structures and primer-primer dimerizations was avoided.

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (MJ Research). The reaction mix contained DNA template, 200 nmol of each primer, reaction buffer containing Mg²⁺, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B (Incyte Genomics): Step 1: 94 C., 3 min; Step 2: 94 C., 15 sec; Step 3: 60 C., 1 min; Step 4: 68 C., 2 min; Step 5: Steps 2, 3, and 4 repeated 20 times; Step 6: 68 C., 5 min; Step 7: storage at 4 C. In the alternative, the parameters for primer pair T7 and SK+ (Stratagene) were as follows: Step 1: 94 C., 3 min; Step 2: 94 C., 15 sec; Step 3: 57 C., 1 min; Step 4: 68 C., 2 min; Step 5: Steps 2, 3, and 4 repeated 20 times; Step 6: 68 C., 5 min; Step 7: storage at 4 C.

The concentration of DNA in each well was determined by dispensing 100 μl PICOGREEN reagent (0.25% reagent in 1×TE, v/v; Molecular Probes) and 0.5 μl of undiluted PCR product into each well of an opaque fluorimeter plate (Corning Costar, Acton Mass.) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose minigel to determine which reactions were successful in extending the sequence.

The extended nucleic acids were desalted and concentrated, transferred to 384-well plates, digested with CviJI cholera virus endonuclease (Molecular Biology Research, Madison Wis.), and sonicated or sheared prior to religation into pUC18 vector (APB). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC18 vector (APB), treated with Pfu DNA polymerase (Stratagene) to fill-in restriction site overhangs, and transfected into competent E. coli cells. Transformed cells were selected on antibiotic-containing media, and individual colonies were picked and cultured overnight at 37 C. in 384-well plates in LB/2× carbenicillin liquid media.

The cells were lysed, and DNA was amplified by PCR using Taq DNA polymerase (APB) and Pfu DNA polymerase (Stratagene) with the following parameters: Step 1: 94 C., 3 min; Step 2: 94 C., 15 sec; Step 3: 60 C., 1 min; Step 4: 72 C., 2 min; Step 5: steps 2, 3, and 4 repeated 29 times; Step 6: 72C., 5 min; Step 7: storage at 4 C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the ABI PRISM BIGDYE terminator cycle sequencing kit (ABI).

IV Assembly and Analysis of Sequences

Component nucleotide sequences from chromatograms were subjected to PHRED analysis (Phil's Revised Editing Program; Phil Green, University of Washington, Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs. Sequences were screened using the BLOCK 2 program (Incyte Genomics), a motif analysis program based on sequence information contained in the SWISS-PROT and PROSITE databases (Bairoch et al. (1997) Nucleic Acids Res 25:217-221; Attwood et al. (1997) J Chem Inf Comput Sci 37:417-424).

Processed sequences were subjected to assembly procedures in which the sequences were assigned to bins, one sequence per bin. Sequences in each bin were assembled to produce consensus sequences, templates. Subsequent new sequences were added to existing bins using the Basic Local Alignment Search Tool (BLAST; Altschul (1993) J Mol Evol 36:290-300; Altschul et al. (1990) J Mol Biol 215:403-410; Karlin et al. (1988) Proc Natl Acad Sci 85:841-845), BLASTn (v.1.4, WashU), and CROSSMATCH software (Green, supra). Candidate pairs were identified as all BLAST hits having a quality score greater than or equal to 150. Alignments of at least 82% local identity were accepted into the bin. The component sequences from each bin were assembled using PHRAP (Phil's Revised Alignment Program; Green, supra). Bins with several overlapping component sequences were assembled using DEEP PHRAP (Green, supra).

Bins were compared against each other, and those having local similarity of at least 82% were combined and reassembled. Reassembled bins having templates of insufficient overlap (less than 95% local identity) were re-split. Assembled templates were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to generate the template sequences found in the LIFESEQ GOLD database (Incyte Genomics).

The assembled templates were annotated using the following procedure. Template sequences were analyzed using BLASTn (v2.0, NCBI) versus GBpri (GenBank version 109). “Hits” were defined as an exact match having from 95% local identity over 200 base pairs through 100% local identity over 100 base pairs, or a homolog match having an E-value of 1×10⁻⁸. The hits were subjected to frameshift FASTx versus GENPEPT (GenBank version 109). In this analysis, a homolog match was defined as having an E-value of 1×10⁻⁸. The assembly method used above was described in U.S. Ser. No. 09/276,534, filed Mar. 25, 1999, incorporated by reference herein, and the LIFESEQ GOLD user manual (Incyte Genomics).

Preparation of Arrays

cDNAs were selected from The Institute for Genomic Research (TIGR) Mus.ET and NCBI GenBank mouse databases. All sequences from these sources were clustered by BLAST analysis and Smith Waterman alignment. Public domain IMAGE clones were selected to represent each of these clusters, non-redundancy and verifiable, high quality sequence were of primary concern. Therefore, the single 5′ most clone was selected as a representative of the sequence of interest, and a 5′ short read sequence was utilized to determine self-matches. Only clones that self-matched were placed on the Mouse GEM 1 array (Incyte Genomics). Sequence verified clones were mapped to clones in the public domain UniGene Mus musculus database. and the name of each clone was inherited from that process. Clones that were not clustered in the UniGene database were annotated as “IMAGE EST”. Additional annotation of the IMAGE ESTs was performed by BLAST2 analysis using blastn against other NCBI databases. To verify the sequence of each IMAGE clone, a single pass 5′ read of the sequence was produced and matched to the original sequence. Again, only clones that self-matched were placed on the array.

Purified cDNAs were immobilized on polymer-coated glass slides (Corning, Corning N.Y.) which were cleaned by ultrasound in 0.1% SDS and acetone, with extensive distilled water washes between and after treatments. The slides were etched in 4% hydrofluoric acid (VWR Scientific Products, West Chester Pa.), washed extensively in distilled water, and coated with 0.05% ainiopropyl silane (Sigma-Aldrich) in 95% ethanol. Coated slides were cured in a 110 C. oven. cDNAs were applied to the coated glass substrate using a procedure described in U.S. Pat. No. 5,807,522, incorporated herein by reference. One microliter of the cDNA at an average concentration of 100 ng/ul was loaded into the open capillary printing element by a high-speed robotic apparatus which then deposited about 5 nl of cDNA per slide.

Arrays were UV-crosslinked using a STRATALINKER UV-crosslinker (Stratagene), and then washed at room temperature once in 0.2% SDS and three times in distilled water. Non-specific binding sites were blocked by incubation of arrays in 0.2% casein in phosphate buffered saline (Tropix, Bedford Mass.) for 30 minutes at 60 C. followed by washes in 0.2% SDS and distilled water.

VI Preparation of Sample cDNAs

Mouse ES cells (E14; Hooper et al. (1987) Nature 326:292-295), derived from the inner cell mass of blastocyst stage mouse embryos, were harvested from cultures that were optimized for retention of totipotence. ES cells were cultured on a feeder layer of mitotically inactivated mouse embryonic fibroblasts in medium containing selected fetal bovine serum and β-mercaptoethanol (Hasty et al. (1991) Mol Cell Biol 11:5586-5591). Es cells were dissociated into single cells, and the fibroblasts were removed by incubating the cell suspension in a series of three tissue culture dishes for 1 to 2 hours at a time. Fibroblasts are selectively adherent to the substrate. By the third incubation period, very few adherent cells were observed. ES cells were then placed in non-adhesive petri dishes in culture medium lacking β-mercaptoethanol but containing all other components. Growth of ES cells on a nonadhesive substrate promotes the formation of multicellular aggregates known as embryoid bodies. The cells formed such aggregates after one day in culture, and the aggregates grew by accretion and proliferation over the next 4 days. The medium was changed every second day. After 4 days of culture, 5×10⁻⁷M all trans retinoic acid (RA; Sigma-Aldrich) was introduced into the culture medium and included in the medium for the next 4 days. Treatment with RA resulted in a high proportion of ES cells differentiating into neuronal-like cells (Bain et al. (1995) Dev Biol 168:342-357). After 8 days in culture, the aggregates were dissociated with trypsin and DNAse, then replated on collagen-coated dishes. Eight days later, when the cells showed extensive neurite outgrowth, the cells were harvested, and their RNA extracted.

RNA was prepared from cultures of undifferentiated cells and cultures containing ES cell-derived neurons. The mRNA was made from frozen pelleted cells by adding TRIZOL reagent (Invitrogen) and then letting the cells thaw. Total RNA was extracted using the RNA STAT-60 kit (Tel-Test, Friendswood Tex.). Poly(A) RNA was purified using the POLYATRACT mRNA isolation system (Promega). Each poly(A) RNA sample was reverse transcribed using MMLV reverse-transcriptase, 0.05 pg/μl oligo-d(T) primer (21-mer), 1× first strand buffer, 0.03 units/μl RNAse inhibitor, 500 uM dATP, 500 uM dGTP, 500 uM dTTP, 40 uM dCTP, and 40 uM either dCTP-Cy3 or dCTP-Cy5 (APB). The reverse transcription reaction was performed in a 25 ml volume containing 200 ng poly(A) RNA using the GEMBRIGHT kit (Incyte Genomics). Specific control poly(A) RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA. As quantitative controls, control mRNAs (YCFR06, YCFR45, YCFR67, and YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37 C. for 2 hr, treated with 2.5 ml of 0.5M sodium hydroxide, and incubated for 20 minutes at 85 C. to the stop the reaction and degrade the RNA. Two hundred nanograms of purified mRNA was used to produce fluorescently-labeled cDNA probes for competitive hybridization to arrays. For most experiments, probe from the parental undifferentiated cell line was labeled with Cy3, and used as the control in competitive hybridizations with Cy5-labeled probe from the experimental RA-treated cells. Probes were purified using two successive CHROMA SPIN 30 gel filtration spin columns (Clontech). Cy3- and Cy5-labeled reaction samples were combined as described below and ethanol precipitated using 1 ml of glycogen (1 mg/ml), 60 ml 5 M sodium acetate, and 300 ml of 100% ethanol. The probe was then dried to completion using a SpeedVAC system (Savant Instruments, Holbrook N.Y.) and resuspended in 14 μl 5×SSC/0.2% SDS.

VII Hybridization and Detection

Hybridization reactions contained 9 μl of probe mixture consisting of 0.2 μg each of Cy3 and Cy5 labeled cDNA synthesis products from pairs of matched time point experimental and control cells in 5×SSC, 0.2% SDS hybridization buffer. The target mixture was heated to 65 C. for 5 minutes and was aliquoted onto the surface of the Incyte Mouse GEM 1 array (Incyte Genomics) and covered with an 1.8 cm² coverslip. The arrays were transferred to a waterproof chamber having a cavity just slightly larger than a microscope slide. The chamber was kept at 100% humidity internally by the addition of 140 μl of 5×SSC in a corner of the chamber. The chamber containing the arrays was incubated for about 6.5 hours at 60 C. The arrays were washed for 10 min at 45 C. in low stringency washbuffer (1×SSC, 0.1% SDS), three times for 10 minutes each at 45 C. in high stringency wash buffer (0.1×SSC), and dried.

Reporter-labeled hybridization complexes were detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light was focused on the array using a 20× microscope objective (Nikon, Melville N.Y.). The slide containing the array was placed on a computer-controlled X-Y stage on the microscope and raster-scanned past the objective. The 1.8 cm×1.8 cm array used in the present example was scanned with a resolution of 20 micrometers.

In two separate scans, the mixed gas multiline laser excited the two fluorophores sequentially. Emitted light was split, based on wavelength, into two photomultiplier tube detectors (PMT R1477; Hamamatsu Photonics Systems, Bridgewater N.J.) corresponding to the two fluorophores. Appropriate filters positioned between the array and the photomultiplier tubes were used to filter the signals. The emission maxima of the fluorophores used were 565 nm for Cy3 and 650 nm for Cy5. Each array was typically scanned twice, one scan per fluorophore using the appropriate filters at the laser source, although the apparatus was capable of recording the spectra from both fluorophores simultaneously.

The sensitivity of the scans was calibrated using the signal intensity generated by a cDNA control species. Samples of the calibrating cDNA were separately labeled with the two fluorophores and identical amounts of each were added to the hybridization mixture. A specific location on the array contained a complementary DNA sequence, allowing the intensity of the signal at that location to be correlated with a weight ratio of hybridizing species of 1:100,000.

The output of the photomultiplier tube was digitized using a 12-bit RTI-835H analog-to-digital (A/D) conversion board (Analog Devices, Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incyte Genomics). Results from the signal analysis were presented as data in a spreadsheet (EXCEL software; Microsoft, Redmond Wash.). Data were sorted according to differential expression and tabulated.

VIII Complementary Nucleic Acid Molecules

Molecules complementary to the cDNA, or a fragment thereof, are used to detect, decrease, or inhibit gene expression. Although use of oligonucleotides comprising from about 15 to about 30 base pairs is described, the same procedure is used with larger or smaller fragments or their derivatives. Oligonucleotides are selected using OLIGO software (National Biosciences) and SEQ ID NOs: 1-278. To inhibit transcription by preventing promoter binding, a complementary oligonucleotide is designed to bind to the most unique 5′ sequence, most preferably about 10 nueleotides before the initiation codon of the open reading frame. To inhibit translation, a complementary oligonucleotide is designed to prevent ribosomal binding to the mRNA encoding the protein.

In addition to using antisense molecules constructed to interrupt transcription or translation, modifications of gene expression can be obtained by designing antisense molecules to genomic sequences (such as enhancers or introns) or even to trans-acting regulatory genes. Similarly, antisense inhibition can be achieved using Hogeboom base-pairing methodology, also known as “triple helix” base pairing. Antisense molecules involved in triple helix pairing compromise the ability of the double helix to open sufficiently for the binding of polymerases, transcription factors, or regulatory molecules.

Such antisense molecules are placed in expression vectors and used to transform preferred cells or tissues. This may include introduction of the expression vector into a cell line to test efficacy; into an organ, tumor, synovial cavity, or the vascular system for transient or short term therapy; or into a stem cell or other reproducing lineage for long term or stable gene therapy. Transient expression may last for a month or more with a non-replicating vector and for three months or more if appropriate elements for inducing vector replication are used in the transformation/expression system.

Stable transformation of appropriate dividing cells with a vector encoding the antisense molecule can produce a transgenic cell line, tissue, or organism (U.S. Pat. No. 4,736,866). Those cells that assimilate and replicate sufficient quantities of the vector to allow stable integration also produce enough antisense molecules to compromise or entirely eliminate activity of the endogenous nucleic acid.

IX Hybridization Technologies and Analyses

Hybridization technology utilizes a variety of substrates such as polymer coated glass slides and nylon membranes. Arranging elements on polymer coated slides is described in Example V; probe preparation and hybridization and analysis using polymer coated slides is described in examples VI and VII, respectively.

cDNAs are applied to a membrane substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above.

Hybridization probes derived from cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100C. for five min, and briefly centrifuging. The denatured cDNA is then added to a REDIPRIME tube (APB), gently mixed until blue color is evenly distributed, and briefly centrifuged. Five microliters of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-50 microcolumn (APB). The purified probe is heated to 100 C. for five min, snap cooled for two min on ice.

Membranes are pre-hybridized in hybridization solution containing 1% Sarkosyl and 1× high phosphate buffer (0.5 M NaCl, 0.1 M Na₂HPO₄, 5 mM EDTA, pH 7) at 55 C. for two hr. The probe, diluted in 15 ml fresh hybridization solution, is then added to the membrane. The membrane is hybridized with the probe at 55 C. for 16 hr. Following hybridization, the membrane is washed for 15 min at 25 C. in 1 mM Tris (pH 8.0), 1% Sarkosyl, and four times for 15 min each at 25 C. in 1 mM Tris (pH 8.0). To detect hybridization complexes, XOMAT-AR film (Eastman Kodak, Rochester N.Y.) is exposed to the membrane overnight at −70 C., developed, and examined.

X Transcript Image

A transcript image was produced using the LIFESEQ GOLD database (Jan02release, Incyte Genomics). This process allowed assessment of the relative abundance of the expressed polynucleotides in all of the cDNA libraries of the database. Criteria for transcript imaging can be selected from category, number of cDNAs per library, library description, disease indication, clinical relevance of sample, and the like.

All sequences and cDNA libraries in the LIFESEQ GOLD database have been categorized by system, organ/tissue and cell type. For each category, the number of libraries in which the sequence was expressed were counted and shown over the total number of libraries in that category. For each library, the number of cDNAs were counted and shown over the total number of cDNAs in that library. In some transcript images, all normalized or subtracted libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue can also be excluded where clinical relevance is emphasized. Conversely, fetal tissue can be emphasized wherever elucidation of inherited disorders or differentiation of particular adult or embryonic stem cells into tissues or organs such as heart, kidney, nerves or pancreas would be aided by removing clinical samples from the analysis.

The transcript images for SEQ ID NOs: 1376 and 2164 are shown below. The first column shows library name; the second column, the number of cDNAs sequenced in that library; the third column, the description of the library; the fourth column, absolute abundance of the transcript in the library; and the fifth column, percentage abundance of the transcript in the library.

SEQ ID NO: 1376
Category: Nervous System

| Library* | cDNAs | Description of Tissue | Abundance | % Abundance |
|---|---|---|---|---|
| HNT2NOM02 | 3158 | teratoCA line, hNT2, untreated | 7 | 0.2217 |
| HNT2NOT01 | 5782 | teratoCA line, hNT2, untreated | 7 | 0.1211 |
| HNT2TXF01 | 1144 | teratoCA line, NT2, t/cytokines | 1 | 0.0874 |
| HNT2TXC01 | 2067 | teratoCA line, hNT2, t/mouse leptin, RA | 1 | 0.0484 |
| HNT2NOM01 | 2860 | teratoCA line, hNT2, t/RA | 1 | 0.0350 |
| HNT2RAT01 | 4918 | teratoCA line, hNT2, t/RA | 1 | 0.0203 |
| HNT3AZT01 | 5219 | teratoCA line, NT2, t/5AZA-3d | 1 | 0.0192 |

*Normalized and subtracted HNT2 samples were removed from the analysis for the reasons cited above.

As can be seen above, SEQ ID NO: 1376 was differentially expressed in the untreated hNT2 libraries. It was not significantly expressed in the treated hNT2 libraries above nor in any other brain or neuronal samples in the LIFESEQ GOLD database, and it was not expressed in the treated libraries HNT2AGT01, HNT2NOM03, HNT2TXT01, and HNTNNOMO1.

SEQ ID NO: 1376
Category: Male Reproductive System

| Library* | cDNAs | Description of Tissue | Abundance | % Abundance |
|---|---|---|---|---|
| TESTTUE02 | 2642 | testis tumor, embryonal CA, 31M, 5RP | 2 | 0.0757 |
| TESTTUT02 | 7437 | testis tumor, embryonal CA, 31M, EF | 4 | 0.0538 |

*No libraries were removed from the analysis

SEQ ID NO: 1376 was not expressed in any cytologically normal or diseased prostate tissues (78 libraries), in seminoma (2 libraries) or uncharacterized tumor (1 library), or in cytologically normal (7 libraries) or necrotic testis (1 library).

In nervous system and male reproductive system, expression of SEQ ID NO: 1376 was limited to untreated teratocarcinoma cells and embryonal carcinoma of the testicles. It appears that these two kinds of germinal cancers are closely related, that embryonal cancers metastasize to the brain and elsewhere, and both the brain and testicular tumors respond to chemotherapy consisting of bleomycin, cisplatin, and vinblastine or etoposide (Yoshida and Morii (1998) J Neurosurg 88:761-3). When used in a tissue specific and clinically relevant manner, SEQ ID NO: 1376 is diagnostic of germinal tumors such as teratocarcinomas and embryonal carcinomas and can used to evaluate the efficacy of a particular treatment for this condition.

SEQ ID NO: 2164
Category: Nervous System

| Library* | cDNAs | Description of Tissue | Abundance | % Abundance |
|---|---|---|---|---|
| BRAITUT13 | 3757 | brain tumor, frontal, meningioma, 68M | 4 | 0.1065 |
| BRAITUT07 | 6240 | brain tumor, frontal, neuronal neoplasm, 32M | 2 | 0.0321 |
| BRAHDIT04 | 3190 | brain, hippocampus, AD | 1 | 0.0313 |
| BRAVTXT04 | 3922 | astrocytes, M/F, t/cytokines 4-6 hr | 1 | 0.0255 |
| BRAUTDR02 | 4706 | brain, amygdala/entorhinal cortex, 55F | 1 | 0.0212 |
| BRAENOT04 | 8909 | brain, parietal cortex, aw/CHF, 35M | 1 | 0.0112 |
| BRAITUT03 | 13222 | brain tumor, frontal, astrocytoma, 17F | 1 | 0.0076 |
| BRAITUT02 | 13385 | brain tumor, frontal, mets hypernephroma, 58M | 1 | 0.0075 |

*Libraries of fetal, normalized or subtracted, mixed or pooled tissues, and cell lines removed from analysis

SEQ ID NO: 2164 was three-fold differentially expressed in the meningioma BRAITUT13. It was not expressed in Alzheimer's disease (10 libraries), astrocytoma (7 libraries), Huntington's disease (18 libraries), multiple sclerosis (3 libraries), and schizophrenia (9 libraries). When used in a tissue specific and clinically relevant manner, SEQ ID NO: 2164 is diagnostic of meningioma and could be used to evaluate the efficacy of a particular treatment for this condition.

XI Expression of the Encoded Protein

Expression and purification of a protein encoded by a cDNA of the invention is achieved using bacterial or virus-based expression systems. For expression in bacteria, cDNA is subcloned into a vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21(DE3). Antibiotic resistant bacteria express the protein upon induction with isopropyl beta-D-thiogalactopyranoside (IPTG). Expression in eukaryotic cells is achieved by infecting Spodoptera frugiperda (Sf9) insect cells with recombinant baculovirus, Autographica californica nuclear polyhedrosis virus. The polyhedrin gene of baculovirus is replaced with the cDNA by either homologous recombination or bacterial-mediated transposition involving transfer plasmid intermediates. Viral infectivity is maintained and the strong polyhedrin promoter drives high levels of transcription.

For ease of purification, the protein is synthesized as a fusion protein with glutathione-S-transferase (GST; APB) or a similar alternative such as FLAG. The fusion protein is purified on immobilized glutathione under conditions that maintain protein activity and antigenicity. After purification, the GST moiety is proteolytically cleaved from the protein with thrombin. A fusion protein with FLAG, an 8-amino acid peptide, is purified using commercially available monoclonal and polyclonal anti-FLAG antibodies (Eastman Kodak).

XII Production of Specific Antibodies

A denatured protein from a reverse phase HPLC separation is obtained in quantities up to 75 mg. This denatured protein is used to immunize mice or rabbits following standard protocols. About 100 μg is used to immunize a mouse, while up to 1 mg is used to immunize a rabbit. The denatured protein is radioiodinated and incubated with murine B-cell hybridomas to screen for monoclonal antibodies. About 20 mg of protein is sufficient for labeling and screening several thousand clones.

In another approach, the amino acid sequence translated from a cDNA of the invention is analyzed using PROTEAN software (DNASTAR) to determine regions of high immunogenicity. The optimal sequences for immunization are usually at the C-terminus, the N-terminus, and those intervening, hydrophilic regions of the protein that are likely to be exposed to the external environment when the protein is in its natural conformation. Typically, oligopeptides about 15 residues in length are synthesized using an ABI 431 peptide synthesizer (ABI) using Fmoc-chemistry and then coupled to keyhole limpet hemocyanin (KLH; Sigma-Aldrich) by reaction with M-maleimidobenzoyl-N-hydroxysuccinimide ester. If necessary, a cysteine may be introduced at the N-terminus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated protein to identify those fusions producing a monoclonal antibody specific for the protein. In a typical protocol, wells of 96 well plates (FAST, Becton-Dickinson, Palo Alto Calif.) are coated with affinity-purified, specific rabbit-anti-mouse (or suitable anti-species Ig) antibodies at 10 mg/ml. The coated wells are blocked with 1% BSA and washed and exposed to supernatants from hybridomas. After incubation, the wells are exposed to radiolabeled protein at 1 mg/ml. Clones producing antibodies bind a quantity of labeled protein that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on protein A (APB). Monoclonal antibodies with affinities of at least 10⁸ M⁻¹, preferably 10⁹ to 10¹⁰ M⁻¹ or stronger, are made by procedures well known in the art.

XIII Purification of Naturally Occurring Protein Using Specific Antibodies

Naturally occurring or recombinant protein is substantially purified by immunoaffinity chromatography using antibodies specific for the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding

The cDNAs, proteins or antibodies of the invention are labeled with ³²P-dCTP, Cy3-dCTP, Cy5-dCTP (APB), or the proteins are labeled with BIODIPY or FITC (Molecular Probes). A library or a plurality of candidate molecules or compounds previously arranged on a substrate are incubated in the presence of labeled cDNA, protein or antibody. After incubation under conditions for a cDNA, protein, or antibody, the substrate is washed. Any position on the substrate retaining label, that indicates specific binding or complex formation, identifies a ligand. Data obtained using different concentrations of the cDNA, protein, or antibody are used to calculate affinity between the labeled molecule and the bound ligand.

All publications and patents mentioned in the specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

TABLE 1

| SEQ ID NO | RATIO | DESCRIPTION {IMAGE ID} |
|---|---|---|
| 1 | 50.49 | IGF II IMAGE EST {IMAGE: 463135} |
| 2 | 31.49 | Insulin-like growth factor binding protein 5 {IMAGE: 671661} |
| 3 | 26.26 | Insulin-like growth factor 2 {IMAGE: 464598} |
| 4 | 22.90 | KIAA0287 IMAGE EST {IMAGE: 427016} |
| 5 | 18.95 | Extracellular matrix protein 1 {IMAGE: 874833} |
| 6 | 18.58 | Procollagen, type III, alpha 1 {IMAGE: 420322} |
| 7 | 18.54 | Pleiotrophin {IMAGE: 478168} |
| 8 | 18.50 | IGF-BP 3 IMAGE EST {IMAGE: 639481} |
| 9 | 17.66 | oxoacyl reductase (acyl carrier) IMAGE EST {IMAGE: 766482} |
| 10 | 16.13 | Extracellular matrix protein 1 {IMAGE: 678765} |
| 11 | 14.56 | NEURONAL PROTEIN 3.1 {IMAGE: 425866} |
| 12 | 14.17 | Intergral membrane protein 2 {IMAGE: 532350} |
| 13 | 12.81 | NEURONAL PROTEIN 3.1 {IMAGE: 374970} |
| 14 | 12.80 | Homeo box B9 {IMAGE: 422746} |
| 15 | 11.88 | Amyloid beta (A4) precursor protein {IMAGE: 535652} |
| 16 | 10.93 | Serine protease-inhibitor 4 {IMAGE: 694987} |
| 17 | 10.84 | Serine protease-inhibitor 4 {IMAGE: 464497} |
| 18 | 10.62 | Insulin-like growth factor binding protein 5 {IMAGE: 418952} |
| 19 | 10.06 | Follistatin-like {IMAGE: 671910} |
| 20 | 9.90 | Transforming growth factor, beta 2 {IMAGE: 367780} |
| 21 | 9.78 | Procollagen, type VI, alpha 1 {IMAGE: 352450} |
| 22 | 9.34 | Procollagen, type VI, alpha 1 {IMAGE: 334132} |
| 23 | 9.24 | Insulin-like growth factor 1 {IMAGE: 313322} |
| 24 | 9.13 | fibrilin-2 IMAGE EST {IMAGE: 536526} |
| 25 | 8.75 | Procollagen, type XI, alpha 1 {IMAGE: 423028} |
| 26 | 8.66 | IMAGE EST {IMAGE: 463046} |
| 27 | 8.66 | Mouse alpha-B crystallin mRNA {IMAGE: 672201} |
| 28 | 8.65 | Carboxypeptidase E {IMAGE: 407068} |
| 29 | 8.58 | IMAGE EST {IMAGE: 482955} |
| 30 | 8.58 | Matrix metalloproteinase 2 {IMAGE: 402738} |
| 31 | 8.35 | Tissue inhibitor of metalloproteinase 2 {IMAGE: 831964} |
| 32 | 8.35 | caldesmon IMAGE EST {IMAGE: 718665} |
| 33 | 7.96 | NEURONAL PROTEIN 3.1 {IMAGE: 733420} |
| 34 | 7.86 | M. musculus mRNA for calpain-like protease {IMAGE: 747101} |
| 35 | 7.83 | frizzled-like IMAGE EST {IMAGE: 734305} |
| 36 | 7.77 | IMAGE EST {IMAGE: 468900} |
| 37 | 7.75 | Procollagen, type I, alpha 1 {IMAGE: 536306} |
| 38 | 7.70 | Ufo oncogene homolog {IMAGE: 401608} |
| 39 | 7.55 | IMAGE EST {IMAGE: 426890} |
| 40 | 7.50 | Tissue inhibitor of metalloproteinase 3 {IMAGE: 580753} |
| 41 | 7.30 | caldesmon IMAGE EST {IMAGE: 385914} |
| 42 | 7.29 | protein tyrosine kinase IMAGE EST {IMAGE: 401456} |
| 43 | 7.26 | No homolog IMAGE EST {IMAGE: 465620} |
| 44 | 7.15 | apoptosis-related BP (Napor-1) IMAGE EST {IMAGE: 620221} |
| 45 | 7.13 | Annexin V {IMAGE: 426546} |
| 46 | 7.13 | ESTs, Weakly similar to similar to alpha-actinin [C. elegans] {IMAGE: 457000} |
| 47 | 7.07 | Nuclear factor I/X {IMAGE: 367660} |
| 48 | 7.05 | IMAGE EST {IMAGE: 353202} |
| 49 | 7.05 | ESTs, Highly similar to GLYPICAN-3 PRECURSOR [Rattus norvegicus] {IMAGE: 657264} |
| 50 | 6.94 | IMAGE EST {IMAGE: 406708} |
| 51 | 6.93 | M. musculus mRNA for NfiB1-protein (exon 1-12) {IMAGE: 481927} |
| 52 | 6.91 | fibrilin-2 IMAGE EST {IMAGE: 479405} |
| 53 | 6.85 | No homolog IMAGE EST {IMAGE: 463207} |
| 54 | 6.80 | Myristoylated alanine rich protein kinase C substrate {IMAGE: 618681} |
| 55 | 6.75 | bone morphogenic protein (BMP) precursor IMAGE EST {IMAGE: 403597} |
| 56 | 6.74 | NY—CO-33(2F) IMAGE EST {IMAGE: 463342} |
| 57 | 6.67 | IMAGE EST {IMAGE: 552603} |
| 58 | 6.66 | Neural ECM/chondrocyte ezrin IMAGE EST {IMAGE: 949246} |
| 59 | 6.60 | IMAGE EST {IMAGE: 400864} |
| 60 | 6.55 | SH3-containing, STAM-associated IMAGE EST {IMAGE: 424348} |
| 61 | 6.45 | U6-snRNA-associated Sm-like protein IMAGE EST {IMAGE: 315581} |
| 62 | 6.35 | neuropilin Mus musculus IER5 (Ier5) mRNA, complete cds {IMAGE: 808000} |
| 63 | 6.33 | IMAGE EST {IMAGE: 850078} |
| 64 | 6.30 | IMAGE EST {IMAGE: 876297} |
| 65 | 6.14 | Insulin-like growth factor binding protein 10 {IMAGE: 820167} |
| 66 | 6.11 | ESTs {IMAGE: 427289} |
| 67 | 6.10 | IMAGE EST {IMAGE: 443865} |
| 68 | 6.06 | BMP-3 precursor IMAGE EST {IMAGE: 478040} |
| 69 | 5.99 | F-spondrin, neural plate protein IMAGE EST {IMAGE: 762240} |
| 70 | 5.98 | contains neural cell-type regulatory region IMAGE EST {IMAGE: 464828} |
| 71 | 5.97 | myosin regulatory light chain 2 IMAGE EST {IMAGE: 479382} |
| 72 | 5.94 | NMDA receptor Glu-binding IMAGE EST {IMAGE: 764513} |
| 73 | 5.94 | M. musculus mRNA for K-glypican {IMAGE: 314639} |
| 74 | 5.89 | endothelial actin-binding IMAGE EST {IMAGE: 315775} |
| 75 | 5.82 | melanoma-, breast-, hepatocellular CA-associated Ag ESTs, Highly similar to NECDIN [Mus musculus] |
| | | {IMAGE: 476509} |
| 76 | 5.81 | Ephrin B1 {IMAGE: 427319} |
| 77 | 5.64 | IMAGE EST {IMAGE: 427345} |
| 78 | 5.64 | Myristoylated alanine rich protein kinase C substrate {IMAGE: 617200} |
| 79 | 5.60 | IMAGE EST {IMAGE: 482995} |
| 80 | 5.58 | cystatin A-associated IMAGE EST {IMAGE: 721944} |
| 81 | 5.56 | PROTEIN-GLUTAMINE GAMMA-GLUTAMYLTRANSFERASE {IMAGE: 352804} |
| 82 | 5.54 | Mus musculus Bet1p homolog (mbet1) mRNA, complete cds {IMAGE: 333232} |
| 83 | 5.53 | ssDNA-binding ESTs {IMAGE: 634580} |
| 84 | 5.47 | nucleotide- and/or carbohydrate-binding IMAGE EST {IMAGE: 386417} |
| 85 | 5.42 | caldesmon IMAGE EST {IMAGE: 890760} |
| 86 | 5.39 | Elastin {IMAGE: 698181} |
| 87 | 5.37 | IGF II IMAGE EST {IMAGE: 422325} |
| 88 | 5.33 | transcobalamin II precursor IMAGE EST {IMAGE: 697010} |
| 89 | 5.27 | Glutathione-S-transferase, alpha 2 (Yc2) {IMAGE: 680894} |
| 90 | 5.25 | Beta-2 microglobulin {IMAGE: 572542} |
| 91 | 5.25 | Tissue inhibitor of metalloproteinase 2 {IMAGE: 902923} |
| 92 | 5.15 | IMAGE EST {IMAGE: 747941} |
| 93 | 5.14 | dopamine and/or nucleotide metabolism IMAGE EST {IMAGE: 423995} |
| 94 | 5.09 | splice variant glycogen phosphorylase isoform IMAGE EST {IMAGE: 807978} |
| 95 | 5.08 | caldesmon IMAGE EST {IMAGE: 385318} |
| 96 | 5.08 | ribosomal protein IMAGE EST {IMAGE: 352909} |
| 97 | 5.07 | No homolog IMAGE EST {IMAGE: 735413} |
| 98 | 5.05 | hu Tyr PK EPHB2 (neurogenesis-induced by RA) IMAGE EST {IMAGE: 746643} |
| 99 | 5.04 | hu profilin IMAGE EST {IMAGE: 464995} |
| 100 | 5.02 | Mus musculus protein-tyrosine phosphatase mRNA, complete cds {IMAGE: 427642} |
| 101 | 5.02 | Twist gene homolog, (Drosophila) {IMAGE: 479367} |
| 102 | 5.01 | IMAGE EST {IMAGE: 401288} |
| 103 | 4.99 | IMAGE EST {IMAGE: 332477} |
| 104 | 4.96 | profilin II IMAGE EST {IMAGE: 581193} |
| 105 | 4.95 | Homeo box A5 {IMAGE: 465937} |
| 106 | 4.92 | stromelysin-3 precursor IMAGE EST {IMAGE: 692257} |
| 107 | 4.90 | M. musculus mRNA for calpain-like protease {IMAGE: 478504} |
| 108 | 4.90 | Retinol binding protein 1, cellular {IMAGE: 406897} |
| 109 | 4.89 | KIAA0913 IMAGE EST {IMAGE: 578299} |
| 110 | 4.87 | Mus musculus protein-tyrosine phosphatase mRNA, complete cds {IMAGE: 935557} |
| 111 | 4.87 | UV B radiation-activated UV96 mRNA ESTs {IMAGE: 419146} |
| 112 | 4.83 | GATA-GT2 Zn-finger DNA binding protein IMAGE EST {IMAGE: 672424} |
| 113 | 4.81 | extracellular protein precursor IMAGE EST {IMAGE: 746798} |
| 114 | 4.80 | ESTs {IMAGE: 330825} |
| 115 | 4.79 | ESTs, Highly similar to RIBONUCLEASE PL3 PRECURSOR [Sus scrofa] {IMAGE: 803251} |
| 116 | 4.76 | Mus musculus mRNA for annexin III {IMAGE: 573265} |
| 117 | 4.72 | IMAGE EST {IMAGE: 523686} |
| 118 | 4.70 | ESTs, Highly similar to ENDOTHELIAL ACTIN-BINDING PROTEIN [Homo sapiens] {IMAGE: |
| | | 873058} |
| 119 | 4.65 | h2-calponin IMAGE EST {IMAGE: 695687} |
| 120 | 4.63 | IMAGE EST {IMAGE: 670344} |
| 121 | 4.63 | IMAGE EST {IMAGE: 481410} |
| 122 | 4.63 | keratin and LIM-domain binding protein IMAGE EST {IMAGE: 619950} |
| 123 | 4.62 | ESTs {IMAGE: 367785} |
| 124 | 4.58 | ESTs, Highly similar to HYPOTHETICAL 13.5 KD PROTEIN C45G9.7 IN CHROMOSOME III |
| | | [Caenorhabditis elegans] {IMAGE: 572510} |
| 125 | 4.58 | neuronal-associated protein IMAGE EST {IMAGE: 356215} |
| 126 | 4.55 | CD81 ANTIGEN {IMAGE: 775893} |
| 127 | 4.55 | IMAGE EST {IMAGE: 846536} |
| 128 | 4.54 | sarcosine/dimethylglycine dehydrogenase IMAGE EST {IMAGE: 420641} |
| 129 | 4.54 | LL56-APP chromosome region ESTs {IMAGE: 762558} |
| 130 | 4.54 | IMAGE EST {IMAGE: 478571} |
| 131 | 4.54 | IMAGE EST {IMAGE: 617884} |
| 132 | 4.49 | Mouse spi2 proteinase inhibitor (spi2/eb1) mRNA, 3′ end {IMAGE: 406295} |
| 133 | 4.40 | Mus musculus sodium-calcium exchanger (NCX1) mRNA, complete cds {IMAGE: 808226} |
| 134 | 4.40 | vinculin IMAGE EST {IMAGE: 765332} |
| 135 | 4.38 | ESTs, Highly similar to homologue of Drosophila Fat protein [H. sapiens] {IMAGE: 888553} |
| 136 | 4.37 | Lumican {IMAGE: 746644} |
| 137 | 4.34 | neurodegenration-associated protein 1 IMAGE EST {IMAGE: 752144} |
| 138 | 4.31 | IMAGE EST {IMAGE: 888571} |
| 139 | 4.29 | Cyclin D2 {IMAGE: 424433} |
| 140 | 4.29 | ESTs, Moderately similar to NECDIN [Mus musculus] {IMAGE: 401980} |
| 141 | 4.26 | Transforming growth factor, beta induced, 68 kDa {IMAGE: 734101} |
| 142 | 4.25 | drebrinE IMAGE EST {IMAGE: 367915} |
| 143 | 4.25 | TALIN {IMAGE: 480684} |
| 144 | 4.19 | Ste-20-like protein kinase IMAGE EST {IMAGE: 622914} |
| 145 | 4.18 | UV-B-activated transcript, polyionic rod in melanoma IMAGE EST {IMAGE: 903370} |
| 146 | 4.17 | ESTs, Highly similar to ENDOTHELIAL ACTIN-BINDING PROTEIN [Homo sapiens] {IMAGE: |
| | | 427360} |
| 147 | 4.17 | IMAGE EST {IMAGE: 402800} |
| 148 | 4.16 | IMAGE EST {IMAGE: 762159} |
| 149 | 4.16 | IMAGE EST {IMAGE: 762339} |
| 150 | 4.15 | NMDA-receptor glutamate-binding chain IMAGE EST {IMAGE: 439108} |
| 151 | 4.15 | Keratin complex 1, acidic, gene 19 {IMAGE: 464060} |
| 152 | 4.15 | IMAGE EST {IMAGE: 763103} |
| 153 | 4.14 | protein kinase C-binding protein beta IMAGE EST {IMAGE: 535763} |
| 154 | 4.13 | lipid-binding morphogenesis protein IMAGE EST {IMAGE: 820204} |
| 155 | 4.12 | ESTs {IMAGE: 373000} |
| 156 | 4.12 | IMAGE EST {IMAGE: 402995} |
| 157 | 4.08 | methyl-CpG binding protein MBD2 ESTs {IMAGE: 891284} |
| 158 | 4.08 | Murine Hox2.2 mRNA for a homeobox protein {IMAGE: 439758} |
| 159 | 4.08 | IMAGE EST {IMAGE: 425409} |
| 160 | 4.08 | TALIN {IMAGE: 334872} |
| 161 | 4.08 | Serine/Threonine protein kinase 11 IMAGE EST {IMAGE: 388477} |
| 162 | 4.08 | Myosin light chain, alkali, cardiac atria {IMAGE: 444059} |
| 163 | 4.07 | frizzled homolog IMAGE EST {IMAGE: 619970} |
| 164 | 4.06 | CD24a antigen {IMAGE: 421150} |
| 165 | 4.06 | Avian erythroblastic leukemia viral (v-erb-a) oncogene homolog-like 3 {IMAGE: 420535} |
| 166 | 4.06 | Transcription factor 12 {IMAGE: 405907} |
| 167 | 4.04 | debrin IMAGE EST {IMAGE: 467560} |
| 168 | 4.04 | CAG trinucleotide repeat protein IMAGE EST {IMAGE: 851638} |
| 169 | 4.00 | membrane glycoprotein M6 major CNS myelin protein PLP/DM20 homolog IMAGE EST {IMAGE: |
| | | 735527} |
| 170 | 3.99 | Npc1/mbac6/orphan nuclear hormone receptor/RAR-responsive/PHD-Zn-finger IMAGE EST {IMAGE: |
| | | 422403} |
| 171 | 3.94 | Proprotein convertase subtilisin/kexin type 3 {IMAGE: 680455} |
| 172 | 3.94 | Erythrocyte protein band 7.2 {IMAGE: 419756} |
| 173 | 3.93 | nucleophosmin P-protein integral nuclear envelope inner membrane protein IMAGE EST {IMAGE: |
| | | 423593} |
| 174 | 3.93 | actin-binding protein IMAGE EST {IMAGE: 408110} |
| 175 | 3.92 | Ras-like protein 2 IMAGE EST {IMAGE: 573845} |
| 176 | 3.91 | steroid oxidoreductase IMAGE EST {IMAGE: 427205} |
| 177 | 3.87 | Cadherin 2 {IMAGE: 467313} |
| 178 | 3.86 | Mouse calcineurin catalytic subunit mRNA, complete cds {IMAGE: 620546} |
| 179 | 3.85 | PDGF-beta receptor-like tumor suppressor IMAGE EST {IMAGE: 463249} |
| 180 | 3.84 | ESTs, Highly similar to CAMP-DEPENDENT PROTEIN KINASE TYPE I-ALPHA REGULATORY |
| | | CHAIN [Homo sapiens] {IMAGE: 949663} |
| 181 | 3.83 | Mus musculus MPS1 gene and mRNA, 3′ end {IMAGE: 747364} |
| 182 | 3.80 | IMAGE EST {IMAGE: 482198} |
| 183 | 3.78 | IMAGE EST {IMAGE: 317958} |
| 184 | 3.77 | Mus musculus endothelial monocyte-activating polypeptide I mRNA, complete cds {IMAGE: 669969} |
| 185 | 3.73 | Tenascin C {IMAGE: 736372} |
| 186 | 3.73 | matrix-associated protein/hbrm IMAGE EST {IMAGE: 620101} |
| 187 | 3.73 | M. musculus mRNA for selenoprotein P {IMAGE: 777018} |
| 188 | 3.72 | receptor transcriptional regulator IMAGE EST {IMAGE: 572601} |
| 189 | 3.69 | Twist gene homolog, (Drosophila) {IMAGE: 331264} |
| 190 | 3.66 | OASIS protein IMAGE EST {IMAGE: 478428} |
| 191 | 3.66 | P63 transmembrane protein ER-Golgi intermediate compartment IMAGE EST {IMAGE: 765338} |
| 192 | 3.62 | IMAGE EST {IMAGE: 657528} |
| 193 | 3.61 | ESTs {IMAGE: 425279} |
| 194 | 3.59 | IMAGE EST {IMAGE: 734718} |
| 195 | 3.59 | Mus musculus HIC-5 mRNA, complete cds {IMAGE: 426146} |
| 196 | 3.58 | Zn-finger Mok-2 IMAGE EST {IMAGE: 385763} |
| 197 | 3.57 | C1q C-chaincontains integrin receptor RGD site IMAGE EST {IMAGE: 426010} |
| 198 | 3.57 | polyhomeotic mPH2 polycomb group homolog IMAGE EST {IMAGE: 388233} |
| 199 | 3.55 | M. musculus mRNA for Ulip protein {IMAGE: 335572} |
| 200 | 3.55 | Platelet derived growth factor receptor, alpha polypeptide {IMAGE: 479895} |
| 201 | 3.55 | thyroid receptor interactor IMAGE EST {IMAGE: 385581} |
| 202 | 3.54 | Mus musculus metalloprotease/disintegrin/cysteine rich protein precursor (MDC9) cDNA, complete cds |
| | | {IMAGE: 644907} |
| 203 | 3.53 | IMAGE EST {IMAGE: 482326} |
| 204 | 3.53 | latrophilin IMAGE EST {IMAGE: 637078} |
| 205 | 3.52 | IMAGE EST {IMAGE: 425777} |
| 206 | 3.52 | Ftp-1 tyrosine phosphatase IMAGE EST {IMAGE: 400530} |
| 207 | 3.52 | GABA receptor K/Cl cotransporter IMAGE EST {IMAGE: 634792} |
| 208 | 3.52 | ESTs {IMAGE: 870973} |
| 209 | 3.49 | Mouse calcineurin catalytic subunit mRNA, complete cds {IMAGE: 699236} |
| 210 | 3.49 | hexo-, glucokinase regulator IMAGE EST {IMAGE: 466591} |
| 211 | 3.48 | TALIN {IMAGE: 676376} |
| 212 | 3.48 | ESTs {IMAGE: 621555} |
| 213 | 3.47 | TIS11B PROTEIN {IMAGE: 671377} |
| 214 | 3.47 | KIAA0444 Zn-finger helicase fucosyltransferase IMAGE EST {IMAGE: 833346} |
| 215 | 3.44 | Cyclin G {IMAGE: 849762} |
| 216 | 3.43 | adhesion/cytoskeletal IMAGE EST {IMAGE: 331794} |
| 217 | 3.42 | Annexin VI, p68 {IMAGE: 775597} |
| 218 | 3.42 | cathepsin C-like transmembrane protein IMAGE EST {IMAGE: 480854} |
| 219 | 3.40 | phospholipase C guanylate cyclase regulator IMAGE EST {IMAGE: 404044} |
| 220 | 3.39 | Mus musculus neural precursor cell expressed developmentally downregulated Nedd9 (Nedd9) mRNA, |
| | | complete cds {IMAGE: 404536} |
| 221 | 3.39 | GATA-GT2 Zn-finger IMAGE EST {IMAGE: 368189} |
| 222 | 3.38 | ATPase, Ca++ transporting, cardiac muscle, fast twitch 1 {IMAGE: 318735} |
| 223 | 3.38 | endothelial actin-binding protein IMAGE EST {IMAGE: 466292} |
| 224 | 3.36 | IMAGE EST {IMAGE: 652207} |
| 225 | 3.36 | similar to infant human clone 23587 from brain IMAGE EST {IMAGE: 807727} |
| 226 | 3.35 | ESTs, Highly similar to COMPLEMENT C1R COMPONENT PRECURSOR [Homo sapiens] |
| | | {IMAGE: 720566} |
| 227 | 3.34 | IMAGE EST {IMAGE: 871674} |
| 228 | 3.34 | IMAGE EST {IMAGE: 777549} |
| 229 | 3.32 | KIAA0992 neuronal SIH002 IMAGE EST {IMAGE: 763629} |
| 230 | 3.32 | Secreted frizzled-related sequence protein 3 {IMAGE: 803404} |
| 231 | 3.32 | semaphorin nucleolar trafficking P-protein MyD118 IMAGE EST {IMAGE: 466988} |
| 232 | 3.31 | IMAGE EST {IMAGE: 483148} |
| 233 | 3.31 | ESTs {IMAGE: 480236} |
| 234 | 3.31 | protocadherin 2C ESTs {IMAGE: 850280} |
| 235 | 3.31 | ALPHA-MANNOSIDASE II {IMAGE: 315962} |
| 236 | 3.30 | Cyclin G {IMAGE: 523713} |
| 237 | 3.30 | VITAMIN K-DEPENDENT PROTEIN S PRECURSOR {IMAGE: 681615} |
| 238 | 3.28 | Mus musculus mRNA for collagen a1(V), complete cds {IMAGE: 425344} |
| 239 | 3.27 | bone morphogenic protein-like IMAGE EST {IMAGE: 492502} |
| 240 | 3.26 | Decay accelerating factor 1 {IMAGE: 407096} |
| 241 | 3.25 | IMAGE EST {IMAGE: 634233} |
| 242 | 3.24 | IMAGE EST {IMAGE: 478021} |
| 243 | 3.24 | IMAGE EST {IMAGE: 660896} |
| 244 | 3.22 | FBJ osteosarcoma oncogene {IMAGE: 426070} |
| 245 | 3.21 | T-cell surface glycoprotein E2 precursor IMAGE EST {IMAGE: 693148} |
| 246 | 3.21 | Matrix metalloproteinase 2 {IMAGE: 762791} |
| 247 | 3.21 | IMAGE EST {IMAGE: 465043} |
| 248 | 3.21 | IMAGE EST {IMAGE: 427469} |
| 249 | 3.19 | B-cell translocation gene 2, anti-proliferative {IMAGE: 334106} |
| 250 | 3.18 | reductase IMAGE EST {IMAGE: 419544} |
| 251 | 3.18 | IMAGE EST {IMAGE: 479921} |
| 252 | 3.18 | IMAGE EST {IMAGE: 735227} |
| 253 | 3.17 | lysosomal acid lipase cholesteryl ester hydrolase precursor IMAGE EST {IMAGE: 618910} |
| 254 | 3.16 | ESTs, Weakly similar to ACTIN POLYMERIZATION INHIBITOR [Gallus gallus] {IMAGE: 426965} |
| 255 | 3.16 | Mouse spi2 proteinase inhibitor (spi2/eb1) mRNA, 3′ end {IMAGE: 640951} |
| 256 | 3.15 | ESTs {IMAGE: 677488} |
| 257 | 3.15 | Peripheral myelin protein, 22 kDa {IMAGE: 846064} |
| 258 | 3.15 | IMAGE EST {IMAGE: 330595} |
| 259 | 3.15 | Frizzled homolog 4, (Drosophila) {IMAGE: 808829} |
| 260 | 3.15 | IMAGE EST {IMAGE: 464915} |
| 261 | 3.14 | cadherin-related tumour-suppressor precursor IMAGE EST {IMAGE: 478336} |
| 262 | 3.12 | Cathepsin C {IMAGE: 876326} |
| 263 | 3.11 | Mus musculus DTEF-1 mRNA, complete cds {IMAGE: 367947} |
| 264 | 3.10 | IMAGE EST {IMAGE: 581835} |
| 265 | 3.09 | IMAGE EST {IMAGE: 333511} |
| 266 | 3.09 | voltage-gated calcium channel alpha-1-G subunit IMAGE EST {IMAGE: 475631} |
| 267 | 3.08 | ESTs, Weakly similar to TROPOMYOSIN, FIBROBLAST ISOFORM 2 [M. musculus] {IMAGE: |
| | | 643158} |
| 268 | 3.08 | HIV1-like outer envelope glycoprotein IMAGE EST {IMAGE: 749482} |
| 269 | 3.07 | IMAGE EST {IMAGE: 480454} |
| 270 | 3.07 | IMAGE EST {IMAGE: 760918} |
| 271 | 3.06 | IMAGE EST {IMAGE: 751020} |
| 272 | 3.06 | Reticulocalbin {IMAGE: 444388} |
| 273 | 3.06 | myocyte-specific enhancer-binding factor 2 IMAGE EST {IMAGE: 751385} |
| 274 | 3.05 | h2-calponin IMAGE EST {IMAGE: 402348} |
| 275 | 3.05 | nuclear targeting fetal brain HRIHFB2007 IMAGE EST {IMAGE: 420553} |
| 276 | 3.04 | IMAGE EST {IMAGE: 622257} |
| 277 | 3.04 | IMAGE EST {IMAGE: 920587} |
| 278 | 3.03 | prenylated CAAX box 1 Na—CaK regulated IMAGE EST {IMAGE: 696542} |
| 279 | 3.03 | LMP-2 IMAGE EST {IMAGE: 464575} |
| 280 | 3.03 | M. musculus mRNA for MAP kinase-activated protein kinase 2 {IMAGE: 354859} |
| 281 | 3.02 | convertase oxidoreductase IMAGE EST {IMAGE: 439383} |
| 282 | 3.01 | receptor-tyrosinase IMAGE EST {IMAGE: 764649} |
| 283 | 3.01 | IMAGE EST {IMAGE: 315890} |
| 284 | 3.01 | lipocortin p68 IMAGE EST {IMAGE: 876698} |
| 285 | 3.01 | metallocarboxypeptidase CPX-1 IMAGE EST {IMAGE: 736854} |
| 286 | 3.01 | IMAGE EST {IMAGE: 722330} |
| 287 | 3.00 | T-cell leukemia virus enhancer factor IMAGE EST {IMAGE: 444383} |
| 288 | 3.00 | tropomyosin-4 IMAGE EST {IMAGE: 890486} |
| 289 | 3.00 | kinase IMAGE EST {IMAGE: 680191} |
| 290 | 2.99 | Nidogen {IMAGE: 698175} |
| 291 | 2.98 | Mouse mRNA for plexin 2, complete cds {IMAGE: 922991} |
| 292 | 2.97 | ESTs {IMAGE: 638401} |
| 293 | 2.96 | IMAGE EST {IMAGE: 385853} |
| 294 | 2.95 | ESTs {IMAGE: 388617} |
| 295 | 2.95 | Homeo box, msh-like 2 {IMAGE: 444842} |
| 296 | 2.95 | Mouse surfeit locus surfeit 4 protein mRNA, complete cds {IMAGE: 316113} |
| 297 | 2.94 | Calcium channel, voltage-dependent, L type, alpha 2 delta subunit {IMAGE: 890932} |
| 298 | 2.93 | myeloid cell line protein IMAGE EST {IMAGE: 420591} |
| 299 | 2.93 | TOAD-64/unc-33 IMAGE EST {IMAGE: 331768} |
| 300 | 2.93 | Zn-finger P-interacting IMAGE EST {IMAGE: 864361} |
| 301 | 2.93 | ESTs, Moderately similar to lysosomal pepstatin insensitive protease [H. sapiens] {IMAGE: 574070} |
| 302 | 2.93 | BIG-1 (TAG-1/F2 Ig) IMAGE EST {IMAGE: 334291} |
| 303 | 2.92 | receptor IMAGE EST {IMAGE: 482677} |
| 304 | 2.92 | ESTs {IMAGE: 524459} |
| 305 | 2.91 | Zn-finger protein 36 IMAGE EST {IMAGE: 466781} |
| 306 | 2.91 | Lysyl oxidase {IMAGE: 418645} |
| 307 | 2.90 | IMAGE EST {IMAGE: 418766} |
| 308 | 2.90 | ESTs, Highly similar to LZIP-1 and LZIP-2 [M. musculus] {IMAGE: 680426} |
| 309 | 2.90 | ESTs, Moderately similar to lysosomal pepstatin insensitive protease [H. sapiens] {IMAGE: 776048} |
| 310 | 2.90 | neuronal cell adhesion channel with kinase/oxidase activity IMAGE EST {IMAGE: 464603} |
| 311 | 2.89 | IMAGE EST {IMAGE: 419265} |
| 312 | 2.89 | NAD/FAD-binding IMAGE EST {IMAGE: 406999} |
| 313 | 2.89 | C4/C2-activating component of Ra-reactive factor (p100) {IMAGE: 419455} |
| 314 | 2.88 | NUCLEOBINDIN PRECURSOR {IMAGE: 390016} |
| 315 | 2.88 | bup in lymphomagenesis co-regulates c-myc IMAGE EST {IMAGE: 331599} |
| 316 | 2.88 | Amino acid transporter, cationic 2 (low affinity) {IMAGE: 693244} |
| 317 | 2.87 | IMAGE EST {IMAGE: 408747} |
| 318 | 2.87 | IMAGE EST {IMAGE: 775514} |
| 319 | 2.86 | neuronal receptor fibrin-interacting IMAGE EST {IMAGE: 733937} |
| 320 | 2.86 | IMAGE EST {IMAGE: 777555} |
| 321 | 2.86 | p53-regulated PA26-T1/2/3 nuclear (nt-binding) protein IMAGE EST {IMAGE: 420049} |
| 322 | 2.85 | neuronal calcium channel/electron transfer IMAGE EST {IMAGE: 720937} |
| 323 | 2.85 | Kinesin heavy chain member 1A {IMAGE: 492514} |
| 324 | 2.85 | Mus musculus mel (MEL91) mRNA, complete cds {IMAGE: 337618} |
| 325 | 2.85 | IMAGE EST {IMAGE: 536634} |
| 326 | 2.85 | intercellular signal transducer/transmitter Fz-1 IMAGE EST {IMAGE: 459284} |
| 327 | 2.84 | myosin-interacting IMAGE EST {IMAGE: 408381} |
| 328 | 2.84 | contains E. coli sequences IMAGE EST {IMAGE: 420484} |
| 329 | 2.83 | ESTs, Moderately similar to bone-derived growth factor [H. sapiens] {IMAGE: 458802} |
| 330 | 2.83 | IMAGE EST {IMAGE: 425742} |
| 331 | 2.82 | death (apoptosis)-associated protein kinase-1 IMAGE EST {IMAGE: 932999} |
| 332 | 2.82 | ESTs, Weakly similar to C01A2.5 [C. elegans] {IMAGE: 735647} |
| 333 | 2.81 | Sec24 protein IMAGE EST {IMAGE: 634252} |
| 334 | 2.81 | ESTs {IMAGE: 550700} |
| 335 | 2.81 | galactoprotein b3 IMAGE EST {IMAGE: 597249} |
| 336 | 2.81 | IMAGE EST {IMAGE: 620453} |
| 337 | 2.81 | IMAGE EST {IMAGE: 464803} |
| 338 | 2.81 | procollagen alpha2 chain IMAGE EST {IMAGE: 445075} |
| 339 | 2.80 | collagen XIV IMAGE EST {IMAGE: 472722} |
| 340 | 2.79 | ESTs {IMAGE: 738162} |
| 341 | 2.79 | ESTs {IMAGE: 642114} |
| 342 | 2.79 | Wingless-related MMTV integration site 3A {IMAGE: 426103} |
| 343 | 2.79 | IMAGE EST {IMAGE: 764212} |
| 344 | 2.78 | KIAA0382 KIAA0380 TIAM-1-like protein IMAGE EST {IMAGE: 523303} |
| 345 | 2.78 | KIAA0671 IMAGE EST {IMAGE: 736017} |
| 346 | 2.77 | IMAGE EST {IMAGE: 803488} |
| 347 | 2.77 | Mus musculus FK506-binding protein (FKBP23) mRNA, complete cds {IMAGE: 656375} |
| 348 | 2.77 | kinase growth modulator IMAGE EST {IMAGE: 920701} |
| 349 | 2.76 | IMAGE EST {IMAGE: 492457} |
| 350 | 2.76 | glycogen synthase kinase-3 beta IMAGE EST {IMAGE: 736232} |
| 351 | 2.76 | Cell adhesion kinase {IMAGE: 424307} |
| 352 | 2.76 | B-cell translocation gene 2, anti-proliferative {IMAGE: 583186} |
| 353 | 2.75 | homeobox-containing similar to HoxA8, -B8, -D8 IMAGE EST {IMAGE: 418660} |
| 354 | 2.74 | IMAGE EST {IMAGE: 493238} |
| 355 | 2.74 | Amyloid beta (A4) precursor-like protein 2 {IMAGE: 876058} |
| 356 | 2.74 | Mus musculus fibrillin 2 (fbn2) gene, complete cds {IMAGE: 617885} |
| 357 | 2.74 | ESTs {IMAGE: 419185} |
| 358 | 2.73 | IMAGE EST {IMAGE: 736526} |
| 359 | 2.72 | Mus musculus liver carnitine palmitoyltransferase I mRNA, partial cds {IMAGE: 737898} |
| 360 | 2.72 | Mus musculus mRNA for membrane type-2 matrix metalloproteinase, complete cds {IMAGE: 680964} |
| 361 | 2.72 | beta-galactosidase alpha 2,6-sialyltransferase IMAGE EST {IMAGE: 481883} |
| 362 | 2.71 | receptor IMAGE EST {IMAGE: 423599} |
| 363 | 2.70 | IMAGE EST {IMAGE: 636407} |
| 364 | 2.70 | AHPC/TSA protein thio-specific antioxidant peroxidase IMAGE EST {IMAGE: 481450} |
| 365 | 2.70 | PITP kinase IMAGE EST {IMAGE: 599075} |
| 366 | 2.70 | ESTs {IMAGE: 573052} |
| 367 | 2.70 | ESTs {IMAGE: 464165} |
| 368 | 2.70 | ESTs {IMAGE: 622039} |
| 369 | 2.70 | Mouse procollagen type V alpha 2 (Col5a-2) mRNA, complete cds {IMAGE: 467107} |
| 370 | 2.69 | MBLL C3H Zn-finger protein IMAGE EST {IMAGE: 807447} |
| 371 | 2.69 | calcium-transporting ATPase plasma membrane IMAGE EST {IMAGE: 716523} |
| 372 | 2.69 | kinase IMAGE EST {IMAGE: 733746} |
| 373 | 2.69 | IMAGE EST {IMAGE: 817954} |
| 374 | 2.68 | phosphatase PP2A 55 kDa regulatory subunit IMAGE EST {IMAGE: 466730} |
| 375 | 2.68 | IMAGE EST {IMAGE: 763553} |
| 376 | 2.68 | PEPTIDYL-PROLYL CIS-TRANS ISOMERASE C {IMAGE: 406947} |
| 377 | 2.68 | cytoskeletal-associated lipid-interacting protein IMAGE EST {IMAGE: 622182} |
| 378 | 2.68 | ephrin receptor/hormone signal mediator; splicing factor IMAGE EST {IMAGE: 422477} |
| 379 | 2.67 | IMAGE EST {IMAGE: 949384} |
| 380 | 2.67 | IMAGE EST {IMAGE: 777321} |
| 381 | 2.67 | extracellular matrix-interacting, guanylate cyclase/ANF-like protein IMAGE EST {IMAGE: 475285} |
| 382 | 2.66 | IMAGE EST {IMAGE: 493702} |
| 383 | 2.65 | human fetal brain RIG-like 7-1 protein IMAGE EST {IMAGE: 536577} |
| 384 | 2.65 | receptor-kinase protein IMAGE EST {IMAGE: 466255} |
| 385 | 2.65 | N-copine I and III IMAGE EST {IMAGE: 466178} |
| 386 | 2.65 | IMAGE EST {IMAGE: 464415} |
| 387 | 2.65 | receptor tumour suppressor IMAGE EST {IMAGE: 423344} |
| 388 | 2.63 | KIAA0964 IMAGE EST {IMAGE: 637333} |
| 389 | 2.63 | Myosin heavy chain, skeletal muscle, embryonic {IMAGE: 439638} |
| 390 | 2.63 | Cut (Drosphila)-like 1 {IMAGE: 458712} |
| 391 | 2.63 | myeloid associated differentiation protein ESTs {IMAGE: 672859} |
| 392 | 2.62 | Parathyroid hormone receptor {IMAGE: 467172} |
| 393 | 2.61 | IMAGE EST {IMAGE: 400571} |
| 394 | 2.61 | IMAGE EST {IMAGE: 355517} |
| 395 | 2.61 | electron transfer IMAGE EST {IMAGE: 777529} |
| 396 | 2.61 | pyruvate dehydrogenase kinase 2 p45 subunit IMAGE EST {IMAGE: 533405} |
| 397 | 2.61 | IMAGE EST {IMAGE: 680761} |
| 398 | 2.60 | IMAGE EST {IMAGE: 374296} |
| 399 | 2.60 | tensin IMAGE EST {IMAGE: 774810} |
| 400 | 2.60 | transmembrane protein adrenal-associated IMAGE EST {IMAGE: 582063} |
| 401 | 2.59 | ESTs {IMAGE: 643905} |
| 402 | 2.59 | IMAGE EST {IMAGE: 484125} |
| 403 | 2.58 | DNA repair IMAGE EST {IMAGE: 332327} |
| 404 | 2.58 | Guanine nucleotide binding protein, alpha stimulating {IMAGE: 717466} |
| 405 | 2.58 | methyl-CpG binding protein MBD2 ESTs {IMAGE: 421592} |
| 406 | 2.57 | Max-interacting transcriptional represser IMAGE EST {IMAGE: 583632} |
| 407 | 2.57 | Glycoprotein galactosyltransferase alpha 1, 3 {IMAGE: 618535} |
| 408 | 2.57 | phosphatidylinositol-3-kinase regulator IMAGE EST {IMAGE: 761605} |
| 409 | 2.56 | AP-1 regulated gene product IMAGE EST {IMAGE: 693542} |
| 410 | 2.56 | Prostaglandin E receptor EP1 subtype {IMAGE: 751958} |
| 411 | 2.56 | c-Maf IMAGE EST {IMAGE: 749073} |
| 412 | 2.56 | Mannosidase 2, alpha B1 {IMAGE: 484259} |
| 413 | 2.56 | Fus-2 tumour suppressor IMAGE EST {IMAGE: 479920} |
| 414 | 2.55 | ESTs {IMAGE: 891260} |
| 415 | 2.55 | ESTs {IMAGE: 478904} |
| 416 | 2.54 | thymus-associated proteins IMAGE EST {IMAGE: 597342} |
| 417 | 2.54 | preprotakykinin peptide receptor IMAGE EST {IMAGE: 439399} |
| 418 | 2.54 | IMAGE EST {IMAGE: 638302} |
| 419 | 2.53 | Lamin A {IMAGE: 524119} |
| 420 | 2.53 | glutamat aspartate transporter IMAGE EST {IMAGE: 864344} |
| 421 | 2.53 | channel/receptor IMAGE EST {IMAGE: 695508} |
| 422 | 2.53 | talin IMAGE EST {IMAGE: 467480} |
| 423 | 2.52 | ESTs, Highly similar to TRANSLOCON-ASSOCIATED PROTEIN, GAMMA SUBUNIT [Rattus |
| | | norvegicus] {IMAGE: 404526} |
| 424 | 2.52 | microtubule vesicle linker clip protein IMAGE EST {IMAGE: 439732} |
| 425 | 2.51 | growth factor signalling antagonist IMAGE EST {IMAGE: 403274} |
| 426 | 2.51 | CaM-regulated CoA-utilizing enzyme IMAGE EST {IMAGE: 426493} |
| 427 | 2.51 | IMAGE EST {IMAGE: 576974} |
| 428 | 2.50 | cytoskeleton-interacting protein IMAGE EST {IMAGE: 720567} |
| 429 | 2.50 | trithorax homolog/Rb-related p130 homolog IMAGE EST {IMAGE: 478156} |
| 430 | 2.49 | G-protein gamma7 subunit IMAGE EST {IMAGE: 641871} |
| 431 | 2.49 | Protein tyrosine phosphatase, receptor type, A {IMAGE: 622319} |
| 432 | 2.49 | zinc finger MBNL protein ESTs {IMAGE: 717910} |
| 433 | 2.49 | nuclear proto-oncogene protease inhibitor lipid-binding IMAGE EST {IMAGE: 476653} |
| 434 | 2.48 | ESTs {IMAGE: 387151} |
| 435 | 2.48 | tumour suppressor IMAGE EST {IMAGE: 762532} |
| 436 | 2.48 | ESTs {IMAGE: 762728} |
| 437 | 2.48 | c-fos IMAGE EST {IMAGE: 439803} |
| 438 | 2.48 | ESTs {IMAGE: 874540} |
| 439 | 2.47 | GP25L2 protein IMAGE EST {IMAGE: 831744} |
| 440 | 2.47 | IMAGE EST {IMAGE: 764518} |
| 441 | 2.47 | Mus musculus brain fatty acid-binding protein (B-FABP) gene, complete cds {IMAGE: 436894} |
| 442 | 2.47 | IMAGE EST {IMAGE: 473770} |
| 443 | 2.46 | IMAGE EST {IMAGE: 438506} |
| 444 | 2.46 | EF-hand calcium-binding p22 IMAGE EST {IMAGE: 580505} |
| 445 | 2.46 | P24A membrane protein IMAGE EST {IMAGE: 582571} |
| 446 | 2.46 | Erythrocyte protein band 7.2 {IMAGE: 481880} |
| 447 | 2.46 | ESTs, Highly similar to GLYCINE AMIDINOTRANSFERASE PRECURSOR [R. norvegicus] {IMAGE: |
| | | 478521} |
| 448 | 2.46 | KIAA0470 IMAGE EST {IMAGE: 421537} |
| 449 | 2.45 | transmembrane receptor IMAGE EST { IMAGE: 809031} |
| 450 | 2.45 | Sel1l-negative regulator of notch IMAGE EST {IMAGE: 390236} |
| 451 | 2.44 | NDP transporter/receptor IMAGE EST {IMAGE: 733517} |
| 452 | 2.44 | ESTs {IMAGE: 597547} |
| 453 | 2.44 | laminin-beta IMAGE EST {IMAGE: 418495} |
| 454 | 2.43 | Mus musculus caveolin-1 mRNA, complete cds {IMAGE: 331186} |
| 455 | 2.43 | Mus musculus protein co-factor mRNA, complete cds {IMAGE: 694133} |
| 456 | 2.43 | ESTs, Highly similar to hypertension-related protein [R. norvegicus] {IMAGE: 776562} |
| 457 | 2.43 | Mus musculus WSB-1 mRNA, complete cds {IMAGE: 619201} |
| 458 | 2.42 | GalBeatGalNAcAlpha sialyltransferase IMAGE EST {IMAGE: 418453} |
| 459 | 2.42 | Mok-2 Zn-finger protein IMAGE EST {IMAGE: 440843} |
| 460 | 2.41 | Mus musculus LIM protein FHL2 (Fhl2) mRNA, complete cds {IMAGE: 457264} |
| 461 | 2.41 | IMAGE EST {IMAGE: 332687} |
| 462 | 2.41 | ESTs {IMAGE: 598305} |
| 463 | 2.41 | synaptomagnin, protein kinase C, and guanyl-binding protein IMAGE EST {IMAGE: 466905} |
| 464 | 2.40 | arginine methyltransferase hnRNP IMAGE EST {IMAGE: 574888} |
| 465 | 2.40 | ESTs, Moderately similar to Similar to a C. elegans protein in cosmid C14H10 [H. sapiens] {IMAGE: |
| | | 679641} |
| 466 | 2.40 | IMAGE EST {IMAGE: 734062} |
| 467 | 2.40 | IMAGE EST {IMAGE: 576216} |
| 468 | 2.40 | MAD homolog 1 (Drosophila) {IMAGE: 620333} |
| 469 | 2.39 | protease IMAGE EST {IMAGE: 441276} |
| 470 | 2.39 | ApoA4-like, -regulating protein IMAGE EST {IMAGE: 476019} |
| 471 | 2.39 | ESTs, Highly similar to ER LUMEN PROTEIN RETAINING RECEPTOR 2 [Homo sapiens] {IMAGE: |
| | | 354601} |
| 472 | 2.39 | tropomyosin-like IMAGE EST {IMAGE: 474107} |
| 473 | 2.39 | desmoplakin I and I IMAGE EST {IMAGE: 420709} I |
| 474 | 2.38 | Mus musculus mRNA for Rab33B, complete cds {IMAGE: 621389} |
| 475 | 2.38 | cell surface protein, yeast YKKO-like IMAGE EST {IMAGE: 717316} |
| 476 | 2.38 | WD40 repeat protein IMAGE EST {IMAGE: 493109} |
| 477 | 2.37 | Aryl-hydrocarbon receptor {IMAGE: 403384} |
| 478 | 2.37 | IMAGE EST {IMAGE: 483024} |
| 479 | 2.37 | BS69 IMAGE EST {IMAGE: 572889} |
| 480 | 2.37 | ESTs, Moderately similar to developmentally regulated protein [R. norvegicus] {IMAGE: 733557} |
| 481 | 2.37 | Protein kinase, cAMP dependent, catalytic, beta {IMAGE: 478265} |
| 482 | 2.36 | src SH3-binding ADP-ribosylation factor-directed GTPase activating protein IMAGE EST {IMAGE: |
| | | 423915} |
| 483 | 2.36 | IMAGE EST {IMAGE: 695447} |
| 484 | 2.36 | IMAGE EST {IMAGE: 640875} |
| 485 | 2.36 | N-acetylglucosamine-sulphatase precursor IMAGE EST {IMAGE: 480526} |
| 486 | 2.36 | rearranged lipid-interacting cell surface protein IMAGE EST {IMAGE: 696489} |
| 487 | 2.36 | ESTs {IMAGE: 639911} |
| 488 | 2.36 | KIAA0581 phospholipase C-beta1b IMAGE EST {IMAGE: 874154} |
| 489 | 2.36 | Complement component factor h {IMAGE: 777640} |
| 490 | 2.36 | IMAGE EST {IMAGE: 888624} |
| 491 | 2.35 | IMAGE EST {IMAGE: 762542} |
| 492 | 2.35 | ESTs, Weakly similar to VILLIN [Gallus gallus] {IMAGE: 875543} |
| 493 | 2.35 | IMAGE EST {IMAGE: 439959} |
| 494 | 2.35 | IMAGE EST {IMAGE: 749144} |
| 495 | 2.35 | ESTs, Weakly similar to RING zinc finger protein [M. musculus] {IMAGE: 922965} |
| 496 | 2.33 | ESTs {IMAGE: 721040} |
| 497 | 2.33 | IMAGE EST {IMAGE: 480149} |
| 498 | 2.33 | prothymosin-like IMAGE EST {IMAGE: 596754} |
| 499 | 2.33 | cell adhesion regulator-1 IMAGE EST {IMAGE: 920211} |
| 500 | 2.32 | IMAGE EST {IMAGE: 457289} |
| 501 | 2.32 | Chondroitin sulfate proteoglycan 2 {IMAGE: 355990} |
| 502 | 2.32 | dual-specific nuclear matrix tyrosine protein kinase IMAGE EST {IMAGE: 354506} |
| 503 | 2.31 | regulator of TGF-beta-induced activity IMAGE EST {IMAGE: 424979} |
| 504 | 2.31 | ESTs {IMAGE: 697506} |
| 505 | 2.31 | IMAGE EST {IMAGE: 419788} |
| 506 | 2.30 | Cystatin 3 {IMAGE: 402614} |
| 507 | 2.30 | serine/threonine protein kinase IMAGE EST {IMAGE: 459254} |
| 508 | 2.30 | myeloid associated differentiation protein ESTs {IMAGE: 672351} |
| 509 | 2.29 | PSD-95/SAP90-associated protein-4 IMAGE EST {IMAGE: 466715} |
| 510 | 2.29 | ESTs, Weakly similar to QUINONE OXIDOREDUCTASE [M. musculus] {IMAGE: 735186} |
| 511 | 2.29 | lipase/synthetase IMAGE EST {IMAGE: 314394} |
| 512 | 2.28 | IMAGE EST {IMAGE: 748241} |
| 513 | 2.28 | IMAGE EST {IMAGE: 421973} |
| 514 | 2.28 | ESTs {IMAGE: 621316} |
| 515 | 2.28 | ESTs {IMAGE: 404339} |
| 516 | 2.28 | Mus musculus krupple-related zinc finger protein (Emzfl) mRNA, complete cds {IMAGE: 764361} |
| 517 | 2.28 | metal-binding protein IMAGE EST {IMAGE: 482328} |
| 518 | 2.28 | ESTs, Moderately similar to HYPOTHETICAL 13.6 KD PROTEIN IN NUP170-ILS1 INTERGENIC |
| | | REGION [S. cerevisiae] {IMAGE: 775912} |
| 519 | 2.28 | ESTs {IMAGE: 571643} |
| 520 | 2.27 | Thymoma viral proto-oncogene {IMAGE: 678740} |
| 521 | 2.27 | ESTs {IMAGE: 618644} |
| 522 | 2.27 | Receptor interacting protein {IMAGE: 763021} |
| 523 | 2.27 | ESTs, Weakly similar to coded for by C. elegans cDNA yk86e5.5 [C. elegans] {IMAGE: 641451} |
| 524 | 2.26 | alcohol dehydrogenase IMAGE EST {IMAGE: 314127} |
| 525 | 2.26 | Mus musculus caveolin-1 mRNA, complete cds {IMAGE: 596968} |
| 526 | 2.26 | Mus musculus parathion hydrolase (phosphotriesterase)-related protein mRNA, complete cds {IMAGE: |
| | | 803358} |
| 527 | 2.26 | ESTs, Highly similar to T-KININOGEN II PRECURSOR [Rattus norvegicus] {IMAGE: 390390} |
| 528 | 2.26 | Mus musculus G protein-coupled receptor kinase 5 (GRK5) mRNA, complete cds {IMAGE: 875444} |
| 529 | 2.25 | slit protein precursor IMAGE EST {IMAGE: 820354} |
| 530 | 2.25 | Laminin, alpha 4 {IMAGE: 736616} |
| 531 | 2.24 | integrin beta/elastin IMAGE EST {IMAGE: 876166} |
| 532 | 2.24 | ESTs, Weakly similar to schwannoma-associated protein [M. musculus] {IMAGE: 904738} |
| 533 | 2.23 | Mus musculus mRNA for mtprd, complete cds {IMAGE: 775150} |
| 534 | 2.23 | ESTs, Highly similar to VACUOLAR ATP SYNTHASE SUBUNIT AC45 PRECURSOR [Bos taurus] |
| | | {IMAGE: 671660} |
| 535 | 2.23 | CALPONIN H1, SMOOTH MUSCLE {IMAGE: 373793} |
| 536 | 2.22 | ESTs {IMAGE: 596200} |
| 537 | 2.22 | IMAGE EST {IMAGE: 652498} |
| 538 | 2.22 | IMAGE EST {IMAGE: 658233} |
| 539 | 2.22 | ESTs {IMAGE: 442842} |
| 540 | 2.22 | IMAGE EST {IMAGE: 774877} |
| 541 | 2.22 | ESTs, Weakly similar to PROBABLE PROTEIN DISULFIDE ISOMERASE ER-60 PRECURSOR |
| | | [M. musculus] {IMAGE: 421530} |
| 542 | 2.22 | filament forming steroid-interacting protein IMAGE EST {IMAGE: 419553} |
| 543 | 2.21 | IMAGE EST {IMAGE: 386236} |
| 544 | 2.21 | excised intron-binding RNA IMAGE EST {IMAGE: 418531} |
| 545 | 2.21 | smoothened seven transmembrane receptor growth regulator IMAGE EST {IMAGE: 439010} |
| 546 | 2.21 | IMAGE EST {IMAGE: 643384} |
| 547 | 2.21 | IMAGE EST {IMAGE: 464245} |
| 548 | 2.20 | IMAGE EST {IMAGE: 718110} |
| 549 | 2.20 | similar to H. sapiens chromosome 17 ESTs {IMAGE: 902264} |
| 550 | 2.20 | Erythropoietin receptor {IMAGE: 718293} |
| 551 | 2.19 | Calumenin {IMAGE: 523088} |
| 552 | 2.19 | phosphoeicosinoid isomerase IMAGE EST {IMAGE: 423219} |
| 553 | 2.19 | ESTs {IMAGE: 762782} |
| 554 | 2.19 | Mus musculus tetraspanin Tspan-6 (Tspan-6) mRNA, complete cds {IMAGE: 572156} |
| 555 | 2.19 | cerebroglycan IMAGE EST {IMAGE: 763989} |
| 556 | 2.18 | RNA processing IMAGE EST {IMAGE: 463860} |
| 557 | 2.18 | C1 channel protein P64 IMAGE EST {IMAGE: 670375} |
| 558 | 2.18 | Mus musculus metalloprotease-disintegrin MDC15 mRNA, complete cds {IMAGE: 406689} |
| 559 | 2.18 | IMAGE EST {IMAGE: 466155} |
| 560 | 2.18 | ESTs {IMAGE: 622792} |
| 561 | 2.18 | T-cell surface protein tactile precursor IMAGE EST {IMAGE: 597729} |
| 562 | 2.17 | ESTs {IMAGE: 472407} |
| 563 | 2.17 | Zn-finger TATA-box-binding protein associated factor (TAF) IMAGE EST {IMAGE: 765073} |
| 564 | 2.16 | protocadherin IMAGE EST {IMAGE: 922216} |
| 565 | 2.16 | Mus musculus Nip21 mRNA, complete cds {IMAGE: 677412} |
| 566 | 2.16 | IMAGE EST {IMAGE: 722451} |
| 567 | 2.16 | IMAGE EST {IMAGE: 846131} |
| 568 | 2.16 | Zn-finger DNA-binding protein IMAGE EST {IMAGE: 762142} |
| 569 | 2.16 | ESTs {IMAGE: 934635} |
| 570 | 2.15 | Mus musculus sphingosine-1-phosphate lyase mRNA, complete cds {IMAGE: 332091} |
| 571 | 2.15 | Cadherin 3 {IMAGE: 329780} |
| 572 | 2.15 | ESTs {IMAGE: 574476} |
| 573 | 2.15 | IMAGE EST {IMAGE: 476342} |
| 574 | 2.15 | spindlin G-protein effector chaperone IMAGE EST {IMAGE: 482943} |
| 575 | 2.15 | IMAGE EST {IMAGE: 680103} |
| 576 | 2.14 | IMAGE EST {IMAGE: 574134} |
| 577 | 2.14 | IMAGE EST {IMAGE: 334410} |
| 578 | 2.14 | ESTs, Highly similar to S-100 PROTEIN, ALPHA CHAIN [Rattus norvegicus] {IMAGE: 872869} |
| 579 | 2.13 | KIAA0623 Unc-51-like kinase 2 IMAGE EST {IMAGE: 749776} |
| 580 | 2.13 | ESTs, Moderately similar to Similar to a C. elegans protein in cosmid C14H10 [H. sapiens] {IMAGE: |
| | | 749282} |
| 581 | 2.13 | Glycoprotein 49 B {IMAGE: 820498} |
| 582 | 2.13 | IMAGE EST {IMAGE: 317778} |
| 583 | 2.13 | Catenin Src {IMAGE: 439784} |
| 584 | 2.13 | retinal protein IMAGE EST {IMAGE: 426076} |
| 585 | 2.12 | KIAA0613 enigma/LIM2 IMAGE EST {IMAGE: 336726} |
| 586 | 2.12 | ESTs {IMAGE: 479750} |
| 587 | 2.12 | IMAGE EST {IMAGE: 353229} |
| 588 | 2.12 | MADS/MEF2family transcription factor IMAGE EST {IMAGE: 777101} |
| 589 | 2.11 | myosin light chain, slow-twitch isoform IMAGE EST {IMAGE: 330370} |
| 590 | 2.11 | NG, NG-dimethylargine dimethylaminohydrolase IMAGE EST {IMAGE: 656701} |
| 591 | 2.11 | IMAGE EST {IMAGE: 622653} |
| 592 | 2.11 | kinetocore growth-associated containing B1-repeat (rodent) IMAGE EST {IMAGE: 425348} |
| 593 | 2.11 | Gt12 myogenic and CNS development GPCR IMAGE EST {IMAGE: 423177} |
| 594 | 2.11 | endothelin-converting enzyme-1 IMAGE EST {IMAGE: 419633} |
| 595 | 2.10 | PAK2, caspase 3 substrate, apoptosis-related ESTs {IMAGE: 635569} |
| 596 | 2.10 | ESTs {IMAGE: 493460} |
| 597 | 2.10 | Ig lambda chain C region IMAGE EST {IMAGE: 777306} |
| 598 | 2.10 | G-protein interacting receptor IMAGE EST {IMAGE: 761298} |
| 599 | 2.10 | cytoskeletal protein IMAGE EST {IMAGE: 479846} |
| 600 | 2.10 | IMAGE EST {IMAGE: 595978} |
| 601 | 2.10 | IMAGE EST {IMAGE: 850389} |
| 602 | 2.10 | Similar to tip of human chromosome 16 ESTs {IMAGE: 888687} |
| 603 | 2.10 | Neurotrophic tyrosine kinase, receptor, type 3 {IMAGE: 478848} |
| 604 | 2.10 | KIAA0662 RAR-inducible factor growth arrest IMAGE EST {IMAGE: 477665} |
| 605 | 2.09 | IMAGE EST {IMAGE: 421653} |
| 606 | 2.09 | Krupple-associated box (KRAB)-containing Zn-finger IMAGE EST {IMAGE: 622359} |
| 607 | 2.09 | membrane protein IMAGE EST {IMAGE: 637536} |
| 608 | 2.09 | Mast cell protease 5 {IMAGE: 425653} |
| 609 | 2.09 | IMAGE EST {IMAGE: 641660} |
| 610 | 2.09 | Mus musculus Ena-VASP like protein (Ev1) mRNA, complete cds {IMAGE: 477177} |
| 611 | 2.08 | similar to heart-derived DNA ESTs {IMAGE: 419846} |
| 612 | 2.08 | Fyn protooncogene {IMAGE: 385072} |
| 613 | 2.08 | IMAGE EST {IMAGE: 935626} |
| 614 | 2.08 | Thrombospondin 3 {IMAGE: 483763} |
| 615 | 2.07 | semaphorin III cdc-like NAD/FAD-binding protein IMAGE EST {IMAGE: 476321} |
| 616 | 2.07 | Mus musculus uncoupling protein homolog (UCPH) mRNA, complete cds {IMAGE: 748112} |
| 617 | 2.07 | Zinc finger protein 36 {IMAGE: 641523} |
| 618 | 2.07 | IMAGE EST {IMAGE: 422094} |
| 619 | 2.07 | Mus musculus palmitoyl-protein thioesterase precursor, mRNA, complete cds {IMAGE: 637934} |
| 620 | 2.07 | Mus musculus syntaxin 7 (Syn7) mRNA, complete cds {IMAGE: 468472} |
| 621 | 2.07 | homeobox-containing Janus kinase (Jak3) tyrosine kinase IMAGE EST {IMAGE: 641806} |
| 622 | 2.06 | Notch2-like EGF-repeat transmembrane protein IMAGE EST {IMAGE: 621352} |
| 623 | 2.06 | CoA: amino acid N-acyltransferase IMAGE EST {IMAGE: 775722} |
| 624 | 2.06 | voltage-gated calcium channel IMAGE EST {IMAGE: 638593} |
| 625 | 2.06 | KIAA0198 myoblast Zn-finger protein IMAGE EST {IMAGE: 406222} |
| 626 | 2.06 | Trg gene product IMAGE EST {IMAGE: 583823} |
| 627 | 2.06 | hypothalamic estrogen-induced geme in female adult rat IMAGE EST {IMAGE: 571503} |
| 628 | 2.06 | Mus musculus S182 protein mRNA, complete cds {IMAGE: 681670} |
| 629 | 2.06 | ADP-ribosylation factor 2 ESTs {IMAGE: 721482} |
| 630 | 2.06 | Mus musculus mRNA for NBAT, complete cds {IMAGE: 776770} |
| 631 | 2.05 | neuronal glucose transporter IMAGE EST {IMAGE: 423210} |
| 632 | 2.05 | glycophorin C IMAGE EST {IMAGE: 598956} |
| 633 | 2.04 | CaM-1 IMAGE EST {IMAGE: 660997} |
| 634 | 2.04 | KIAA0317 IMAGE EST {IMAGE: 751729} |
| 635 | 2.04 | receptor tyrosine phosphatase IMAGE EST {IMAGE: 535228} |
| 636 | 2.04 | IMAGE EST {IMAGE: 464134} |
| 637 | 2.04 | bone morphogenic protein 5 precursor/Vgr-1 protein IMAGE EST {IMAGE: 737934} |
| 638 | 2.04 | ESTs, Highly similar to unknown [M. Musculus] {IMAGE: 697509} |
| 639 | 2.04 | erythrocyte protein 4.2 IMAGE EST {IMAGE: 481375} |
| 640 | 2.03 | Calcium channel beta 3 subunit {IMAGE: 420487} |
| 641 | 2.03 | RP58 Zn-finger POZ-domain IMAGE EST {IMAGE: 574756} |
| 642 | 2.03 | Janus kinase 2 {IMAGE: 621226} |
| 643 | 2.03 | IMAGE EST {IMAGE: 480431} |
| 644 | 2.03 | Zn-finger protein 22 IMAGE EST {IMAGE: 427349} |
| 645 | 2.03 | beta-TrCP ESTs {IMAGE: 699290} |
| 646 | 2.03 | M. musculus rhoC mRNA {IMAGE: 670886} |
| 647 | 2.03 | thyroid receptor interactor IMAGE EST {IMAGE: 385725} |
| 648 | 2.03 | IMAGE EST {IMAGE: 818435} |
| 649 | 2.03 | Mus musculus liver carnitine palmitoyltransferase I mRNA, partial cds {IMAGE: 717056} |
| 650 | 2.03 | KIAA0630 IMAGE EST {IMAGE: 385435} |
| 651 | 2.02 | Fos-related antigen2 IMAGE EST {IMAGE: 445817} |
| 652 | 2.02 | IMAGE EST {IMAGE: 949003} |
| 653 | 2.02 | retinaldehyde-binding parathyroid hormone-regulted protein IMAGE EST {IMAGE: 478711} |
| 654 | 2.02 | ADP-ribosylation factor (LAK) IMAGE EST {IMAGE: 387594} |
| 655 | 2.02 | IMAGE EST {IMAGE: 746888} |
| 656 | 2.02 | acetylcholine-interacting channel transcription factor IMAGE EST {IMAGE: 474184} |
| 657 | 2.02 | IMAGE EST {IMAGE: 423531} |
| 658 | 2.02 | ESTs, Moderately similar to ACYL-COA-BINDING PROTEIN [Bos taurus] {IMAGE: 463575} |
| 659 | 2.02 | similar to cab-1 gene (chlorophyll a/b-binding protein) ESTs {IMAGE: 948588} |
| 660 | 2.01 | IMAGE EST {IMAGE: 464866} |
| 661 | 2.01 | NAPOR neuroblastoma SH—SY-5y apoptosis RNA-binding protein IMAGE EST {IMAGE: 597832} |
| 662 | 2.01 | ESTs, Weakly similar to MATRIN 3 [R. norvegicus] {IMAGE: 876381} |
| 663 | 2.01 | ESTs {IMAGE: 905429} |
| 664 | 2.01 | neuronal calcium sensor-1 IMAGE EST {IMAGE: 401764} |
| 665 | 2.00 | GlaNAc-T2 sialytransferase IMAGE EST {IMAGE: 347921} |
| 666 | 2.00 | Protein kinase, cAMP dependent regulatory, type I beta {IMAGE: 736724} |
| 667 | 2.00 | GABA receptor IMAGE EST {IMAGE: 618375} |
| 668 | 2.00 | RNA stability IMAGE EST {IMAGE: 402843} |
| 669 | 2.00 | nucleolysin Tia-1 IMAGE EST {IMAGE: 751577} |
| 670 | 2.00 | ESTs, Moderately similar to golgi peripheral membrane protein p65 [R. norvegicus] {IMAGE: 677054} |
| 671 | 2.00 | liprin tyrosine phosphatase (LAR)-interacting protein IMAGE EST {IMAGE: 695231} |
| 672 | 2.00 | Mouse putative guanylate binding protein mRNA, complete cds {IMAGE: 576022} |
| 673 | 2.00 | regulator of G-protein signaling 12 IMAGE EST {IMAGE: 536574} |
| 674 | 0.51 | brain-specific p25 alpha CGI-38 protein homolog containing huntingtin regulatory element IMAGE EST |
| | | {IMAGE: 888544} |
| 675 | 0.50 | KIAA0098 chaperonin-containing TCP-1 epsilon protein folding subunit IMAGE EST {IMAGE: |
| | | 576003} |
| 676 | 0.50 | mitotic control protein IMAGE EST {IMAGE: 642330} |
| 677 | 0.50 | DIFF6 PROTEIN {IMAGE: 334138} |
| 678 | 0.50 | proliferation-associated mago-nashi protein homolog IMAGE EST {IMAGE: 751348} |
| 679 | 0.50 | KIAA1007 RE1-silencing Zn-finger transcription factor IMAGE EST {IMAGE: 479576} |
| 680 | 0.50 | TYRO3 protein tyrosine kinase 3 {IMAGE: 351163} |
| 681 | 0.50 | ESTs, Highly similar to ATP SYNTHASE F CHAIN, MITOCHONDRIAL [M. musculus] {IMAGE: |
| | | 403660} |
| 682 | 0.50 | bTF2 35 kDA subunit IMAGE EST {IMAGE: 671530} |
| 683 | 0.50 | NAD(P)H menadione oxidoreductase 1, dioxin inducible {IMAGE: 532186} |
| 684 | 0.50 | antisense to S-AdMet decarboxylase, c-Abl, Munc18-1, MHC locus IMAGE EST {IMAGE: 934425} |
| 685 | 0.50 | Zn-finger FIZ1-B splice variant IMAGE EST {IMAGE: 476367} |
| 686 | 0.50 | ESTs, Highly similar to HYPOTHETICAL 29.2 KD PROTEIN IN PHD1-PTM1 INTERGENIC |
| | | REGION [Saccharomyces cerevisiae] {IMAGE: 736297} |
| 687 | 0.50 | SPARC-related gene IMAGE EST {IMAGE: 425523} |
| 688 | 0.50 | IMAGE EST {IMAGE: 636193} |
| 689 | 0.50 | ESTs, Highly similar to HYPOTHETICAL 51.6 KD PROTEIN F59B2.5 IN CHROMOSOME III |
| | | [Caenorhabditis elegans] {IMAGE: 833508} |
| 690 | 0.50 | ESTs, Moderately similar to cell cycle progression 2 protein [H. sapiens] {IMAGE: 656956} |
| 691 | 0.50 | M. musculus mRNA for calpain-like protease {IMAGE: 733856} |
| 692 | 0.50 | ESTs, Weakly similar to No definition line found [C. elegans] {IMAGE: 820430} |
| 693 | 0.50 | DRIM (down-regulated in metastasis) IMAGE EST {IMAGE: 616975} |
| 694 | 0.50 | ESTs, Moderately similar to 26 S protease subunit 5b {IMAGE: 418563} |
| 695 | 0.50 | KIAA0402/pericentrin contains element common in erbB2/erbB3 ESTs {IMAGE: 640026} |
| 696 | 0.50 | KIAA1007 RE1-silencing transcription factor IMAGE EST {IMAGE: 948662} |
| 697 | 0.50 | IMAGE EST {IMAGE: 894275} |
| 698 | 0.50 | ESTs {IMAGE: 737800} |
| 699 | 0.50 | Early growth response 1 {IMAGE: 833284} |
| 700 | 0.50 | Mus musculus calmodulin-binding protein SHA1 (Sha1) mRNA, complete cds {IMAGE: 791460} |
| 701 | 0.50 | NUCLEOSOME ASSEMBLY PROTEIN 1-LIKE 1 {IMAGE: 671801} |
| 702 | 0.50 | ESTs, Highly similar to ELONGATION FACTOR G, MITOCHONDRIAL PRECURSOR [Rattus |
| | | norvegicus] {IMAGE: 889470} |
| 703 | 0.50 | chromodomain helicase DNA-binding protein IMAGE EST {IMAGE: 472805} |
| 704 | 0.50 | ESTs {IMAGE: 597797} |
| 705 | 0.50 | IMAGE EST {IMAGE: 425442} |
| 706 | 0.50 | pyridoxine 5′-phospho-oxidase IMAGE EST {IMAGE: 438286} |
| 707 | 0.50 | IMAGE EST {IMAGE: 424618} |
| 708 | 0.50 | ESTs, Highly similar to hypothetical protein, 100 K [R. norvegicus] {IMAGE: 808777} |
| 709 | 0.50 | Mus musculus RNaseP protein p30 (Rpp30) mRNA, complete cds {IMAGE: 389279} |
| 710 | 0.50 | Mouse mRNA for RPA16, complete cds {IMAGE: 425081} |
| 711 | 0.50 | receptor IMAGE EST {IMAGE: 749766} |
| 712 | 0.50 | SNF2L global transcription activator IMAGE EST {IMAGE: 791058} |
| 713 | 0.50 | nuclear SURF-6 protein IMAGE EST {IMAGE: 699378} |
| 714 | 0.50 | ESTs, Weakly similar to HYPOTHETICAL 11.4 KD PROTEIN C13G6.04 IN CHROMOSOME I |
| | | [Schizosaccharomyces pombe] {IMAGE: 579733} |
| 715 | 0.49 | tumour suppressing subtransferable candidate 1, RB-associated p48-like protein IMAGE EST {IMAGE: |
| | | 640062} |
| 716 | 0.49 | fetal JUMONJI protein IMAGE EST {IMAGE: 761554} |
| 717 | 0.49 | ESTs, Moderately similar to progesterone receptor-related protein p23 [H. sapiens] {IMAGE: 618630} |
| 718 | 0.49 | KIAA0011 general transcription factor IIIC polypeptide 2 beta subunit 110 kDa IMAGE EST {IMAGE: |
| | | 390118} |
| 719 | 0.49 | casein kinase II alpha chain ESTs {IMAGE: 735147} |
| 720 | 0.49 | Mus musculus mRNA for G-protein coupled receptor kinase 6-B {IMAGE: 640178} |
| 721 | 0.49 | IMAGE EST {IMAGE: 534015} |
| 722 | 0.49 | tyrosine protein kinase associated protein IMAGE EST {IMAGE: 423081} |
| 723 | 0.49 | ESTs, Weakly similar to KIAA0154 gene product is related to mouse gamma adaptin. [H. sapiens] |
| | | {IMAGE: 400308} |
| 724 | 0.49 | ligase/peptidase IMAGE EST {IMAGE: 751619} |
| 725 | 0.49 | ESTs, Highly similar to SET PROTEIN [Homo sapiens] {IMAGE: 780092} |
| 726 | 0.49 | Dipeptidase 1 (renal) {IMAGE: 316237} |
| 727 | 0.49 | IMAGE EST {IMAGE: 476022} |
| 728 | 0.49 | intermediary metabolism ESTs {IMAGE: 597201} |
| 729 | 0.49 | neuronal pentraxin receptor IMAGE EST {IMAGE: 352795} |
| 730 | 0.49 | INS-1 winged helix M-phase phosphoprotein IMAGE EST {IMAGE: 465555} |
| 731 | 0.49 | HSPC005 hu Chr. 11 fetal spleen ESTs {IMAGE: 807462} |
| 732 | 0.49 | mitogen-activated protein kinase IMAGE EST {IMAGE: 762210} |
| 733 | 0.49 | Glycerol phosphate dehydrogenase 1, mitochondrial {IMAGE: 351221} |
| 734 | 0.49 | alternatively spliced brain transcript IMAGE EST {IMAGE: 472664} |
| 735 | 0.49 | IMAGE EST {IMAGE: 400408} |
| 736 | 0.49 | IMAGE EST {IMAGE: 350881} |
| 737 | 0.49 | ESTs {IMAGE: 424559} |
| 738 | 0.48 | IMAGE EST {IMAGE: 573595} |
| 739 | 0.48 | GART and AML locus of human Chr. 21q22.1 ESTs {IMAGE: 699377} |
| 740 | 0.48 | Mus musculus mRNA for protein L. partial cds {IMAGE: 386234} |
| 741 | 0.48 | Mus musculus U4/U6 snRNP 90 kDa protein gene, complete cds {IMAGE: 576991} |
| 742 | 0.48 | ESTs {IMAGE: 317597} |
| 743 | 0.48 | Rac GTPase-activating protein IMAGE EST {IMAGE: 577676} |
| 744 | 0.48 | ESTs, Highly similar to ATP SYNTHASE LIPID-BINDING PROTEIN P2 PRECURSOR [Rattus |
| | | norvegicus] {IMAGE: 698109} |
| 745 | 0.48 | IMAGE EST {IMAGE: 437685} |
| 746 | 0.48 | T-complex testis expressed 1 {IMAGE: 762306} |
| 747 | 0.48 | mitochondrial ATP-binding protein IMAGE EST {IMAGE: 354969} |
| 748 | 0.48 | Mus musculus carbonic anhydrase IV gene, complete cds {IMAGE: 332285} |
| 749 | 0.48 | mitochondrial hydroxymethylglutaryl-CoA synthase IMAGE EST {IMAGE: 464392} |
| 750 | 0.48 | IMAGE EST {IMAGE: 406706} |
| 751 | 0.48 | AT-rich ESTs {IMAGE: 804212} |
| 752 | 0.48 | IMAGE EST {IMAGE: 581115} |
| 753 | 0.48 | IMAGE EST {IMAGE: 315536} |
| 754 | 0.48 | IMAGE EST {IMAGE: 639975} |
| 755 | 0.48 | HSPC007 splice variant homolog IMAGE EST {IMAGE: 580166} |
| 756 | 0.48 | cytokine remodeling-associated protein IMAGE EST {IMAGE: 482432} |
| 757 | 0.48 | neuronal apotosis-inhibiting nuclear receptor transcription activator IMAGE EST {IMAGE: 697208} |
| 758 | 0.47 | Lamin B2 {IMAGE: 478334} |
| 759 | 0.47 | ESTs, Weakly similar to PROBABLE RNA HELICASE IN WAPA-LICT INTERGENIC REGION |
| | | [Bacillus subtilis] {IMAGE: 403381} |
| 760 | 0.47 | IMAGE EST {IMAGE: 779373} |
| 761 | 0.47 | IMAGE EST {IMAGE: 441227} |
| 762 | 0.47 | IMAGE EST {IMAGE: 746908} |
| 763 | 0.47 | core-binding factor alpha subunit 2 MTGR1a (MTG-8-like protein) ESTs {IMAGE: 775188} |
| 764 | 0.47 | ESTs, Highly similar to P80-COILIN [Homo sapiens] {IMAGE: 351869} |
| 765 | 0.47 | ESTs, Highly similar to ARGINYL-TRNA SYNTHETASE [Cricetulus longicaudatus] {IMAGE: |
| | | 439640} |
| 766 | 0.47 | hormone-releasing factor IMAGE EST {IMAGE: 620209} |
| 767 | 0.47 | Syntrophin, acidic 1 {IMAGE: 318957} |
| 768 | 0.47 | DNA polymerase delta small subunit IMAGE EST {IMAGE: 468823} |
| 769 | 0.47 | ESTs {IMAGE: 618926} |
| 770 | 0.47 | cleavage and polyadenylation specificity factor IMAGE EST {IMAGE: 353445} |
| 771 | 0.47 | Mus musculus Btk locus, complete cds {IMAGE: 920442} |
| 772 | 0.47 | ESTs {IMAGE: 443441} |
| 773 | 0.47 | brain region-specific adenylate kinase 4 IMAGE EST {IMAGE: 403946} |
| 774 | 0.47 | Mus musculus elongation factor 1-beta homolog mRNA, complete cds {IMAGE: 442662} |
| 775 | 0.47 | Endoglin {IMAGE: 484299} |
| 776 | 0.47 | inhibin-related protein IMAGE EST {IMAGE: 639902} |
| 777 | 0.47 | melavonate pyrophosphate decarboxylase IMAGE EST {IMAGE: 479201} |
| 778 | 0.47 | Ig heavy chain IMAGE EST {IMAGE: 961056} |
| 779 | 0.47 | ESTs, Highly similar to CYCLIN-DEPENDENT KINASES REGULATORY SUBUNIT 1 [Homo |
| | | sapiens] {IMAGE: 961282} |
| 780 | 0.47 | KIAA0664 Zn-finger transglutaminase IMAGE EST {IMAGE: 373505} |
| 781 | 0.47 | ring finger protein 4 IMAGE EST {IMAGE: 894169} |
| 782 | 0.47 | Importin beta {IMAGE: 419193} |
| 783 | 0.47 | receptor channel protein IMAGE EST {IMAGE: 720904} |
| 784 | 0.47 | EYA4 gene for eyes absent ESTs {IMAGE: 671169} |
| 785 | 0.47 | IMAGE EST {IMAGE: 920335} |
| 786 | 0.47 | ESTs {IMAGE: 638129} |
| 787 | 0.46 | DNA damage tolerance protein IMAGE EST {IMAGE: 535949} |
| 788 | 0.46 | Fibroblast inducible secreted protein {IMAGE: 336325} |
| 789 | 0.46 | IMAGE EST {IMAGE: 577766} |
| 790 | 0.46 | M. musculus AP-2.2 gene {IMAGE: 419886} |
| 791 | 0.46 | contains neuronal survivor genes'' regulatory elements (apoptosis-inhibitory) ESTs {IMAGE: 750205} |
| 792 | 0.46 | mitochondrial cyclophilin IMAGE EST {IMAGE: 483584} |
| 793 | 0.46 | protein kinase A anchor protein 95 IMAGE EST {IMAGE: 523965} |
| 794 | 0.46 | ESTs {IMAGE: 439665} |
| 795 | 0.46 | phosphatidylglycerophosphate synthase IMAGE EST {IMAGE: 642201} |
| 796 | 0.46 | IMAGE EST {IMAGE: 633284} |
| 797 | 0.46 | ESTs, Highly similar to TRANSCRIPTION FACTOR BTF3 [Homo sapiens] {IMAGE: 692936} |
| 798 | 0.46 | DNA-binding protein IMAGE EST {IMAGE: 572463} |
| 799 | 0.46 | receptor IMAGE EST {IMAGE: 581799} |
| 800 | 0.46 | Mus musculus G protein-coupled receptor mRNA, complete cds {IMAGE: 493202} |
| 801 | 0.46 | IMAGE EST {IMAGE: 465848} |
| 802 | 0.46 | Enabled homolog (Drosophila) {IMAGE: 653622} |
| 803 | 0.46 | Mus musculus mRNA for pyruvate kinase, complete cds {IMAGE: 873690} |
| 804 | 0.46 | ESTs, Highly similar to transcription factor NF-AT 45K chain [H. sapiens] {IMAGE: 368164} |
| 805 | 0.46 | ESTs {IMAGE: 581834} |
| 806 | 0.46 | IMAGE EST {IMAGE: 837565} |
| 807 | 0.46 | Mouse mRNA for ras-GTPase-activating protein SH3-domain binding protein, complete cds {IMAGE: |
| | | 423772} |
| 808 | 0.46 | seven transmembrane GPCR IMAGE EST {IMAGE: 747167} |
| 809 | 0.46 | interleukin enhancer binding factor IMAGE EST {IMAGE: 571805} |
| 810 | 0.46 | annexin homolog IMAGE EST {IMAGE: 476275} |
| 811 | 0.46 | cdc25A ESTs {IMAGE: 641051} |
| 812 | 0.46 | integral membrane gp210 nuclear pore protein IMAGE EST {IMAGE: 355445} |
| 813 | 0.46 | heomeobox containing-like protein methylase IMAGE EST {IMAGE: 818653} |
| 814 | 0.46 | Mus musculus mRNA for protein L, partial cds {IMAGE: 933939} |
| 815 | 0.46 | ESTs, Highly similar to ELONGATION FACTOR 1-GAMMA [Homo sapiens] {IMAGE: 671075} |
| 816 | 0.46 | ESTs, Highly similar to nucleoporin Nup84 [R. norvegicus] {IMAGE: 643755} |
| 817 | 0.46 | ESTs, Weakly similar to C08F8.2 [C. elegans] {IMAGE: 577312} |
| 818 | 0.46 | IMAGE EST {IMAGE: 439620} |
| 819 | 0.46 | M. musculus mRNA for high mobility group 2 protein {IMAGE: 747136} |
| 820 | 0.46 | ribose phosphate phosphokinase I ESTs {IMAGE: 679015} |
| 821 | 0.46 | oocyte-specific protein P100 IMAGE EST {IMAGE: 445310} |
| 822 | 0.46 | Mus musculus rho7 (rho7) mRNA, partial cds {IMAGE: 385746} |
| 823 | 0.46 | ESTs, Highly similar to MITOCHONDRIAL ELONGATION FACTOR TS PRECURSOR [Homo |
| | | sapiens] {IMAGE: 699237} |
| 824 | 0.45 | outer mitochondrial membrane protein translocase 34 kDa IMAGE EST {IMAGE: 693114} |
| 825 | 0.45 | aldehyde dehyrogenase E3 isozyme IMAGE EST {IMAGE: 572614} |
| 826 | 0.45 | retinal protein IMAGE EST {IMAGE: 482138} |
| 827 | 0.45 | kinase-interacting protein IMAGE EST {IMAGE: 317894} |
| 828 | 0.45 | Mouse mRNA for RNA polymerase II subuunit RPB14, complete cds {IMAGE: 636513} |
| 829 | 0.45 | ribosomal protein L39 IMAGE EST {IMAGE: 620239} |
| 830 | 0.45 | IMAGE EST {IMAGE: 443413} |
| 831 | 0.45 | M. musculus mRNA for Pr22 protein {IMAGE: 716630} |
| 832 | 0.45 | Nedd-4-like ubiquitin protein ligase regulator of Notch signaling pathway IMAGE EST {IMAGE: |
| | | 354910} |
| 833 | 0.45 | IMAGE EST {IMAGE: 580810} |
| 834 | 0.45 | M. musculus mRNA for mSUG1 {IMAGE: 480685} |
| 835 | 0.45 | L-asparaginase IMAGE EST {IMAGE: 550910} |
| 836 | 0.45 | Mus musculus CDK-activating kinase assembly factor p36/MAT1, complete cds {IMAGE: 480395} |
| 837 | 0.45 | IMAGE EST {IMAGE: 871585} |
| 838 | 0.45 | SH2-containing Nsp2 antiestrogen resistance 3 protein IMAGE EST {IMAGE: 465403} |
| 839 | 0.45 | Mus musculus mRNA for 26S proteasome non-ATPase subunit {IMAGE: 747059} |
| 840 | 0.45 | neurotropin-3 homolog IMAGE EST {IMAGE: 760832} |
| 841 | 0.45 | KIAA0518 IMAGE EST {IMAGE: 424329} |
| 842 | 0.45 | IT12 in Huntingon's disease region IMAGE EST {IMAGE: 834148} |
| 843 | 0.45 | ESTs, Weakly similar to PROBABLE eIF 3 RNA-BINDING SUBUNIT [C. elegans] {IMAGE: 571406} |
| 844 | 0.45 | CCAAT/enhancer binding protein alpha (C/EBP), related sequence 1 {IMAGE: 680123} |
| 845 | 0.45 | decarboxylase/oxidase ESTs {IMAGE: 805129} |
| 846 | 0.45 | IMAGE EST {IMAGE: 576901} |
| 847 | 0.45 | KIAA0595 variant IMAGE EST {IMAGE: 336684} |
| 848 | 0.45 | IMAGE EST {IMAGE: 577010} |
| 849 | 0.45 | IMAGE EST {IMAGE: 733738} |
| 850 | 0.45 | Replication factor C, 140 kDa {IMAGE: 420302} |
| 851 | 0.45 | ESTs {IMAGE: 720951} |
| 852 | 0.45 | GTP phosphate-binding protein IMAGE EST {IMAGE: 421129} |
| 853 | 0.45 | basic transcription element binding protein BTEB-1 IMAGE EST {IMAGE: 472332} |
| 854 | 0.45 | cdc-homolog IMAGE EST {IMAGE: 550649} |
| 855 | 0.44 | similar to MBP ESTs {IMAGE: 775875} |
| 856 | 0.44 | ADP-ribosylation factor IMAGE EST {IMAGE: 476610} |
| 857 | 0.44 | ESTs {IMAGE: 580935} |
| 858 | 0.44 | Secreted phosphoprotein 1 {IMAGE: 571759} |
| 859 | 0.44 | Cell division cycle 25A {IMAGE: 441394} |
| 860 | 0.44 | vilin-like tumour suppressor metastasis protein IMAGE EST {IMAGE: 763352} |
| 861 | 0.44 | IMAGE EST {IMAGE: 573743} |
| 862 | 0.44 | hyrolase protease IMAGE EST {IMAGE: 473802} |
| 863 | 0.44 | Phosphoribosylglycinamide formyltransferase {IMAGE: 535188} |
| 864 | 0.44 | Regulatory factor (trans-acting) 2 {IMAGE: 388321} |
| 865 | 0.44 | Enolase 3. beta muscle {IMAGE: 316967} |
| 866 | 0.44 | cardiolipin synthetase IMAGE EST {IMAGE: 314456} |
| 867 | 0.44 | insulin receptor substrate ATP binding protein IMAGE EST {IMAGE: 318263} |
| 868 | 0.44 | Mus musculus 26S proteasome ATPase (CIP21) mRNA, complete cds {IMAGE: 466254} |
| 869 | 0.44 | IMAGE EST {IMAGE: 775342} |
| 870 | 0.44 | Mus musculus mRNA for prefoldin subunit 2 {IMAGE: 748879} |
| 871 | 0.43 | EphB1 receptor tyrosine protein kinase (elk) IMAGE EST {IMAGE: 480205} |
| 872 | 0.43 | FSH regulatory domian IMAGE EST {IMAGE: 764803} |
| 873 | 0.43 | ribosomal protein L17 IMAGE EST {IMAGE: 481485} |
| 874 | 0.43 | intronless highly charged protein IMAGE EST {IMAGE: 437167} |
| 875 | 0.43 | UBIQUITIN-LIKE PROTEIN FUBI {IMAGE: 698100} |
| 876 | 0.43 | IMAGE EST {IMAGE: 618834} |
| 877 | 0.43 | ESTs, Highly similar to PROBABLE RIBOSOMAL PROTEIN B0303.15 IN CHROMOSOME III |
| | | [Caenorhabditis elegans] {IMAGE: 574981} |
| 878 | 0.43 | NGF-responsive SM-20 protein IMAGE EST {IMAGE: 876655} |
| 879 | 0.43 | metal response element binding protein IMAGE EST {IMAGE: 779876} |
| 880 | 0.43 | Mouse testis abundant mRNA sequence {IMAGE: 437994} |
| 881 | 0.43 | ESTs {IMAGE: 638265} |
| 882 | 0.43 | mitochondrial arginyl-tRNA synthetase precursor IMAGE EST {IMAGE: 576572} |
| 883 | 0.43 | KIAA0281 IMAGE EST {IMAGE: 464800} |
| 884 | 0.43 | melanoma antigen p15 IMAGE EST {IMAGE: 803541} |
| 885 | 0.43 | homeobox-containing infant brain replication factor IMAGE EST {IMAGE: 427295} |
| 886 | 0.43 | ESTs, Highly similar to KIAA0017 [H. sapiens] {IMAGE: 735617} |
| 887 | 0.43 | Splicing factor, arginine/serine-rich 3 (SRp20) {IMAGE: 873314} |
| 888 | 0.43 | suppressor of stem-loop protein IMAGE EST {IMAGE: 575067} |
| 889 | 0.43 | cytoskeletal kinase substrate IMAGE EST {IMAGE: 437626} |
| 890 | 0.43 | IMAGE EST {IMAGE: 353521} |
| 891 | 0.43 | similar to Ssu81p and ribosomal protein L17B ESTs {IMAGE: 642467} |
| 892 | 0.43 | p52 pro-apoptotic protein TNF-beta-like IMAGE EST {IMAGE: 775135} |
| 893 | 0.43 | Mus musculus MSL15 gene {IMAGE: 582028} |
| 894 | 0.43 | enoyl CoA hydratase/isomerase IMAGE EST {IMAGE: 523463} |
| 895 | 0.43 | Eph receptor A2 {IMAGE: 459170} |
| 896 | 0.43 | IMAGE EST {IMAGE: 367802} |
| 897 | 0.43 | IMAGE EST {IMAGE: 337217} |
| 898 | 0.43 | Mus musculus ribosomal protein L21 mRNA, complete cds {IMAGE: 748374} |
| 899 | 0.43 | Mouse mRNA for 8-oxo-dGTPase, complete cds {IMAGE: 737058} |
| 900 | 0.43 | IMAGE EST {IMAGE: 576686} |
| 901 | 0.43 | basic transcription element binding GC-box binding Zn-finger protein IMAGE EST {IMAGE: 874192} |
| 902 | 0.43 | Alkaline phosphatase 2, liver {IMAGE: 465052} |
| 903 | 0.43 | KIAA0156 scamous cell carcinoma antigen SART-3 IMAGE EST {IMAGE: 367601} |
| 904 | 0.43 | lysosomal pathway protein ESTs {IMAGE: 464548} |
| 905 | 0.43 | ESTs, Moderately similar to PUTATIVE MITOCHONDRIAL RIBOSOMAL PROTEIN S14 |
| | | [Caenorhabditis elegans] {IMAGE: 419614} |
| 906 | 0.42 | membrane protein IMAGE EST {IMAGE: 809055} |
| 907 | 0.42 | Mus musculus selenocysteine tRNA gene transcription activating factor (m-Staf) mRNA, complete cds |
| | | {IMAGE: 574939} |
| 908 | 0.42 | Lymphocyte protein tyrosine kinase {IMAGE: 550845} |
| 909 | 0.42 | nuclear protein IMAGE EST {IMAGE: 746994} |
| 910 | 0.42 | ESTs, Highly similar to SERINE HYDROXYMETHYLTRANSFERASE, MITOCHONDRIAL |
| | | [Oryctolagus cuniculus] {IMAGE: 676311} |
| 911 | 0.42 | claudin-6 four transmembrane component of tight junction IMAGE EST {IMAGE: 805419} |
| 912 | 0.42 | Heat shock protein, 74 kDa {IMAGE: 889543} |
| 913 | 0.42 | ESTs, Weakly similar to similar to peptidase family C19 [C. elegans] {IMAGE: 313741} |
| 914 | 0.42 | Mus musculus serine/threonine kinase (sak-a) mRNA, complete cds {IMAGE: 777598} |
| 915 | 0.42 | ESTs, Highly similar to PROBABLE PHOSPHOSERINE AMINOTRANSFERASE [Oryctolagus |
| | | cuniculus] {IMAGE: 573784} |
| 916 | 0.42 | ESTs, Highly similar to KIAA0398 [H. sapiens] {IMAGE: 721931} |
| 917 | 0.42 | hypothetical brain protein ESTs {IMAGE: 735607} |
| 918 | 0.42 | ESTs, Weakly similar to KIAA0005 [H. sapiens] {IMAGE: 532659} |
| 919 | 0.42 | Breast cancer 2 {IMAGE: 761119} |
| 920 | 0.42 | IMAGE EST {IMAGE: 653427} |
| 921 | 0.42 | ESTs, Weakly similar to T06D8.8 [C. elegans] {IMAGE: 354212} |
| 922 | 0.42 | protamine IMAGE EST {IMAGE: 746955} |
| 923 | 0.42 | TM receptor ESTs {IMAGE: 750782} |
| 924 | 0.42 | Protein tyrosine phosphatase, non-receptor type 2 {IMAGE: 437517} |
| 925 | 0.42 | ESTs, Highly similar to 40S RIBOSOMAL PROTEIN S4, X ISOFORM [Homo sapiens; Mus musculus] |
| | | {IMAGE: 425352} |
| 926 | 0.42 | tight junction zona occludens 2 protein (ZO-2) IMAGE EST {IMAGE: 427339} |
| 927 | 0.41 | Zn-finger protein IMAGE EST {IMAGE: 480747} |
| 928 | 0.41 | IMAGE EST {IMAGE: 466150} |
| 929 | 0.41 | IMAGE EST {IMAGE: 440985} |
| 930 | 0.41 | RAD51 associated protein 1 {IMAGE: 760795} |
| 931 | 0.41 | ESTs, Highly similar to EUKARYOTIC TRANSLATION INITIATION FACTOR 3 BETA SUBUNIT |
| | | [H. sapiens] {IMAGE: 820518} |
| 932 | 0.41 | IMAGE EST {IMAGE: 775596} |
| 933 | 0.41 | syntenic to GART-AML xanthine dehydrogenase locus IMAGE EST {IMAGE: 751500} |
| 934 | 0.41 | IMAGE EST {IMAGE: 439542} |
| 935 | 0.41 | IMAGE EST {IMAGE: 717469} |
| 936 | 0.41 | Mus musculus protein kinase C inhibitor (mPKCI) mRNA, complete cds {IMAGE: 533117} |
| 937 | 0.41 | KIAA0560 neuronal adaptin IMAGE EST {IMAGE: 478374} |
| 938 | 0.41 | IMAGE EST {IMAGE: 439902} |
| 939 | 0.41 | ESTs, Highly similar to CELLULAR APOPTOSIS SUSCEPTIBILITY PROTEIN [H. sapiens] {IMAGE: |
| | | 874148} |
| 940 | 0.41 | IMAGE EST {IMAGE: 752290} |
| 941 | 0.41 | IMAGE EST {IMAGE: 443711} |
| 942 | 0.41 | Plasminogen activator inhibitor, type II {IMAGE: 574835} |
| 943 | 0.41 | IMAGE EST {IMAGE: 408040} |
| 944 | 0.41 | protein phosphatase PP-X IMAGE EST {IMAGE: 476407} |
| 945 | 0.41 | Cell division cycle 25A {IMAGE: 851054} |
| 946 | 0.41 | IMAGE EST {IMAGE: 445127} |
| 947 | 0.41 | dehydrogenase/transferase IMAGE EST {IMAGE: 475443} |
| 948 | 0.41 | ESTs {IMAGE: 637237} |
| 949 | 0.41 | Murine Glvr-1 mRNA, complete cds {IMAGE: 620195} |
| 950 | 0.41 | IMAGE EST {IMAGE: 763563} |
| 951 | 0.41 | IMAGE EST {IMAGE: 572869} |
| 952 | 0.41 | ESTs {IMAGE: 640556} |
| 953 | 0.41 | IMAGE EST {IMAGE: 427480} |
| 954 | 0.41 | IMAGE EST {IMAGE: 831991} |
| 955 | 0.40 | ESTs {IMAGE: 418955} |
| 956 | 0.40 | tight junction zona occludens ZO-2 protein IMAGE EST {IMAGE: 419848} |
| 957 | 0.40 | bisphosphate 3′ nucleotidase 1 lithium-sensitive IMAGE EST {IMAGE: 439411} |
| 958 | 0.40 | Mus musculus elongation factor 1-beta homolog mRNA, complete cds {IMAGE: 733810} |
| 959 | 0.40 | ESTs, Weakly similar to HYPOTHETICAL 6.3 KD PROTEIN ZK652.2 IN CHROMOSOME III |
| | | [C. elegans] {IMAGE: 385341} |
| 960 | 0.40 | translocation/splicing/rearrangement ‘hot spot’ IMAGE EST {IMAGE: 445230} |
| 961 | 0.40 | ESTs, Highly similar to INTERFERON-RELATED PROTEIN PC4 [Mus musculus] {IMAGE: 387482} |
| 962 | 0.40 | ESTs, Highly similar to ELONGATION FACTOR 1-DELTA [Homo sapiens] {IMAGE: 442585} |
| 963 | 0.40 | Mus musculus UNC-51-like kinase ULK1 (Ulk1) mRNA, complete cds {IMAGE: 438917} |
| 964 | 0.40 | IMAGE EST {IMAGE: 406351} |
| 965 | 0.40 | ESTs, Highly similar to PUTATIVE ASPARAGINYL-TRNA SYNTHETASE DED81 [Saccharomyces |
| | | cerevisiae] {IMAGE: 805842} |
| 966 | 0.40 | mitochondrial ribosomal protein S5 IMAGE EST {IMAGE: 574832} |
| 967 | 0.40 | ESTs {IMAGE: 642145} |
| 968 | 0.40 | lipoic acid synthetase precursor IMAGE EST {IMAGE: 525104} |
| 969 | 0.40 | ESTs, Highly similar to PUTATIVE ADENOSINE KINASE [Saccharomyces cerevisiae] {IMAGE: |
| | | 748517} |
| 970 | 0.40 | Transformation related protein 53 {IMAGE: 409069} |
| 971 | 0.40 | Mus musculus SH3-containing protein SH3P4 mRNA, complete cds {IMAGE: 352836} |
| 972 | 0.40 | T-LYMPHOCYTE ACTIVATED PROTEIN {IMAGE: 472605} |
| 973 | 0.40 | transcription factor IMAGE EST {IMAGE: 635025} |
| 974 | 0.40 | IMAGE EST {IMAGE: 804482} |
| 975 | 0.40 | ESTs {IMAGE: 386101} |
| 976 | 0.40 | homeobox protein Cup9 IMAGE EST {IMAGE: 474339} |
| 977 | 0.40 | adult testis protein IMAGE EST {IMAGE: 598898} |
| 978 | 0.40 | heme oxygenase-2 ESTs {IMAGE: 478167} |
| 979 | 0.40 | TGF-beta1 masking protein large subunit precursor IMAGE EST {IMAGE: 733726} |
| 980 | 0.40 | ESTs {IMAGE: 407997} |
| 981 | 0.40 | bystin ce411 adhesion troponin and cytokeratin IMAGE EST {IMAGE: 475670} |
| 982 | 0.40 | Flap structure specific endonuclease 1 {IMAGE: 467501} |
| 983 | 0.39 | Mus musculus SIK similar protein mRNA, complete cds {IMAGE: 643463} |
| 984 | 0.39 | ribosomal protein L13A IMAGE EST {IMAGE: 574251} |
| 985 | 0.39 | fetal heart protein IMAGE EST {IMAGE: 476205} |
| 986 | 0.39 | Notch gene homolog 4, (Drosophila) {IMAGE: 457150} |
| 987 | 0.39 | NADH-ubiquinone oxidoreductase subunit B14.5A IMAGE EST {IMAGE: 465903} |
| 988 | 0.39 | Mus musculus mRNA for ribosomal protein L35a {IMAGE: 659928} |
| 989 | 0.39 | ESTs, Moderately similar to KIAA0253 [H. sapiens] {IMAGE: 573609} |
| 990 | 0.39 | Mus musculus maf10 (mAF10) mRNA, complete cds {IMAGE: 736069} |
| 991 | 0.39 | DNA helicase IMAGE EST {IMAGE: 720870} |
| 992 | 0.39 | growth arrest differentiation induced gene IMAGE EST {IMAGE: 468633} |
| 993 | 0.39 | KIAA0136 IMAGE EST {IMAGE: 575554} |
| 994 | 0.39 | GTP-binding protein IMAGE EST {IMAGE: 640177} |
| 995 | 0.39 | IMAGE EST {IMAGE: 834934} |
| 996 | 0.39 | IMAGE EST {IMAGE: 400741} |
| 997 | 0.39 | Gar1 low molecular weight neurofilament APP-binding protein IMAGE EST {IMAGE: 475814} |
| 998 | 0.39 | ESTs {IMAGE: 808377} |
| 999 | 0.39 | IMAGE EST {IMAGE: 583869} |
| 1000 | 0.39 | cytoplasmic C-1-tetrahydrofolate synthase IMAGE EST {IMAGE: 478383} |
| 1001 | 0.39 | Zn-finger protein RP-8 IMAGE EST {IMAGE: 763597} |
| 1002 | 0.39 | splicing protein (kidney) ESTs {IMAGE: 659193} |
| 1003 | 0.39 | Mus musculus gene for DJ-1, complete cds {IMAGE: 571278} |
| 1004 | 0.39 | mitotic spindle coiled-coil protein aldolase C IMAGE EST {IMAGE: 535199} |
| 1005 | 0.39 | ribosomal protein L17 IMAGE EST {IMAGE: 696858} |
| 1006 | 0.39 | Mus musculus putative histone deacetylase (HD1) mRNA, partial cds {IMAGE: 850354} |
| 1007 | 0.39 | ESTs, Highly similar to 40S RIBOSOMAL PROTEIN S15A [Homo sapiens; Rattus norvegicus] |
| | | {IMAGE: 466295} |
| 1008 | 0.39 | IMAGE EST {IMAGE: 619385} |
| 1009 | 0.39 | IMAGE EST {IMAGE: 425049} |
| 1010 | 0.39 | Epoxide hydrolase 2, cytoplasmic {IMAGE: 579349} |
| 1011 | 0.39 | ESTs, Highly similar to NUCLEAR TRANSPORT FACTOR 2 [Homo sapiens; Rattus norvegicus] |
| | | {IMAGE: 762516} |
| 1012 | 0.39 | intracellular hyaluronan receptor IMAGE EST {IMAGE: 676748} |
| 1013 | 0.39 | Acyl-CoA dehydrogenase, medium chain {IMAGE: 483333} |
| 1014 | 0.39 | ESTs, Highly similar to ARGINYL-TRNA SYNTHETASE [Cricetulus longicaudatus] {IMAGE: |
| | | 850426} |
| 1015 | 0.38 | cytokine receptor-like molecule IMAGE EST {IMAGE: 405779} |
| 1016 | 0.38 | ESTs, Highly similar to PROTEASOME 28 KD SUBUNIT 1 [Drosophila melanogaster] {IMAGE: |
| | | 651274} |
| 1017 | 0.38 | PET112 mitochondrial respiratory chain cytochrome C-oxidase regulating protein IMAGE EST {IMAGE: |
| | | 481576} |
| 1018 | 0.38 | IMAGE EST {IMAGE: 735773} |
| 1019 | 0.38 | M. musculus mRNA for mTGIF protein {IMAGE: 722623} |
| 1020 | 0.38 | Hexokinase 1 {IMAGE: 318642} |
| 1021 | 0.38 | nucleoporin p54 IMAGE EST {IMAGE: 595972} |
| 1022 | 0.38 | RAMP3 receptor activity modifying protein IMAGE EST {IMAGE: 761158} |
| 1023 | 0.38 | Mus musculus groucho-related gene 4 protein (Grg4) mRNA, partial cds {IMAGE: 949722} |
| 1024 | 0.38 | Glutathione-S-transferase, alpha 2 (Yc2) {IMAGE: 766582} |
| 1025 | 0.38 | NUCLEOSIDE DIPHOSPHATE KINASE A {IMAGE: 480329} |
| 1026 | 0.38 | oxidase serine/threonin protein kinase IMAGE EST {IMAGE: 465744} |
| 1027 | 0.38 | Fas-associated factor 1 {IMAGE: 481242} |
| 1028 | 0.38 | CGI-124 IMAGE EST {IMAGE: 577501} |
| 1029 | 0.38 | Mus musculus smooth muscle LIM protein (Crp2/SmLim) mRNA, complete cds {IMAGE: 763424} |
| 1030 | 0.38 | U1 AND U2 SMALL NUCLEAR RIBONUCLEOPROTEIN E {IMAGE: 408693} |
| 1031 | 0.38 | 7-3 K RNA polymerase III small cytoplasmic RNA 7SK IMAGE EST {IMAGE: 480196} |
| 1032 | 0.38 | alpha-tubulin IMAGE EST {IMAGE: 818256} |
| 1033 | 0.38 | cytokine-related protein IMAGE EST {IMAGE: 445439} |
| 1034 | 0.38 | IMAGE EST {IMAGE: 493683} |
| 1035 | 0.38 | IMAGE EST {IMAGE: 642165} |
| 1036 | 0.38 | structural protein IMAGE EST {IMAGE: 641368} |
| 1037 | 0.38 | alanine aminotransferase IMAGE EST {IMAGE: 425857} |
| 1038 | 0.38 | ESTs, Weakly similar to No definition line found [C. elegans] {IMAGE: 809206} |
| 1039 | 0.37 | IMAGE EST {IMAGE: 933183} |
| 1040 | 0.37 | IMAGE EST {IMAGE: 748639} |
| 1041 | 0.37 | ribosomal protein S3 ESTs {IMAGE: 747180} |
| 1042 | 0.37 | kinase locus IMAGE EST {IMAGE: 385358} |
| 1043 | 0.37 | IP3-5/6 kinase ESTs {IMAGE: 872986} |
| 1044 | 0.37 | ATP-binding IMAGE EST {IMAGE: 329816} |
| 1045 | 0.37 | repeat region IMAGE EST {IMAGE: 672656} |
| 1046 | 0.37 | IMAGE EST {IMAGE: 617288} |
| 1047 | 0.37 | IMAGE EST {IMAGE: 423327} |
| 1048 | 0.37 | ssDNA-binding mitochondrial protein precursor IMAGE EST {IMAGE: 459094} |
| 1049 | 0.37 | TFE3 transcriptional activator H-L-H & Leu zippper DNA-binding ESTs {IMAGE: 834917} |
| 1050 | 0.37 | receptor transcription factor IMAGE EST {IMAGE: 735089} |
| 1051 | 0.37 | RAB geranylgeranyl transferase, b subunit {IMAGE: 455483} |
| 1052 | 0.37 | KIAA0274 IMAGE EST {IMAGE: 479759} |
| 1053 | 0.37 | ESTs, Weakly similar to ZK856.11 [C. elegans] {IMAGE: 778381} |
| 1054 | 0.37 | Treacher Coffins Franceschetti syndrome 1, homolog {IMAGE: 473404} |
| 1055 | 0.37 | ESTs, Weakly similar to N2,N2-DIMETHYLGUANOSINE TRNA METHYLTRANSFERASE |
| | | PRECURSOR [S. cerevisiae] {IMAGE: 406889} |
| 1056 | 0.37 | ESTs, Highly similar to SMALL NUCLEAR RIBONUCLEOPROTEIN SM D2 [Homo sapiens] |
| | | {IMAGE: 351154} |
| 1057 | 0.37 | ALPHA-INTERNEXIN {IMAGE: 660756} |
| 1058 | 0.36 | ESTs, Weakly similar to Y53C12B.2 [C. elegans] {IMAGE: 678992} |
| 1059 | 0.36 | ESTs, Weakly similar to ORF YOR173w [S. cerevisiae] {IMAGE: 441983} |
| 1060 | 0.36 | MHC class III RD locus IMAGE EST {IMAGE: 483476} |
| 1061 | 0.36 | ESTs, Moderately similar to F28F8.3 [C. elegans] {IMAGE: 699466} |
| 1062 | 0.36 | uterus mRNA IMAGE EST {IMAGE: 464381} |
| 1063 | 0.36 | ESTs, Weakly similar to F28F8.3 [C. elegans] {IMAGE: 736584} |
| 1064 | 0.36 | FRUCTOSE-BISPHOSPHATE ALDOLASE A {IMAGE: 903419} |
| 1065 | 0.36 | Nuceoside phosphorylase {IMAGE: 607469} |
| 1066 | 0.36 | Mus musculus phosphomannomutase Sec53p homolog mRNA complete cds {IMAGE: 775067} |
| 1067 | 0.36 | ESTs, Weakly similar to HYPOTHETICAL 19.4 KD PROTEIN D2007.4 IN CHROMOSOME III |
| | | [C. elegans] {IMAGE: 803817} |
| 1068 | 0.36 | Carbon catabolite repression 4 homolog (S. cerevisiae) {IMAGE: 438774} |
| 1069 | 0.36 | sorting nexin 5 ESTs {IMAGE: 317976} |
| 1070 | 0.36 | regulator of pantophysin IMAGE EST {IMAGE: 903284} |
| 1071 | 0.36 | Msx-2 transcription factor homolog IMAGE EST {IMAGE: 791433} |
| 1072 | 0.36 | ESTs, Moderately similar to 60S ACIDIC RIBOSOMAL PROTEIN P2 [Homo sapiens] {IMAGE: |
| | | 476999} |
| 1073 | 0.36 | pescadillo ESTs {IMAGE: 477900} |
| 1074 | 0.36 | DEAD-box RNA helicase-like protein IMAGE EST {IMAGE: 749569} |
| 1075 | 0.36 | pituitary protein IMAGE EST {IMAGE: 481469} |
| 1076 | 0.36 | KIAA1089 IMAGE EST {IMAGE: 480026} |
| 1077 | 0.36 | NADH-ubiquinone oxidoreductase subunit B14.5A IMAGE EST {IMAGE: 386766} |
| 1078 | 0.36 | Mus musculus von Hippel-Lindau binding protein homolog (vbp-1) mRNA, complete cds {IMAGE: |
| | | 693287} |
| 1079 | 0.36 | Notchless modulator of Notch signaling IMAGE EST {IMAGE: 404268} |
| 1080 | 0.36 | centaurin-alpha IP4-PIP-binding Zn-finger GTPase-activating protein IMAGE EST {IMAGE: 386122} |
| 1081 | 0.36 | Antigen identified by monoclonal antibodies 4F2 {IMAGE: 478301} |
| 1082 | 0.36 | nuclear WD40-containing P-protein periodic tryptophan protein IMAGE EST {IMAGE: 576570} |
| 1083 | 0.35 | myosin heavy chain beta isoform IMAGE EST {IMAGE: 466413} |
| 1084 | 0.35 | IMAGE EST {IMAGE: 418473} |
| 1085 | 0.35 | Alcohol dehydrogenase 5 {IMAGE: 636207} |
| 1086 | 0.35 | IMAGE EST {IMAGE: 577180} |
| 1087 | 0.35 | Chaperonin subunit 4 (delta) {IMAGE: 459668} |
| 1088 | 0.35 | DNA primase, p49 subunit {IMAGE: 746463} |
| 1089 | 0.35 | Mus musculus mRNA for eIF3 p66. complete cds {IMAGE: 820355} |
| 1090 | 0.35 | IMAGE EST {IMAGE: 761127} |
| 1091 | 0.35 | ESTs {IMAGE: 467873} |
| 1092 | 0.35 | ESTs, Moderately similar to NUCLEAR PORE COMPLEX PROTEIN NUP107 [R. norvegicus] |
| | | {IMAGE: 777779} |
| 1093 | 0.35 | ESTs {IMAGE: 372421} |
| 1094 | 0.35 | ESTs, Highly similar to PROTEASOME ZETA CHAIN [Homo sapiens] {IMAGE: 572285} |
| 1095 | 0.35 | ESTs, Highly similar to PROTEASOME THETA CHAIN [Rattus norvegicus] {IMAGE: 571569} |
| 1096 | 0.35 | KIAA0017 IMAGE EST {IMAGE: 388936} |
| 1097 | 0.35 | KIAA0896 DD5 progestin-induced protein IMAGE EST {IMAGE: 633261} |
| 1098 | 0.35 | ESTs, Highly similar to herpesvirus associated ubiquitin-specific protease [H. sapiens] {IMAGE: 793038} |
| 1099 | 0.35 | ESTs, Highly similar to DEOXYHYPUSINE SYNTHASE [H. sapiens] {IMAGE: 736328} |
| 1100 | 0.35 | skin E17 DEEPEST coiled-coil protein IMAGE EST {IMAGE: 348123} |
| 1101 | 0.35 | calcium-binding, kinase receptor-like IMAGE EST {IMAGE: 482885} |
| 1102 | 0.35 | protease acetyl transferase ATP-binding IMAGE EST {IMAGE: 401741} |
| 1103 | 0.35 | Mus musculus epidermal growth factor receptor-binding protein GRB7 mRNA, complete cds {IMAGE: |
| | | 679007} |
| 1104 | 0.35 | ESTs, Weakly similar to ZK795.3 [C. elegans] {IMAGE: 717387} |
| 1105 | 0.35 | Protein-interacting protein ESTs {IMAGE: 721532} |
| 1106 | 0.35 | T-COMPLEX PROTEIN 1, BETA SUBUNIT {IMAGE: 439254} |
| 1107 | 0.34 | Enabled homolog (Drosophila) {IMAGE: 808083} |
| 1108 | 0.34 | Mus musculus branched-chain alpha-ketoacid dehydrogenase E1 beta-subunit mRNA sequence {IMAGE: |
| | | 476163} |
| 1109 | 0.34 | cytoskeletal Ig-like molecule IMAGE EST {IMAGE: 440715} |
| 1110 | 0.34 | Mus musculus FK506-binding protein 25 homolog (mFKBP25) mRNA, partial cds {IMAGE: 636888} |
| 1111 | 0.34 | ESTs, Highly similar to PROBABLE PHOSPHOSERINE AMINOTRANSFERASE [Oryctolagus |
| | | cuniculus] {IMAGE: 746853} |
| 1112 | 0.34 | IMAGE EST {IMAGE: 418606} |
| 1113 | 0.34 | ESTs, Moderately similar to motor protein [H. sapiens] {IMAGE: 336021} |
| 1114 | 0.34 | IMAGE EST {IMAGE: 403220} |
| 1115 | 0.34 | nuclear pore complex glycoprotein p62 ESTs {IMAGE: 524367} |
| 1116 | 0.34 | Na/K-transporting ATPase alpha1 chain IMAGE EST {IMAGE: 351288} |
| 1117 | 0.34 | ESTs {IMAGE: 401221} |
| 1118 | 0.34 | leucyl-tRNA synthetase IMAGE EST {IMAGE: 748061} |
| 1119 | 0.34 | ribonuclease P 40 kDa subunit IMAGE EST {IMAGE: 831799} |
| 1120 | 0.34 | Mus musculus putative histone deacetylase (HD1) mRNA, partial cds {IMAGE: 374877} |
| 1121 | 0.34 | Fas-associated factor 1 {IMAGE: 534132} |
| 1122 | 0.34 | HMG box endosomal protein TOM1 IMAGE EST {IMAGE: 574459} |
| 1123 | 0.34 | Heat shock protein, 74 kDa {IMAGE: 444027} |
| 1124 | 0.34 | origin of replication recognition complex subunit 6 (Orc6), DNA repair/cell cycle checkpoint gene ESTs |
| | | {IMAGE: 791176} |
| 1125 | 0.34 | contains leucine-rich heptad repeats expressed in proliferating tissue IMAGE EST {IMAGE: 421766} |
| 1126 | 0.34 | dimethylguanosinetRNA ethyltransferase precursor IMAGE EST {IMAGE: 423713} |
| 1127 | 0.33 | NY-REN-45 antigen K-channel protein in renal carconima IMAGE EST {IMAGE: 778447} |
| 1128 | 0.33 | IFN-related RNA-binding protein IMAGE EST {IMAGE: 467830} |
| 1129 | 0.33 | ESTs {IMAGE: 617197} |
| 1130 | 0.33 | Mus musculus (clone mcori-lck9) S29 ribosomal protein mRNA, complete cds {IMAGE: 793127} |
| 1131 | 0.33 | ESTs, Highly similar to EUKARYOTIC INITIATION FACTOR 4A-LIKE NUK-34 [Homo sapiens] |
| | | {IMAGE: 779315} |
| 1132 | 0.33 | pescadillo IMAGE EST {IMAGE: 427222} |
| 1133 | 0.33 | IMAGE EST {IMAGE: 807957} |
| 1134 | 0.33 | Promyelocytic leukemia {IMAGE: 820560} |
| 1135 | 0.33 | eukaryotic initiation factor 4A-I IMAGE EST {IMAGE: 571841} |
| 1136 | 0.33 | M-phase P-protein 10 IMAGE EST {IMAGE: 597896} |
| 1137 | 0.33 | DNA ligase I, ATP-dependent {IMAGE: 388245} |
| 1138 | 0.33 | ESTs, Weakly similar to CYSTATHIONINE GAMMA-LYASE [Rattus norvegicus] {IMAGE: 679235} |
| 1139 | 0.33 | IMAGE EST {IMAGE: 874541} |
| 1140 | 0.33 | E-cadherin-, ankyrin-, uvomorulin-like protein IMAGE EST {IMAGE: 762237} |
| 1141 | 0.33 | IMAGE EST {IMAGE: 401167} |
| 1142 | 0.33 | KIAA0314 IMAGE EST {IMAGE: 335017} |
| 1143 | 0.33 | ESTs, Weakly similar to F54B3.3 [C. elegans] {IMAGE: 401821} |
| 1144 | 0.33 | guanine nucleotide-binding GPCR-like protein IMAGE EST {IMAGE: 425452} |
| 1145 | 0.33 | arsenite-translocating (resistance) ATPase IMAGE EST {IMAGE: 676921} |
| 1146 | 0.33 | KIAA0039 opiate octopamine receptor IMAGE EST {IMAGE: 735374} |
| 1147 | 0.33 | ESTs, Highly similar to THREONYL-TRNA SYNTHETASE, CYTOPLASMIC [Homo sapiens] |
| | | {IMAGE: 480488} |
| 1148 | 0.33 | Glutamate dehydrogenase {IMAGE: 442843} |
| 1149 | 0.33 | ESTs, Moderately similar to PRE-MRNA SPLICING FACTOR SRP20 [Homo sapiens; Mus musculus] |
| | | {IMAGE: 406282} |
| 1150 | 0.33 | IMAGE EST {IMAGE: 619836} |
| 1151 | 0.33 | eukaryotic initiation factor 2 alpha kinase IMAGE EST {IMAGE: 440053} |
| 1152 | 0.33 | origin of replication recognition complex subunit 6 (Orc6) ESTs {IMAGE: 426529} |
| 1153 | 0.33 | ESTs, Highly similar to PROBABLE ATP-DEPENDENT TRANSPORTER YER036C [Saccharomyces |
| | | cerevisiae] {IMAGE: 406088} |
| 1154 | 0.33 | lysosomal protein ESTs {IMAGE: 440194} |
| 1155 | 0.32 | Mus musculus C-terminal binding protein 2 mRNA, complete cds {IMAGE: 467669} |
| 1156 | 0.32 | ESTs, Highly similar to HYPOTHETICAL 34.7 KD PROTEIN IN SPT10-GCD14 INTERGENIC |
| | | REGION [S. cerevisiae] {IMAGE: 406154} |
| 1157 | 0.32 | Adenine nucleotide translocator 2, fibroblast {IMAGE: 465520} |
| 1158 | 0.32 | bisphosphate 3′ nucleotidase 1 lithium-sensitive IMAGE EST {IMAGE: 439033} |
| 1159 | 0.32 | ESTs {IMAGE: 639451} |
| 1160 | 0.32 | ESTs {IMAGE: 355454} |
| 1161 | 0.32 | ESTs, Weakly similar to coded for by C. elegans cDNA yk157f8.5 [C. elegans] {IMAGE: 354155} |
| 1162 | 0.32 | LL56-APP region ESTs {IMAGE: 619401} |
| 1163 | 0.32 | ESTs {IMAGE: 480204} |
| 1164 | 0.32 | Cell division cycle control protein 2a {IMAGE: 468792} |
| 1165 | 0.32 | KIAA0956 IMAGE EST {IMAGE: 573582} |
| 1166 | 0.32 | Mus musculus mRNA for mB1m, complete cds {IMAGE: 464447} |
| 1167 | 0.32 | alanyl-tRNA synthetase IMAGE EST {IMAGE: 388091} |
| 1168 | 0.32 | NNX3 ESTs {IMAGE: 576353} |
| 1169 | 0.32 | NAD(H) isocitrate dehydrogenase alpha chain IMAGE EST {IMAGE: 475796} |
| 1170 | 0.32 | ESTs, Highly similar to CAD PROTEIN [Homo sapiens] {IMAGE: 834541} |
| 1171 | 0.32 | CD97 precursor IMAGE EST {IMAGE: 573301} |
| 1172 | 0.32 | Mus musculus putative endo/exonuclease MmMre11b (MmMRE11) mRNA, alternative splicing product, |
| | | complete cds {IMAGE: 524361} |
| 1173 | 0.31 | ESTs, Weakly similar to T13H5.5 [C. elegans] {IMAGE: 848702} |
| 1174 | 0.31 | ESTs, Highly similar to transcription factor E2F-4 [M. musculus] {IMAGE: 422188} |
| 1175 | 0.31 | bright and dead ringer homolog IMAGE EST {IMAGE: 422167} |
| 1176 | 0.31 | similar to KIAA0032 IMAGE EST {IMAGE: 596719} |
| 1177 | 0.31 | ESTs {IMAGE: 427594} |
| 1178 | 0.31 | IMAGE EST {IMAGE: 933852} |
| 1179 | 0.31 | similar to CGI-122 protein IMAGE EST {IMAGE: 407354} |
| 1180 | 0.31 | DNA polymerase delta catalytic chain IMAGE EST {IMAGE: 424021} |
| 1181 | 0.31 | pirin NF-1 interacting nuclear protein IMAGE EST {IMAGE: 332396} |
| 1182 | 0.31 | regulator of chromosome condensation IMAGE EST {IMAGE: 422799} |
| 1183 | 0.31 | ESTs, Moderately similar to HYPOTHETICAL 27.1 KD PROTEIN CCE1-CAP1 INTERGENIC |
| | | REGION [S. cerevisiae] {IMAGE: 440795} |
| 1184 | 0.31 | alkaline phosphatase IMAGE EST {IMAGE: 535409} |
| 1185 | 0.31 | arginine N-methyltransferase 3 IMAGE EST {IMAGE: 441235} |
| 1186 | 0.31 | centaurin alpha IMAGE EST {IMAGE: 437602} |
| 1187 | 0.31 | Mus musculus HS1-associating protein (mHAX-1s) mRNA, complete cds {IMAGE: 874591} |
| 1188 | 0.31 | ESTs {IMAGE: 583932} |
| 1189 | 0.31 | IMAGE EST {IMAGE: 640682} |
| 1190 | 0.31 | IMAGE EST {IMAGE: 580623} |
| 1191 | 0.31 | putative nucleotide (ATP) binding protein IMAGE EST {IMAGE: 679011} |
| 1192 | 0.31 | KIAA0088 IMAGE EST {IMAGE: 482910} |
| 1193 | 0.31 | chromatin assembly factor I p60 subunit containing WD repeats IMAGE EST {IMAGE: 373854} |
| 1194 | 0.31 | Mouse mRNA for RNA polymerase I 40 kD subunit, complete cds {IMAGE: 335746} |
| 1195 | 0.30 | Glyceraldehyde-3-phosphate dehydrogenase {IMAGE: 579715} |
| 1196 | 0.30 | ESTs, Highly similar to LYSYL-TRNA SYNTHETASE [Cricetulus longicaudatus] {IMAGE: 439548} |
| 1197 | 0.30 | DNA POLYMERASE ALPHA/PRIMASE ASSOCIATED SUBUNIT {IMAGE: 476123} |
| 1198 | 0.30 | ribosomal protein L7 IMAGE EST {IMAGE: 524442} |
| 1199 | 0.30 | DEAD-box protein DP103 putative helicase IMAGE EST {IMAGE: 533415} |
| 1200 | 0.30 | similar to CGI-07 protein IMAGE EST {IMAGE: 894135} |
| 1201 | 0.30 | ESTs, Weakly similar to C01F6.8 [C. elegans] {IMAGE: 422667} |
| 1202 | 0.30 | Mus musculus mRNA for rabkinesin-6 {IMAGE: 633209} |
| 1203 | 0.30 | ribosomal protein S6 modification protein IMAGE EST {IMAGE: 961094} |
| 1204 | 0.30 | ESTs, Highly similar to Cdc5-like protein [R. norvegicus] {IMAGE: 833661} |
| 1205 | 0.30 | situin type 1 ADP-ribosylase IMAGE EST {IMAGE: 385901} |
| 1206 | 0.30 | thymidylate kinase IMAGE EST {IMAGE: 760921} |
| 1207 | 0.30 | Mus musculus ribosomal protein S5 mRNA, complete cds {IMAGE: 775645} |
| 1208 | 0.30 | IMAGE EST {IMAGE: 388212} |
| 1209 | 0.30 | ESTs, Weakly similar to HYPOTHETICAL 47.8 KD PROTEIN B0280.9 IN CHROMOSOME III |
| | | [C. elegans] {IMAGE: 441968} |
| 1210 | 0.30 | ESTs, Moderately similar to This sequence is almost identical to HUMRSC433.D13632. [H. sapiens] |
| | | {IMAGE: 576156} |
| 1211 | 0.30 | IMAGE EST {IMAGE: 790122} |
| 1212 | 0.30 | PEPTIDYL-PROLYL CIS-TRANS ISOMERASE A {IMAGE: 920055} |
| 1213 | 0.30 | NAD isocitrate dehydrogenase IMAGE EST {IMAGE: 775189} |
| 1214 | 0.29 | 60S RIBOSOMAL PROTEIN L3 {IMAGE: 570533} |
| 1215 | 0.29 | Mus musculus ribosomal protein L8 (RPL8) mRNA, complete cds {IMAGE: 716896} |
| 1216 | 0.29 | NAD/ATP binding protein IMAGE EST {IMAGE: 550869} |
| 1217 | 0.29 | IMAGE EST {IMAGE: 792736} |
| 1218 | 0.29 | House mouse; Musculusdomesticus female mammary carcinoma mRNA for mCDC47, complete cds |
| | | {IMAGE: 948894} |
| 1219 | 0.29 | nucleolar protein p40 associated with proliferation IMAGE EST {IMAGE: 779257} |
| 1220 | 0.29 | steroid binding protein IMAGE EST {IMAGE: 765841} |
| 1221 | 0.29 | IMAGE EST {IMAGE: 355808} |
| 1222 | 0.29 | ESTs, Highly similar to kinesin-related protein 2 [R. norvegicus] {IMAGE: 437761} |
| 1223 | 0.28 | DNA primase, p58 subunit {IMAGE: 680147} |
| 1224 | 0.28 | receptor IMAGE EST {IMAGE: 352409} |
| 1225 | 0.28 | KIFC1 neural kinesin CHO2 antigen IMAGE EST {IMAGE: 388296} |
| 1226 | 0.28 | LAK-4p chemokine IMAGE EST {IMAGE: 387227} |
| 1227 | 0.28 | TUBULIN ALPHA-4 CHAIN {IMAGE: 329726} |
| 1228 | 0.28 | ESTs, Highly similar to herpesvirus associated ubiquitin-specific protease [H. sapiens] {IMAGE: 779598} |
| 1229 | 0.28 | KIAA0888 stress-induced Doc4 brain Ten-m/Odz IMAGE EST {IMAGE: 457567} |
| 1230 | 0.28 | transforming acidic coiled-coil containing protein 3 IMAGE EST {IMAGE: 407274} |
| 1231 | 0.28 | ESTs, Weakly similar to f254 [E. coli] {IMAGE: 314236} |
| 1232 | 0.28 | IMAGE EST {IMAGE: 834712} |
| 1233 | 0.28 | ESTs, Highly similar to ALPHA ENOLASE [Mus musculus] {IMAGE: 643854} |
| 1234 | 0.28 | IMAGE EST {IMAGE: 720554} |
| 1235 | 0.28 | nuclear import karyopherin beta 3/Ran GTP-binding protein 5 IMAGE EST {IMAGE: 387946} |
| 1236 | 0.28 | IMAGE EST {IMAGE: 478417} |
| 1237 | 0.28 | ESTs, Highly similar to 60S RIBOSOMAL PROTEIN L15 [Rattus norvegicus] {IMAGE: 532770} |
| 1238 | 0.28 | X-ray repair complementing defective repair in Chinese hamster cells 5 {IMAGE: 573725} |
| 1239 | 0.27 | KIAA0314 ESTs {IMAGE: 445567} |
| 1240 | 0.27 | IMAGE EST {IMAGE: 804963} |
| 1241 | 0.27 | Mus musculus Hsp70-related NST-1 (hsr.1) mRNA, complete cds {IMAGE: 574381} |
| 1242 | 0.27 | Lymphocyte protein tyrosine kinase {IMAGE: 438125} |
| 1243 | 0.27 | IMAGE EST {IMAGE: 372368} |
| 1244 | 0.27 | Mus musculus coding region determinant binding protein mRNA, complete cds {IMAGE: 535036} |
| 1245 | 0.27 | ESTs, Highly similar to 60S RIBOSOMAL PROTEIN L1A [Xenopus laevis] {IMAGE: 699417} |
| 1246 | 0.27 | Mouse mRNA for mMIS5, complete cds {IMAGE: 444918} |
| 1247 | 0.27 | Down syndrome critical region gene 2 ESTs {IMAGE: 775603} |
| 1248 | 0.27 | Mus musculus histone H2A.Z (H2A.Z) mRNA, complete cds {IMAGE: 831643} |
| 1249 | 0.27 | transferrin R IMAGE EST {IMAGE: 403681} |
| 1250 | 0.27 | ESTs, Weakly similar to HYPOTHETICAL 86.9 KD PROTEIN ZK945.3 IN CHROMOSOME II |
| | | [Caenorhabditis elegans] {IMAGE: 331974} |
| 1251 | 0.26 | Apurinic/apyrimidinic endonuclease {IMAGE: 331901} |
| 1252 | 0.26 | ESTs, Highly similar to putative ATP/GTP-binding protein [H. sapiens] {IMAGE: 642977} |
| 1253 | 0.26 | cyclin B1 EST {IMAGE: 836311} |
| 1254 | 0.26 | arsenate resistance protein ARS2 ESTs {IMAGE: 932884} |
| 1255 | 0.26 | Retinoic acid receptor, gamma {IMAGE: 439731} |
| 1256 | 0.26 | chromatin assembly factor-I p60 subunit IMAGE EST {IMAGE: 761060} |
| 1257 | 0.26 | ESTs, Moderately similar to PTTG gene product [R. norvegicus] {IMAGE: 678793} |
| 1258 | 0.26 | nuclear protein np95 proliferation associated IMAGE EST {IMAGE: 873758} |
| 1259 | 0.26 | U22 snoRNA host gene (UHG) gene ESTs {IMAGE: 482641} |
| 1260 | 0.26 | Fibroblast growth factor 15 {IMAGE: 479758} |
| 1261 | 0.26 | IMAGE EST {IMAGE: 467472} |
| 1262 | 0.26 | Mus musculus dihydroorotate dehydrogenase mRNA, partial cds {IMAGE: 692981} |
| 1263 | 0.26 | Mus musculus branched-chain amino acid aminotransferase (Eca40) mRNA, nuclear gene encoding |
| | | mitochondrial protein {IMAGE: 776036} |
| 1264 | 0.26 | RNA cyclase homolog IMAGE EST {IMAGE: 437831} |
| 1265 | 0.25 | Proliferating cell nuclear antigen {IMAGE: 579735} |
| 1266 | 0.25 | IMAGE EST {IMAGE: 467550} |
| 1267 | 0.25 | Mus musculus mRNA for nuclear protein SA3 {IMAGE: 493305} |
| 1268 | 0.25 | ESTs {IMAGE: 805386} |
| 1269 | 0.25 | MITOCHONDRIAL MATRIX PROTEIN P1 PRECURSOR {IMAGE: 633625} |
| 1270 | 0.25 | Mouse mRNA for KIFC1, complete cds {IMAGE: 426992} |
| 1271 | 0.25 | Early development regulator {IMAGE: 350848} |
| 1272 | 0.25 | serine/threonine protein kinase W IMAGE EST {IMAGE: 657961} |
| 1273 | 0.25 | Mus musculus Tcl1 oncogene mRNA, complete cds {IMAGE: 961083} |
| 1274 | 0.25 | Glutamate oxaloacetate transaminase 1, soluble {IMAGE: 481381} |
| 1275 | 0.24 | citrin calcium-dependant mitochondrial carrier protein IMAGE EST {IMAGE: 834225} |
| 1276 | 0.24 | ESTs, Highly similar to teg292 protein [M. musculus] {IMAGE: 407480} |
| 1277 | 0.24 | denentin 48 kDa/52 kDa subunits IMAGE EST {IMAGE: 422953} |
| 1278 | 0.24 | G2/MITOTIC-SPECIFIC CYCLIN B1 {IMAGE: 576166} |
| 1279 | 0.24 | putative ATP/GTP-binding protein IMAGE EST {IMAGE: 403005} |
| 1280 | 0.24 | RIBOSOMAL PROTEIN S6 KINASE II ALPHA 1 {IMAGE: 355755} |
| 1281 | 0.24 | Mus musculus myosin light chain 2 mRNA, complete cds {IMAGE: 466382} |
| 1282 | 0.24 | ESTs, Highly similar to 60S RIBOSOMAL PROTEIN L37A [Gallus gallus] {IMAGE: 466384} |
| 1283 | 0.24 | ESTs, Moderately similar to oriP binding protein [H. sapiens] {IMAGE: 424705} |
| 1284 | 0.24 | calpain p94 large isoform IMAGE EST {IMAGE: 574491} |
| 1285 | 0.23 | Mus musculus ribosomal protein S5 mRNA, complete cds {IMAGE: 696769} |
| 1286 | 0.23 | pescadillo ESTs {IMAGE: 477828} |
| 1287 | 0.23 | ESTs {IMAGE: 805046} |
| 1288 | 0.23 | RNA polymerase III IMAGE EST {IMAGE: 441327} |
| 1289 | 0.22 | IMAGE EST {IMAGE: 331792} |
| 1290 | 0.22 | IMAGE EST {IMAGE: 420278} |
| 1291 | 0.22 | developmental kinase 2 tyrosine protein kinase IMAGE EST {IMAGE: 426239} |
| 1292 | 0.22 | Poly (ADP-ribose) polymerase {IMAGE: 466241} |
| 1293 | 0.22 | IMAGE EST {IMAGE: 917265} |
| 1294 | 0.22 | Mus musculus asparagine synthetase mRNA, complete cds {IMAGE: 337748} |
| 1295 | 0.22 | IMAGE EST {IMAGE: 577488} |
| 1296 | 0.22 | mitotic checkpoint protein kinase BUB1B ESTs {IMAGE: 465804} |
| 1297 | 0.22 | NAD-DEPENDENT METHYLENETETRAHYDROFOLATE DEHYDROGENASE {IMAGE: |
| | | 406031} |
| 1298 | 0.22 | CCAAT-enhancer binding protein C/EBP IMAGE EST {IMAGE: 738252} |
| 1299 | 0.21 | Protamine 3 {IMAGE: 400035} |
| 1300 | 0.21 | mitosis-specific chromosome segregation protein SMC1 IMAGE EST {IMAGE: 791570} |
| 1301 | 0.21 | ESTs {IMAGE: 574520} |
| 1302 | 0.21 | IMAGE EST {IMAGE: 803622} |
| 1303 | 0.20 | orip-binding protein/kinesin-like IMAGE EST {IMAGE: 438886} |
| 1304 | 0.20 | L1 cam region IMAGE EST {IMAGE: 734665} |
| 1305 | 0.20 | Cadherin 1 {IMAGE: 776133} |
| 1306 | 0.20 | Peroxisomal sarcosine oxidase {IMAGE: 790721} |
| 1307 | 0.20 | ribosomal protein L15 ESTs {IMAGE: 464084} |
| 1308 | 0.20 | Selenophosphate synthetase 2 {IMAGE: 791719} |
| 1309 | 0.20 | early transposon Etn/mitochondrial selenoprotein IMAGE EST {IMAGE: 722631} |
| 1310 | 0.20 | IMAGE EST {IMAGE: 805579} |
| 1311 | 0.20 | Mus musculus E1B 19K/Bcl-2-binding protein homolog (Nip3) mRNA, nuclear gene encoding |
| | | mitochondrial protein, complete cds {IMAGE: 571367} |
| 1312 | 0.20 | growth-related protein IMAGE EST {IMAGE: 575434} |
| 1313 | 0.19 | Mus musculus clone L8 variable group of 2-cell-stage gene family mRNA, complete cds {IMAGE: |
| | | 793218} |
| 1314 | 0.19 | cystatothionine gamma-lyase IMAGE EST {IMAGE: 525119} |
| 1315 | 0.19 | hKIAA0030 gene/minichromosome deficient/CDC L1/replication licencing factor IMAGE EST {IMAGE: |
| | | 441229} |
| 1316 | 0.19 | kruppel-like factor IMAGE EST {IMAGE: 444844} |
| 1317 | 0.19 | E1B 19K/Bcl-2-B homolog (Nip3)/calbindin-like IMAGE EST {IMAGE: 463388} |
| 1318 | 0.18 | KIAA0116/homolog of NK receptor IMAGE EST {IMAGE: 533085} |
| 1319 | 0.18 | pyroline-5-carboxylate reductase IMAGE EST {IMAGE: 402730} |
| 1320 | 0.18 | Mouse mRNA for mCDC46 protein, complete cds {IMAGE: 466047} |
| 1321 | 0.18 | beta-mannosidase precursor IMAGE EST {IMAGE: 641507} |
| 1322 | 0.18 | mismatch repair protein MutS (E. coli) homolog 2 {IMAGE: 945359} |
| 1323 | 0.18 | IMAGE EST {IMAGE: 765272} |
| 1324 | 0.18 | IMAGE EST {IMAGE: 456342} |
| 1325 | 0.18 | histone acetyl transferase IMAGE EST {IMAGE: 314146} |
| 1326 | 0.18 | ESTs {IMAGE: 316914} |
| 1327 | 0.18 | ESTs, Highly similar to AMIDOPHOSPHORIBOSYLTRANSFERASE PRECURSOR [Rattus |
| | | norvegicus] {IMAGE: 779343} |
| 1328 | 0.18 | lysophosphatydic acid phosphatase ESTs {IMAGE: 582668} |
| 1329 | 0.17 | IMAGE EST {IMAGE: 313254} |
| 1330 | 0.17 | ESTs, Weakly similar to PHF1 [H. sapiens] {IMAGE: 945218} |
| 1331 | 0.17 | contains regulatory elements IMAGE EST {IMAGE: 403663} |
| 1332 | 0.17 | Cyclin E {IMAGE: 833511} |
| 1333 | 0.17 | TRANSCRIPTION FACTOR S-II-RELATED PROTEIN {IMAGE: 748744} |
| 1334 | 0.16 | nucleolar repeats protein/ribosome biogenesis ESTs, Weakly similar to K07C5.4 [C. elegans] {IMAGE: |
| | | 933491} |
| 1335 | 0.16 | contains regulatory elements found in ribosomal RNAs IMAGE EST {IMAGE: 575290} |
| 1336 | 0.15 | G2/MITOTIC-SPECIFIC CYCLIN B1 {IMAGE: 751977} |
| 1337 | 0.15 | replication factor C (activator 1) IMAGE EST {IMAGE: 572970} |
| 1338 | 0.14 | Mus musculus very-long-chain acyl-CoA synthetase (VLCS) mRNA, complete cds {IMAGE: 572379} |
| 1339 | 0.14 | Phosphoribosylglycinamide formyltransferase {IMAGE: 476629} |
| 1340 | 0.14 | Phosphoribosylglycinamide formyltransferase {IMAGE: 477593} |
| 1341 | 0.14 | Mouse chromatin nonhistone high mobility group protein (HGM-I(Y), complete cds {IMAGE: 920268} |
| 1342 | 0.13 | equilibrative nucleoside transporter IMAGE EST {IMAGE: 805549} |
| 1343 | 0.13 | equilibrative nucleoside transporter IMAGE EST {IMAGE: 680512} |
| 1344 | 0.13 | contains spectrin elements IMAGE EST {IMAGE: 403735} |
| 1345 | 0.13 | IMAGE EST {IMAGE: 693719} |
| 1346 | 0.12 | Hemopoietic cell kinase {IMAGE: 638455} |
| 1347 | 0.12 | Carbonic anhydrase 2 {IMAGE: 579391} |
| 1348 | 0.12 | IMAGE EST {IMAGE: 402162} |
| 1349 | 0.12 | JM1 protein IMAGE EST {IMAGE: 833919} |
| 1350 | 0.12 | glycoamino acid (cationic) transporter IMAGE EST {IMAGE: 776058} |
| 1351 | 0.12 | Glutathione-S-transferase, alpha 1 (Ya) {IMAGE: 367627} |
| 1352 | 0.11 | KIAA0245/glycoprotein-associated amino acid (cationic) transporter IMAGE EST {IMAGE: 457955} |
| 1353 | 0.10 | asialoglycoprotein receptor 1 IMAGE EST {IMAGE: 949512} |
| 1354 | 0.10 | ESTs, Weakly similar to HYPOTHETICAL 15.4 KD PROTEIN C16C10.11 IN CHROMOSOME III |
| | | [C. elegans] {IMAGE: 334182} |
| 1355 | 0.08 | IMAGE EST {IMAGE: 893933} |
| 1356 | 0.07 | RAR-alpha IMAGE EST {IMAGE: 820336} |
| 1357 | 0.07 | KIAA0888 IMAGE EST {IMAGE: 442396} |
| 1358 | 0.07 | contains regulatory element of TFs (also is in HIV envelope glycoprotein) IMAGE EST {IMAGE: |
| | | 442966} |
| 1359 | 0.06 | Myeloblastosis oncogene-like 2 {IMAGE: 833557} |
| 1360 | 0.06 | CYP3A1 gene 5′ GRE responsive element ESTs {IMAGE: 791079} |
| 1361 | 0.06 | F-Boc protein FBX15 ubiquitin ligase regulator ESTs {IMAGE: 833477} |
| 1362 | 0.04 | Mouse mRNA for uridine phosphorylase, complete cds {IMAGE: 445862} |
| 1363 | 0.03 | ETn transposon/TGR gamma locus IMAGE EST {IMAGE: 535794} |
| 1364 | 0.02 | ETn transposon IMAGE EST {IMAGE: 633484} |
| 1365 | 0.01 | ESTs, Highly similar to retinoic acid-regulated protein pH 34 [M. musculus] {IMAGE: 944963} |

TABLE 2

| | IMAGE | | | | | |
|---|---|---|---|---|---|---|
| SEQ | Clone | GenBank | Template | SEQ | e | |
| ID NO | ID | ID | ID | ID NO | value | Description |
| 1 | 463135 | g4059261 | 407392.37 | 2022 | 0 | Human mRNA for IGF-II precursor (insulin-like growth factor). |
| 2 | 671661 | g1872629 | | | | |
| 3 | 464598 | g1497693 | | | | |
| 4 | 427016 | g4271958 | 380283.5 | 1976 | 0 | Human mRNA for KIAA0287 gene, partial cds. |
| 5 | 874833 | g2200656 | 222420.1 | 1653 | 0 | Human extracellular matrix protein 1 mRNA, complete cds. |
| 6 | 420322 | g1539066 | 245004.8 | 1771 | 0 | Human mRNA for integrin beta 1 subunit. |
| 7 | 478168 | g1529387 | | | | |
| 8 | 639481 | g1794338 | 806690.12 | 2164 | 0 | Human mRNA for insulin-like growth factor binding protein-3. |
| 9 | 766482 | g1918383 | 199977.8 | 1607 | 0 | Human retinal short-chain dehydrogenase/reductase retSDR1 mRNA, |
| | | | | | | complete cds. |
| 10 | 678765 | g1861400 | 222420.2 | 1654 | 0 | Human extracellular matrix protein 1 mRNA, alternative splice |
| | | | | | | variant, complete cds. |
| 11 | 425866 | g1436827 | 233180.14 | 1679 | 0 | Human P311 Human (3.1) mRNA, complete cds. |
| 12 | 532350 | g1564784 | 021677.7 | 1379 | 0 | Human clone JkA10 mRNA induced upon T-cell activation, 3′ end. |
| 13 | 374970 | g1369558 | 233180.1 | 1678 | 0 | Human P311 Human (3.1) mRNA, complete cds. |
| 14 | 422746 | g4199189 | | | | |
| 15 | 535652 | g1662749 | 235164.4c | 1713 | 0 | Human mRNA for amyloid A4(751) protein. |
| 16 | 694987 | g1827280 | 480898.39 | 2127 | 0 | Human glia-derived nexin (GDN) mRNA, 5′ end. |
| 17 | 464497 | g1504426 | 480898.39 | 2127 | 0 | Human glia-derived nexin (GDN) mRNA, 5′ end. |
| 18 | 418952 | g1542308 | | | | |
| 19 | 671910 | g1873455 | | | | |
| 20 | 367780 | g1355004 | 334852.5 | 1880 | 0 | Human transforming growth factor-beta-2 mRNA, complete cds. |
| 21 | 352450 | g1315693 | 229471.5 | 1660 | 0 | Human mRNA for collagen VI alpha-1 C-terminal globular domain. |
| 22 | 334132 | g1290623 | 229471.5 | 1660 | 0 | Human mRNA for collagen VI alpha-1 C-terminal globular domain. |
| 23 | 313322 | g1284409 | 1071415.1 | 1417 | 0 | Human IGF-I mRNA for insulin-like growth factor I. |
| 24 | 536526 | g4272801 | 429727.5 | 2060 | 0 | Human fibrillin-2 mRNA, complete cds. |
| 25 | 423028 | g4199193 | 201738.5 | 1622 | 0 | Human alpha-1 type XI collagen (COL11A1) mRNA, complete cds. |
| 26 | 463046 | g1493665 | | | | |
| 27 | 672201 | g1853656 | 347789.24 | 1924 | 0 | Human alpha-B-crystallin gene, 5′ end. |
| 28 | 407068 | g1541149 | 1078208.1 | 1468 | 0 | Human mRNA for carboxypeptidase E (EC 3.4.17.10). |
| 29 | 482955 | g1554683 | 201079.1 | 1614 | 0 | Human full length insert cDNA clone ZD42A08. |
| 30 | 402738 | g1391198 | 285031.4 | 1837 | 0 | Human collagenase type IV mRNA, 3′ end. |
| 31 | 831964 | g2157430 | 1078609.1 | 1470 | 0 | |
| 32 | 718665 | g1901788 | 256147.56 | 1815 | 0 | Human aorta caldesmon mRNA, complete cds. |
| 33 | 733420 | g1896293 | 233180.16 | 1680 | 0 | Human P311 Human (3.1) mRNA, complete cds. |
| 34 | 747101 | g1896969 | 1186377.1 | 1533 | 0 | Human mRNA for calpain-like protease CANPX. |
| 35 | 734305 | g1905506 | | | | |
| 36 | 468900 | g1509125 | 1166388.1 | 1512 | 0 | |
| 37 | 536306 | g1595337 | 257184.4 | 1819 | 6.00E−96 | Human proalpha 1 (I) chain of type I procollagen mRNA (partial). |
| 38 | 401608 | g1388876 | | | | |
| 39 | 426890 | g1446690 | 001821.6 | 1367 | 9.00E−05 | out at first |
| 40 | 580753 | g1715174 | | | | |
| 41 | 385914 | g5600038 | 256147.57 | 1816 | 0 | Human caldesmon mRNA, complete cds. |
| 42 | 401456 | g1436746 | | | | |
| 43 | 465620 | g1504196 | | | | |
| 44 | 620221 | g1759210 | | | | |
| 45 | 426546 | g1445943 | 413309.18c | 2041 | 0 | Human endonexin II mRNA, complete cds. |
| 46 | 457000 | g1487481 | 1172250.1 | 1522 | 9.00E−85 | CGI-56 protein |
| 47 | 367660 | g1356852 | 1163661.1 | 1508 | 0 | Human mRNA for NFI/CAAT-binding transcription factor 5 (CTF5). |
| 48 | 353202 | g1325849 | 406252.1 | 2019 | 8.00E−11 | hypothetical protein |
| 49 | 657264 | g1865297 | 1109345.1 | 1492 | 0 | Human (chromosome X) glypican (GPC3) mRNA, complete cds. |
| 50 | 406708 | g4257399 | | | | |
| 51 | 481927 | g1553633 | 412793.4 | 2039 | 0 | Human nuclear factor I-B2 (NFIB2) mRNA, complete cds. |
| 52 | 479405 | g1528806 | 429727.5 | 2060 | 0 | Human fibrillin-2 mRNA, complete cds. |
| 53 | 463207 | g1497504 | | | | |
| 54 | 618681 | g1756233 | 763966.1 | 2154 | 6.00E−13 | Human mRNA for 80K-L protein, complete cds. |
| 55 | 403597 | g1539666 | 1071628.8 | 1424 | 0 | Human procollagen C-protease (pCP-2) mRNA, complete cds. |
| 56 | 463342 | g1494749 | 252081.2 | 1795 | 0 | Human antigen NY—CO-33 (NY—CO-33) mRNA, complete cds. |
| 57 | 552603 | g1642160 | 336956.3 | 1887 | 3.00E−05 | putative |
| 58 | 949246 | g2305969 | 000305.1 | 1366 | 7.00E−83 | Human mRNA for CDEP, complete cds. |
| 59 | 400864 | g1387841 | | | | |
| 60 | 424348 | g1428536 | 235222.8 | 1717 | 0 | Human clone 23625 mRNA sequence. |
| 61 | 315581 | g1284184 | 273994.3 | 1831 | 0 | Human U6 snRNA-associated Sm-like protein LSm6 mRNA, complete |
| | | | | | | cds. |
| 62 | 808000 | g2116364 | 244973.2 | 1770 | 0 | Human vascular endothelial cell growth factor 165 receptor/ |
| | | | | | | neuropilin (VEGF165) mRNA, complete cds. |
| 63 | 850078 | g2164301 | 1143218.1 | 1497 | 0 | |
| 64 | 876297 | g2203612 | | | | |
| 65 | 820167 | g2101964 | 1189716.1 | 1552 | 0 | Human mRNA for gig1 protein. |
| 66 | 427289 | g1446744 | | | | |
| 67 | 443865 | g4276693 | | | | |
| 68 | 478040 | g4061336 | 334442.1c | 1878 | 0 | Human bone morphogenetic protein-3b. |
| 69 | 762240 | g1917691 | | | | |
| 70 | 464828 | g1505067 | | | | |
| 71 | 479382 | g1528480 | 279312.2 | 1834 | 1.00E−50 | HUMMLC2At; Human; ; 593 base-pairs. |
| 72 | 764513 | g1912736 | 816464.4c | 2182 | 0 | Human NMDA receptor glutamate-binding chain (hnrgw) mRNA, |
| | | | | | | partial cds. |
| 73 | 314639 | g1286605 | 207184.1 | 1635 | 0 | Human glypican-4 (GPC4) mRNA, complete cds. |
| 74 | 315775 | g1284389 | 230128.15 | 1662 | 0 | Human gamma-filamin (ABPL) mRNA, complete cds. |
| 75 | 476509 | g1530317 | 350707.15 | 1948 | 0 | Human mRNA for KIAA1114 protein, complete cds. |
| 76 | 427319 | g1446754 | 347875.3 | 1927 | 0 | Human T-cell leukemia LERK-2 (EPLG2) mRNA, complete cds. |
| 77 | 427345 | g4271979 | | | | |
| 78 | 617200 | g4443013 | 429995.3 | 2063 | 0 | Human mRNA; cDNA DKFZp564P1664 (from clone DKFZp564P1664). |
| 79 | 482995 | g1554868 | 347084.1 | | | |
| 80 | 721944 | g4764924 | 817053.1 | 2183 | 0 | |
| 81 | 352804 | g1316581 | | | | |
| 82 | 333232 | g1294183 | 1072399.3 | 1434 | 0 | Human Bet1p homolog (hbet1) mRNA, complete cds. |
| 83 | 634580 | g1776249 | 049935.4 | 1396 | 6.00E−44 | Human mRNA; cDNA DKFZp564C0362 (from clone DKFZp564C0362); |
| | | | | | | complete cds. |
| 84 | 386417 | g1371945 | | | | |
| 85 | 890760 | g2248333 | 256147.46 | 1814 | 0 | Human mRNA for caldesmon, 3′ UTR. |
| 86 | 698181 | g1863194 | 1073009.7 | 1450 | 1.00E−97 | Human elastin mRNA, complete cds. |
| 87 | 422325 | g1427499 | 121289.1 | 1560 | 0 | Human IGF-II gene exon 1 for insulin-like growth factor II |
| | | | | | | located on chromosome 11. |
| 88 | 697010 | g1838485 | 459119.1 | 2084 | 4.00E−65 | Human transcobalamin II (TCII) mRNA, complete cds. |
| 89 | 680894 | g1882418 | 346219.2 | 1916 | 0 | Human GDNF receptor alpha mRNA, complete cds. |
| 90 | 572542 | g1661694 | 1094500.1 | 1487 | 7.00E−64 | be(a-2-microglobulin precursor |
| 91 | 902923 | g2258850 | | | | |
| 92 | 747941 | g1897446 | 1071968.1 | 1429 | 0 | Human mRNA; cDNA DKFZp434B2411 (from clone DKFZp434B2411). |
| 93 | 423995 | g4271836 | 003457.2c | 1369 | 0 | Human HOX-2.5 gene for homeodomain protein, partial. |
| 94 | 807978 | g2116358 | | | | |
| 95 | 385318 | g1371476 | 256147.58c | 1817 | 0 | Human aorta caldesmon mRNA, complete cds. |
| 96 | 352909 | g1316741 | | | | |
| 97 | 735413 | g1907885 | 198006.3 | 1596 | 5.00E−74 | SPARC-related protein |
| 98 | 746643 | g1904823 | 1084333.3 | 1475 | 0 | Human (clone FBK III 11c) protein-tyrosine kinase (DRT) mRNA, |
| | | | | | | complete cds. |
| 99 | 464995 | g4058663 | 235132.8 | 1710 | 0 | Human profilin II mRNA, complete cds. |
| 100 | 427642 | g4061601 | 235197.9c | 1716 | 0 | Human protein-tyrosine phosphatase (PTPase MEG2) mRNA, complete |
| | | | | | | cds. |
| 101 | 479367 | g4057838 | 350895.2 | 1950 | 0 | Human mRNA for B-HLH DNA binding protein. |
| 102 | 401288 | g1428210 | 018653.1c | 1377 | 0 | |
| 103 | 332477 | g1282620 | | | | |
| 104 | 581193 | g4061843 | 235132.8 | 1710 | 0 | Human profilin II mRNA, complete cds. |
| 105 | 465937 | g1504589 | 482006.1c | 2137 | 0 | Human homeobox protein (HOX-1.3) gene, complete cds. |
| 106 | 692257 | g1862269 | 203947.1 | 1631 | 7.00E−86 | Human mRNA for MIFR-2, complete cds. |
| 107 | 478504 | g4061265 | 1186377.1 | 1533 | 0 | Human mRNA for calpain-like protease CANPX. |
| 108 | 406897 | g1394776 | 763639.1 | 2153 | 1.00E−57 | Human cellular retinol-binding protein mRNA, complete cds. |
| 109 | 578299 | g1677813 | 814967.4 | 2175 | 0 | Human mRNA for KIAA0913 protein, partial cds. |
| 110 | 935557 | g2292046 | | | | |
| 111 | 419146 | g1542389 | | | | |
| 112 | 672424 | g1853328 | | | | |
| 113 | 746798 | g1896930 | 381400.4 | 1977 | 0 | Human mRNA for fibulin-4. |
| 114 | 330825 | g1288931 | | | | |
| 115 | 803251 | g2049321 | 1071520.6 | 1421 | 0 | Human mRNA for RNase 4, complete cds. |
| 116 | 573265 | g4199851 | 233238.3 | 1683 | 0 | Human annexin III (ANX3) gene, alternative exons 1a and 1b. |
| 117 | 523686 | g1564930 | | | | |
| 118 | 873058 | g2192338 | 899303.1 | 2197 | 0 | Human mRNA for actin-binding protein (filamin) (ABP-280). |
| 119 | 695687 | g1834285 | 1072969.1c | 1443 | 0 | Human mRNA for neutral calponin, complete cds. |
| 120 | 670344 | g1840495 | | | | |
| 121 | 481410 | g1554247 | 235885.3 | 1724 | 2.00E−40 | Human zinc-finger helicase (hZFH) mRNA, complete cds. |
| 122 | 619950 | g1759275 | 817871.1 | 2184 | 0 | |
| 123 | 367785 | g1355012 | | | | |
| 124 | 572510 | g4059890 | 248640.1 | 1789 | 0 | |
| 125 | 356215 | g1339194 | 407061.11 | 2021 | 0 | Human mRNA; cDNA DKFZp586N1922 (from clone DKFZp586N1922), |
| | | | | | | partial cds. |
| 126 | 775893 | g1919031 | 1015595.1 | 1411 | 1.00E−86 | Human 26-kDa cell surface protein TAPA-1 mRNA, complete cds. |
| 127 | 846536 | g2138471 | 218425.1 | 1643 | 0 | |
| 128 | 420641 | g1542150 | 028443.2 | 1385 | 0 | Human sarcosine dehydrogenase (SARDH) mRNA, complete cds. |
| 129 | 762558 | g1909824 | 429995.3 | 2063 | 0 | Human mRNA; cDNA DKFZp564P1664 (from clone DKFZp564P1664). |
| 130 | 478571 | g1530979 | | | | |
| 131 | 617884 | g1756440 | | | | |
| 132 | 406295 | g1540698 | 720026.1 | 2138 | 0 | SPI-2 serine protease inhibitor (AA 1-407) |
| 133 | 808226 | g2116510 | 221501.2 | 1648 | 0 | Human ncx1 gene (exon 12). |
| 134 | 765332 | g1915642 | | | | |
| 135 | 888553 | g2233628 | 253450.6 | 1800 | 0 | Human mRNA for hFat protein. |
| 136 | 746644 | g1904820 | 361221.17 | 1963 | 0 | Human lumican mRNA, complete cds. |
| 137 | 752144 | g1936155 | 257739.16 | 1824 | 0 | Human KIAA0438 mRNA, complete cds. |
| 138 | 888571 | g2233635 | | | | |
| 139 | 424433 | g1428492 | | | | |
| 140 | 401980 | g1387738 | 1071605.1 | 1423 | 4.00E−36 | Dlxin-1 |
| 141 | 734101 | g1905364 | 245184.4 | 1773 | 0 | Human transforming growth factor-beta induced gene product |
| | | | | | | (BIGH3) mRNA, complete cds. |
| 142 | 367915 | g1355557 | 343884.1 | | | |
| 143 | 480684 | g1558142 | 1073003.1 | 1447 | 0 | Human talin mRNA, complete cds. |
| 144 | 622914 | g1758938 | 235797.3 | 1722 | 0 | Human Ste-20 related kinase SPAK mRNA, complete cds. |
| 145 | 903370 | g4617450 | 435792.1 | 2069 | 0 | |
| 146 | 427360 | g1446763 | 233213.4 | 1682 | 0 | Human mRNA for actin-binding protein (filamin) (ABP-280). |
| 147 | 402800 | g1390976 | | | | |
| 148 | 762159 | g1917554 | 201589.1 | 1620 | 0 | |
| 149 | 762339 | g1913048 | | | | |
| 150 | 439108 | g1447298 | 300972.5 | 1844 | 0 | Human NMDA receptor glutamate-binding chain (hnrgw) mRNA, |
| | | | | | | partial cds. |
| 151 | 464060 | g1494424 | 1072990.18 | 1446 | 0 | Human 40-kDa keratin intermediate filament precursor gene. |
| 152 | 763103 | g1909660 | | | | |
| 153 | 535763 | g1659237 | 230555.19 | 1664 | 0 | Human HBV associated factor (XAP4) mRNA, complete cds. |
| 154 | 820204 | g4320880 | 1035647.1 | 1414 | 3.00E−70 | Human unknown mRNA. |
| 155 | 373000 | g1365741 | | | | |
| 156 | 402995 | g1539222 | | | | |
| 157 | 891284 | g2248735 | 039966.1 | 1392 | 0 | Human antigen NY—CO-41 (NY—CO-41) mRNA, partial cds. |
| 158 | 439758 | g4199231 | 903458.2 | 2213 | 0 | Human Hox2.2 gene for a homeobox protein. |
| 159 | 425409 | g4271890 | 415803.1 | 2045 | 0 | Human mRNA; cDNA DKFZp564B222 (from clone DKFZp564B222). |
| 160 | 334872 | g1292213 | 1073003.16 | 1448 | 0 | Human talin (TLN) gene, exons 2 through 48. |
| 161 | 388477 | g1379747 | | | | |
| 162 | 444059 | g1475791 | 253958.8 | 1803 | 0 | Human myosin alkali light chain (atrial) mRNA, complete cds. |
| 163 | 619970 | g4288890 | 384264.3 | 1978 | 0 | Human mRNA for frizzled-1, complete cds. |
| 164 | 421150 | g1428883 | 351451.22 | 1955 | 0 | Human CD24 signal transducer mRNA, complete cds and 3′ |
| | | | | | | region. |
| 165 | 420535 | g4199173 | 244218.3 | 1761 | 0 | Human v-erbA related ear-3 gene. |
| 166 | 405907 | g1540438 | 345141.2 | 1910 | 0 | Human transcription factor (HTF4) mRNA, complete cds. |
| 167 | 467560 | g1506739 | 343884.8c | 1900 | 0 | Human mRNA for drebrin E, complete cds. |
| 168 | 851638 | g2235691 | | | | |
| 169 | 735527 | g1908231 | 231011.5 | 1666 | 0 | Human mRNA for membrane glycoprotein M6, complete cds. |
| 170 | 422403 | g4275705 | | | | |
| 171 | 680455 | g1861199 | 481379.8c | 2132 | 1.00E−96 | Human mRNA fragment for receptor-like furin. |
| 172 | 419756 | g1538851 | | | | |
| 173 | 423593 | g4271827 | | | | |
| 174 | 408110 | g1538731 | 233213.4 | 1682 | 0 | Human mRNA for actin-binding protein (filamin) (ABP-280). |
| 175 | 573845 | g1676557 | 332667.1 | 1871 | 6.00E−48 | Human mRNA; cDNA DKFZp566I1247 (from clone DKFZp566I1247). |
| 176 | 427205 | g4271969 | 202094.4 | 1625 | 0 | |
| 177 | 467313 | g1506457 | 334685.2 | 1879 | 0 | N-cadherin [Human, umbilical vein endothelial cells, mRNA, |
| | | | | | | 4132 nt]. |
| 178 | 620546 | g1759518 | 481101.3 | 2130 | 0 | Human calmodulin-dependent protein phosphatase catalytic |
| | | | | | | subunit (PPP3CA) mRNA, complete cds and alternative exon. |
| 179 | 463249 | g1497515 | 246501.7 | 1784 | 0 | Human mRNA for PDGF receptor beta-like tumor suppressor |
| | | | | | | (PRLTS), complete cds. |
| 180 | 949663 | g2283348 | 1077587.1 | 1467 | 0 | |
| 181 | 747364 | g1904956 | | | | |
| 182 | 482198 | g1553597 | 198006.3 | 1596 | 5.00E−74 | SPARC-related protein |
| 183 | 317958 | g1285908 | | | | |
| 184 | 669969 | g1888270 | 201617.34 | 1621 | 0 | Human mRNA for calgizzarin, complete cds. |
| 185 | 736372 | g1908971 | 197979.3 | 1595 | 0 | Human mRNA for tenascin-C, 7560 bp. |
| 186 | 620101 | g1757223 | | | | |
| 187 | 777018 | g1919075 | 413348.3c | 2042 | 0 | Human mRNA for selenoprotein P. |
| 188 | 572601 | g1662879 | | | | |
| 189 | 331264 | g1288363 | 350895.2 | 1950 | 0 | Human mRNA for B-HLH DNA binding protein. |
| 190 | 478428 | g4408981 | 763607.1 | 2152 | 3.00E−44 | Human clone 24747 mRNA sequence. |
| 191 | 765338 | g4303241 | 238509.4 | 1743 | 0 | Human p63 mRNA for transmembrane protein. |
| 192 | 657528 | g1794823 | | | | |
| 193 | 425279 | g1435895 | | | | |
| 194 | 734718 | g1895583 | | | | |
| 195 | 426146 | g4061584 | 257213.5 | 1820 | 0 | Human mRNA for Hic-5 , partial cds. |
| 196 | 385763 | g4256625 | | | | |
| 197 | 426010 | g4199205 | | | | |
| 198 | 388233 | g4613963 | 438075.1 | 2071 | 0 | |
| 199 | 335572 | g1294552 | 242309.7 | 1754 | 0 | Human mRNA for dihydropyrimidinase related protein-3, complete |
| | | | | | | cds. |
| 200 | 479895 | g1531368 | 150575.11 | 1570 | 0 | Human platelet-derived growth factor receptor alpha (PDGFRA) |
| | | | | | | mRNA, complete cds. |
| 201 | 385581 | g1371846 | 246037.15 | 1781 | 0 | Human thyroid receptor interactor (TRIP7) mRNA, 3′ end of |
| | | | | | | cds. |
| 202 | 644907 | g1807566 | | | | |
| 203 | 482326 | g4273452 | | | | |
| 204 | 637078 | g4295283 | 793752.2 | 2161 | 0 | Human lectomedin-1 beta (LEC1) mRNA, complete cds. |
| 205 | 425777 | g1436662 | | | | |
| 206 | 400530 | g1387774 | 028505.3c | 1386 | 6.00E−72 | KIAA1236 protein |
| 207 | 634792 | g1726295 | | | | |
| 208 | 870973 | g2186637 | 903105.13 | 2208 | 1.00E−23 | Human Tigger1 transposable element, complete consensus |
| | | | | | | sequence. |
| 209 | 699236 | g1876263 | 481101.3 | 2130 | 0 | Human calmodulin-dependent protein phosphatase catalytic |
| | | | | | | subunit (PPP3CA) mRNA, complete cds and alternative exon. |
| 210 | 466591 | g1500922 | | | | |
| 211 | 676376 | g1806844 | 1073003.17 | 1449 | 0 | Human talin mRNA, complete cds. |
| 212 | 621555 | g1760122 | 399755.3 | 1991 | 5.00E−41 | Human mRNA; cDNA DKFZp586E171 (from clone DKFZp586E171). |
| 213 | 671377 | g1842477 | 1190120.1 | 1556 | 0 | Human ERF-1 mRNA 3′ end. |
| 214 | 833346 | g4614446 | 235885.17c | 1723 | 0 | Human CHD3 mRNA, complete cds. |
| 215 | 849762 | g2180916 | | | | |
| 216 | 331794 | g1282101 | 174291.1 | 1580 | 0 | |
| 217 | 775597 | g1918555 | 300160.24 | 1843 | 0 | Human mRNA for protein p68. |
| 218 | 480854 | g1558222 | | | | |
| 219 | 404044 | g1539942 | | | | |
| 220 | 404536 | g1540287 | | | | |
| 221 | 368189 | g1355133 | | | | |
| 222 | 318735 | g1316330 | 1116916.1 | 1493 | 1.00E−92 | Human Ca2+ ATPase of fast-twitch skeletal muscle |
| | | | | | | sarcoplasmic reticulum adult and neonatal isoforms |
| | | | | | | (ATP2A1) gene, exons 16 to 23 and complete cds. |
| 223 | 466292 | g1504813 | 233213.4 | 1682 | 0 | Human mRNA for actin-binding protein (filamin) |
| | | | | | | (ABP-280). |
| 224 | 652207 | g1814770 | | | | |
| 225 | 807727 | g2116322 | | | | |
| 226 | 720566 | g1895126 | 474310.28 | 2094 | 0 | Human complement C1r mRNA, complete cds. |
| 227 | 871674 | g2187238 | | | | |
| 228 | 777549 | g1932671 | 481101.3 | 2130 | 0 | Human calmodulin-dependent protein phosphatase catalytic |
| | | | | | | subunit (PPP3CA) mRNA, complete cds and alternative exon. |
| 229 | 763629 | g1929767 | 794126.3 | 2162 | 0 | Human mRNA for KIAA0992 protein, partial cds. |
| 230 | 803404 | g2201297 | 040428.5 | 1395 | 0 | Human Fritz mRNA, complete cds. |
| 231 | 466988 | g1506530 | | | | |
| 232 | 483148 | g4604003 | | | | |
| 233 | 480236 | g1550947 | 238085.1 | 1739 | 0 | Human mRNA; cDNA DKFZp586F2423 (from clone DKFZp586F2423). |
| 234 | 850280 | g2165165 | 351091.26 | 1952 | 0 | Human protocadherin gamma A11 (PCDH-gainma-A11) mRNA, complete |
| | | | | | | cds. |
| 235 | 315962 | g1283340 | | | | |
| 236 | 523713 | g1565101 | | | | |
| 237 | 681615 | g1861868 | 197599.5 | 1592 | 0 | Human mRNA for protein S. |
| 238 | 425344 | g4061570 | 234223.15 | 1702 | 0 | Human pro-alpha-1 (V) collagen mRNA, complete cds. |
| 239 | 492502 | g1642906 | 1071628.8 | 1424 | 0 | Human procollagen C-protease (pCP-2) mRNA, complete cds. |
| 240 | 407096 | g1394958 | | | | |
| 241 | 634233 | g1765588 | 422072.14 | 2048 | 0 | Human tyrosine kinase (Tnk1) mRNA, complete cds. |
| 242 | 478021 | g1529144 | 201589.1 | 1620 | 0 | |
| 243 | 660896 | g1840319 | | | | |
| 244 | 426070 | g4199206 | 429579.7 | 2057 | 0 | Human jun dimerization protein gene, partial cds; cfos gene, |
| | | | | | | complete cds; and unknown gene. |
| 245 | 693148 | g4315599 | 757518.3 | 2142 | 3.00E−58 | Xap89 protein |
| 246 | 762791 | g1909922 | 285031.17 | 1836 | 0 | Human collagenase type IV mRNA, 3′ end. |
| 247 | 465043 | g1505213 | | | | |
| 248 | 427469 | g1445795 | | | | |
| 249 | 334106 | g1537434 | | | | |
| 250 | 419544 | g4276184 | | | | |
| 251 | 479921 | g1531460 | | | | |
| 252 | 735227 | g1896055 | | | | |
| 253 | 618910 | g4060524 | 410917.7c | 2034 | 0 | Human LIPA gene, exon 10. |
| 254 | 426965 | g1446709 | 474736.8 | 2106 | 0 | Human E2IG1 (E2IG1) mRNA, complete cds. |
| 255 | 640951 | g1807777 | 720026.1 | 2138 | 0 | SPI-2 serine protease inhibitor (AA 1-407) |
| 256 | 677488 | g1811883 | | | | |
| 257 | 846064 | g2076514 | 400607.21 | 1997 | 5.00E−95 | Human peripheral myelin protein 22 (GAS3) mRNA, complete cds. |
| 258 | 330595 | g1288256 | | | | |
| 259 | 808829 | g2193674 | 403528.2c | 2010 | 0 | Human FZD4 mRNA for WNT receptor Frizzled-4, complete cds. |
| 260 | 464915 | g1500705 | | | | |
| 261 | 478336 | g1529432 | | | | |
| 262 | 876326 | g2203625 | 1010408.1 | 1409 | 0 | Human mRNA for cathepsin C. |
| 263 | 367947 | g1355764 | 233829.15 | 1694 | 0 | Human transcription enhancer factor-5 mRNA, complete cds. |
| 264 | 581835 | g1726103 | | | | |
| 265 | 333511 | g1315889 | 1071467.6 | 1419 | 0 | Human mRNA; cDNA DKFZp586J0917 (from clone DKFZp586J0917); |
| | | | | | | partial cds. |
| 266 | 475631 | g4273377 | 404183.1 | 2011 | 0 | Human voltage-dependent calcium channel alpha 1G subunit a |
| | | | | | | isoform (CACNA1G) mRNA, complete cds. |
| 267 | 643158 | g1776469 | | | | |
| 268 | 749482 | g1937342 | | | | |
| 269 | 480454 | g1551150 | 362662.1 | 1965 | 0 | |
| 270 | 760918 | g2039781 | | | | |
| 271 | 751020 | g4316657 | 726130.1 | 2139 | 0 | |
| 272 | 444388 | g4057723 | 1166856.2 | 1514 | 0 | Human mRNA for reticulocalbin, complete cds. |
| 273 | 751385 | g4316966 | 349742.3 | 1936 | 0 | Human transcription factor (MEF2) mRNA, complete cds. |
| 274 | 402348 | g1388926 | 765245.1 | 2156 | 6.00E−79 | Human mRNA for neutral calponin, complete cds. |
| 275 | 420553 | g1542095 | 888865.1 | 2185 | 0 | Human mRNA for KIAA1224 protein, partial cds. |
| 276 | 622257 | g1767769 | | | | |
| 277 | 920587 | g2288403 | 198830.1 | 1599 | 0 | |
| 278 | 696542 | g1863765 | | | | |
| 279 | 464575 | g1497689 | 233747.1 | 1692 | 0 | Human clone 24670 mRNA secpaence. |
| 280 | 354859 | g1330557 | 245181.3c | 1772 | 1.00E−95 | Human mRNA for MAP kinase activated protein kinase. |
| 281 | 439383 | g4315259 | | | | |
| 282 | 764649 | g1913111 | | | | |
| 283 | 315890 | g1286831 | | | | |
| 284 | 876698 | g2232417 | 300160.24 | 1843 | 0 | Human mRNA for protein p68. |
| 285 | 736854 | g1909620 | 256145.6 | 1813 | 1.00E−50 | Human UDP-glucose: glycogen 4-alpha-D-glycosytransferase mRNA, |
| | | | | | | complete cds. |
| 286 | 722330 | g1888849 | | | | |
| 287 | 444383 | g4272013 | 757587.1 | 2145 | 0 | Human checkpoint suppressor 1 mRNA, complete cds. |
| 288 | 890486 | g4373839 | 471800.1 | 2089 | 2.00E−85 | Human mRNA for fibroblast tropomyosin TM30 (pl). |
| 289 | 680191 | g1861942 | 762190.1 | 2151 | 0 | |
| 290 | 698175 | g1863483 | | | | |
| 291 | 922991 | g2249284 | 199986.1 | 1608 | 0 | Human mRNA for partial NOV/plexin-A1 protein. |
| 292 | 638401 | g1826001 | 1137704.1 | 1496 | 7.00E−74 | Human mRNA; cDNA DKFZp586P1622 (from clone DKFZp586P1622). |
| 293 | 385853 | g1369035 | | | | |
| 294 | 388617 | g1375661 | | | | |
| 295 | 444842 | g1478978 | 346516.2 | 1917 | 0 | Human mRNA for MSX-2, complete cds. |
| 296 | 316113 | g1283096 | 233338.1 | 1685 | 0 | Human intergenic DNA between SURF-2 and SURF-4. |
| 297 | 890932 | g2248891 | 337621.2 | 1891 | 0 | Human neuronal DHP-sensitive, voltage-dependent, calcium |
| | | | | | | channel alpha-2b subunit mRNA, complete cds. |
| 298 | 420591 | g1542116 | 336834.2 | 1886 | 7.00E−98 | Human clones 23549 and 23762 mRNA, complete cds. |
| 299 | 331768 | g4216904 | 371288.7 | 1971 | 0 | Human mRNA for dihydropyrimidinase related protein-2, complete |
| | | | | | | cds. |
| 300 | 864361 | g2180856 | | | | |
| 301 | 574070 | g1676562 | 1072938.4 | 1442 | 0 | Human lysosomal pepstatin insensitive protease (CLN2) mRNA, |
| | | | | | | complete cds. |
| 302 | 334291 | g1318252 | 144576.1c | 1568 | 1.00E−29 | Alg2 |
| 303 | 482677 | g1555797 | | | | |
| 304 | 524459 | g1575935 | | | | |
| 305 | 466781 | g1501117 | | | | |
| 306 | 418645 | g1426772 | | | | |
| 307 | 418766 | g1426920 | | | | |
| 308 | 680426 | g1861189 | 360570.1 | 1960 | 4.00E−16 | LZIP-1 and LZIP-2 |
| 309 | 776048 | g1918653 | 1072938.4 | 1442 | 0 | Human lysosomal pepstatin insensitive protease (CLN2) mRNA, |
| | | | | | | complete cds. |
| 310 | 464603 | g1497291 | | | | |
| 311 | 419265 | g1543811 | | | | |
| 312 | 406999 | g4257421 | | | | |
| 313 | 419455 | g4199165 | | | | |
| 314 | 390016 | g1380369 | | | | |
| 315 | 331599 | g1282077 | 269837.11 | 1828 | 0 | Human PIL protein mRNA, complete cds. |
| 316 | 693244 | g1882667 | | | | |
| 317 | 408747 | g1541642 | | | | |
| 318 | 775514 | g2041254 | | | | |
| 319 | 733937 | g1905138 | | | | |
| 320 | 777555 | g1919315 | 244823.1 | 1769 | 0 | Human mRNA for protein kinase Dyrk2. |
| 321 | 420049 | g1541974 | 221055.8 | 1646 | 0 | Human non-p53 regulated PA26-T1 nuclear protein (PA26) mRNA, |
| | | | | | | complete cds. |
| 322 | 720937 | g1904312 | | | | |
| 323 | 492514 | g4057921 | | | | |
| 324 | 337618 | g1294407 | 344617.7 | 1909 | 0 | Human mRNA for nel-related protein 2, complete cds. |
| 325 | 536634 | g4272822 | | | | |
| 326 | 459284 | g1497434 | 384264.3 | 1978 | 0 | Human mRNA for frizzled-1, complete cds. |
| 327 | 408381 | g1541463 | | | | |
| 328 | 420484 | g1542038 | 053059.6c | 1397 | 2.00E−05 | unknown protein |
| 329 | 458802 | g1488968 | 401593.14 | 2004 | 0 | Human quiescin (Q6) mRNA, complete cds. |
| 330 | 425742 | g1436545 | | | | |
| 331 | 932999 | g2284192 | 1071733.3 | 1427 | 0 | Human DAP-kinase mRNA. |
| 332 | 735647 | g1908564 | 233187.1 | 1681 | 0 | Human HSPC169 mRNA, complete cds. |
| 333 | 634252 | g1766738 | 474680.12 | 2104 | 0 | Human mRNA for Sec24 protein (Sec24B isoform). |
| 334 | 550700 | g1643510 | | | | |
| 335 | 597249 | g1714712 | | | | |
| 336 | 620453 | g1759420 | 726130.1 | 2139 | 0 | |
| 337 | 464803 | g1500830 | 260591.6 | 1825 | 0 | |
| 338 | 445075 | g4272043 | | | | |
| 339 | 472722 | g1513612 | | | | |
| 340 | 738162 | g1909378 | 332667.1 | 1871 | 6.00E−48 | Human mRNA; cDNA DKFZp56611247 (from clone DKFZp566I1247). |
| 341 | 642114 | g1808523 | 174021.1 | 1576 | 0 | |
| 342 | 426103 | g4199207 | | | | |
| 343 | 764212 | g1931980 | 1072489.2 | 1438 | 0 | |
| 344 | 523303 | g4623768 | 233046.2 | 1677 | 0 | Human mRNA for KIAA0382 gene, partial cds. |
| 345 | 736017 | g1908593 | 201056.2 | 1613 | 0 | Human cytokine-inducible SH2 protein 6 (CISH6) mRNA, complete |
| | | | | | | cds. |
| 346 | 803488 | g2201516 | | | | |
| 347 | 656375 | g1873357 | 201101.5 | 1615 | 0 | Human FK506-binding protein mRNA, complete cds. |
| 348 | 920701 | g2284349 | 330970.3 | 1853 | 4.00E−38 | Human reticulon gene family protein (RTN3) gene, exon 7 and |
| | | | | | | complete cds. |
| 349 | 492457 | g1643181 | | | | |
| 350 | 736232 | g1908766 | 334418.3 | 1877 | 0 | Human protein kinase mRNA, complete cds. |
| 351 | 424307 | g1428306 | 168836.3 | 1575 | 0 | Human Cak receptor kinase mRNA, complete cds. |
| 352 | 583186 | g1726467 | | | | |
| 353 | 418660 | g4275523 | 234636.5 | 1706 | 2.00E−93 | Human Hox5.4 gene fragment. |
| 354 | 493238 | g1643708 | 242309.3c | 1753 | 0 | Human mRNA for dihydropyrimidinase related protein-3, complete |
| | | | | | | cds. |
| 355 | 876058 | g2203273 | 244579.27c | 1765 | 0 | Human amyloid precursor protein homolog HSD-2 mRNA, complete |
| | | | | | | cds. |
| 356 | 617885 | g1756444 | 429727.5 | 2060 | 0 | Human fibrillin-2 mRNA, complete cds. |
| 357 | 419185 | g1542418 | | | | |
| 358 | 736526 | g4317916 | | | | |
| 359 | 737898 | g1917399 | | | | |
| 360 | 680964 | g1873049 | | | | |
| 361 | 481883 | g4057878 | | | | |
| 362 | 423599 | g4271828 | 1143741.1c | 1501 | 0 | |
| 363 | 636407 | g1765551 | 332404.6 | 1866 | 0 | |
| 364 | 481450 | g1554267 | | | | |
| 365 | 599075 | g1751902 | | | | |
| 366 | 573052 | g1676190 | | | | |
| 367 | 464165 | g1494519 | | | | |
| 368 | 622039 | g1767204 | 401619.5 | 2007 | 0 | Human zinc finger protein (MBLL) mRNA, complete cds. |
| 369 | 467107 | g1506427 | 250186.6 | 1792 | 0 | Human mRNA for procollagen alpha 2(V). |
| 370 | 807447 | g4320582 | 401619.11 | 2006 | 0 | Human zinc finger protein (MBLL) mRNA, complete cds. |
| 371 | 716523 | g1901594 | 902565.9c | 2203 | 0 | Human plasma membrane Ca2+ pumping ATPase mRNA, complete |
| | | | | | | cds. |
| 372 | 733746 | g1905104 | 234427.5 | 1704 | 0 | Human mRNA for GS3955, complete cds. |
| 373 | 817954 | g2192670 | | | | |
| 374 | 466730 | g1501087 | 1188552.2c | 1539 | 0 | Human protein phosphatase 2A beta subunit mRNA, complete |
| | | | | | | cds. |
| 375 | 763553 | g1929690 | 002588.4 | 1368 | 1.00E−40 | unknown orf, len: 393, CAI: 0.13 |
| 376 | 406947 | g1541090 | 241169.5 | 1750 | 0 | cyclophilin C [Human, kidney, mRNA, 883 nt]. |
| 377 | 622182 | g1767610 | | | | |
| 378 | 422477 | g1427462 | | | | |
| 379 | 949384 | g2288499 | | | | |
| 380 | 777321 | g1919540 | 401550.13 | 2002 | 5.00E−09 | Human mRNA; cDNA DKFZp434P1217 (from clone DKFZp434P1217); |
| | | | | | | partial cds. |
| 381 | 475285 | g4408840 | | | | |
| 382 | 493702 | g4273705 | | | | |
| 383 | 536577 | g1595710 | | | | |
| 384 | 466255 | g1504701 | | | | |
| 385 | 466178 | g1504783 | 199423.6 | 1603 | 1.00E−22 | copine III |
| 386 | 464415 | g1497650 | | | | |
| 387 | 423344 | g4271810 | 399595.1 | 1990 | 0 | Human full length insert cDNA clone YP83B01. |
| 388 | 637333 | g1768679 | | | | |
| 389 | 439638 | g1464684 | 390183.3c | 1980 | 0 | Human mRNA for embryonic myosin heavy chain. |
| 390 | 458712 | g1488806 | | | | |
| 391 | 672859 | g1853720 | 327521.13 | 1848 | 6.00E−30 | Human mRNA for histidyl-tRNA synthetase (HRS). |
| 392 | 467172 | g1506554 | 137252.5 | 1564 | 0 | Human parathyroid hormone/parathyroid hormone-related |
| | | | | | | peptide receptor mRNA, complete cds. |
| 393 | 400571 | g1387542 | | | | |
| 394 | 355517 | g1333025 | 399595.1 | 1990 | 0 | Human full length insert cDNA clone YP83B01. |
| 395 | 777529 | g1919306 | | | | |
| 396 | 533405 | g1654747 | 208832.8 | 1636 | 0 | Human clone 23771 mRNA sequence. |
| 397 | 680761 | g1873027 | | | | |
| 398 | 374296 | g1371733 | 476434.16 | 2118 | 0 | Human mRNA; cDNA DKFZp586B1417 (from clone DKFZp586B1417); |
| | | | | | | partial cds. |
| 399 | 774810 | g2039799 | 1071542.9c | 1422 | 0 | Human mRNA for KIAA1075 protein, partial cds. |
| 400 | 582063 | g1725958 | | | | |
| 401 | 643905 | g1800364 | 009165.8 | 1371 | 0 | Human ATP cassette binding transporter 1 (ABC1) mRNA, complete |
| | | | | | | cds. |
| 402 | 484125 | g1595433 | | | | |
| 403 | 332327 | g1282454 | | | | |
| 404 | 717466 | g2057400 | | | | |
| 405 | 421592 | g1427003 | 039966.3 | 1393 | 1.00E−91 | Human methyl-CpG binding protein 2 (MBD2) gene, exon 1. |
| 406 | 583632 | g1713380 | 245416.17 | 1774 | 0 | Human Mad4 homolog (Mad4) mRNA, complete cds. |
| 407 | 618535 | g1756599 | | | | |
| 408 | 761605 | g1910698 | 429406.3c | 2056 | 4.00E−95 | Human mRNA; cDNA DKFZp564C1940 (from clone DKFZp564C1940); |
| | | | | | | partial cds. |
| 409 | 693542 | g1863292 | 474928.4 | 2108 | 4.00E−50 | KIAA0970 protein |
| 410 | 751958 | g2049346 | | | | |
| 411 | 749073 | g1937165 | 475038.2 | 2110 | 0 | Human AA1 mRNA. |
| 412 | 484259 | g1619113 | | | | |
| 413 | 479920 | g1531457 | 1144093.1 | 1504 | 0 | Human putative tumor suppressor protein unspliced form (Fus-2) |
| | | | | | | mRNA, complete cds. |
| 414 | 891260 | g2248727 | | | | |
| 415 | 478904 | g1755298 | 474562.4 | 2100 | 0 | |
| 416 | 597342 | g1715393 | 812451.1 | 2167 | 0 | Human sodium/myo-inositol cotransporter (SLC5 A3) gene, |
| | | | | | | complete cds. |
| 417 | 439399 | g4315264 | 243308.3 | 1757 | 0 | |
| 418 | 638302 | g1795880 | | | | |
| 419 | 524119 | g1563106 | | | | |
| 420 | 864344 | g2180931 | | | | |
| 421 | 695508 | g1838335 | 1188730.2 | 1543 | 0 | Human selenoprotein N mRNA, partial cds. |
| 422 | 467480 | g1508946 | 899716.1 | 2199 | 2.00E−50 | Human (clone H4/H16) gamma-glutamic carboxylase mRNA, |
| | | | | | | complete cds. |
| 423 | 404526 | g4056811 | 1072584.3 | 1440 | 0 | Human translocon-associated protein gamma subunit mRNA, |
| | | | | | | complete cds. |
| 424 | 439732 | g1475306 | 407061.11 | 2021 | 0 | Human mRNA; cDNA DKFZp586N1922 (from clone DKFZp586N1922); |
| | | | | | | partial cds. |
| 425 | 403274 | g1539431 | | | | |
| 426 | 426493 | g1445785 | 243293.2 | 1756 | 4.00E−24 | Human ribosomal protein L37a (RPL37A) mRNA, complete cds. |
| 427 | 576974 | g1682062 | 1073084.13 | 1456 | 0 | Human transforming growth factor-beta type III receptor |
| | | | | | | (TGF-beta) mRNA, complete cds. |
| 428 | 720567 | g4765705 | | | | |
| 429 | 478156 | g1529273 | 1171690.1 | 1521 | 0 | Human AF-9 mRNA, complete cds. |
| 430 | 641871 | g1808211 | 328977.5 | 1849 | 0 | Human G-protein gamma-10 subunit mRNA, complete cds. |
| 431 | 622319 | g1758983 | 814644.23 | 2173 | 0 | Human protein tyrosine phosphatase (PTPase-alpha) mRNA. |
| 432 | 717910 | g1882293 | 256051.1 | | | |
| 433 | 476653 | g1530219 | 238040.3c | 1738 | 6.00E−22 | thrombomodulin precursor |
| 434 | 387151 | g1373933 | | | | |
| 435 | 762532 | g1909815 | | | | |
| 436 | 762728 | g1910000 | 113174.4c | 1494 | 2.00E−77 | Human Sox-4 mRNA. |
| 437 | 439803 | g1464508 | 765499.1c | 2157 | 0 | |
| 438 | 874540 | g2200305 | | | | |
| 439 | 831744 | g4616603 | | | | |
| 440 | 764518 | g1912735 | | | | |
| 441 | 436894 | g4199217 | 1094858.1 | 1488 | 0 | Human mRNA for B-FABP, complete cds. |
| 442 | 473770 | g1513824 | 762190.1 | 2151 | 0 | |
| 443 | 438506 | g4315201 | 347941.9 | 1928 | 0 | Human mRNA for KIAA0987 protein, partial cds. |
| 444 | 580505 | g1700169 | 816318.4 | 2181 | 0 | Human calcium-binding protein chp mRNA, complete cds. |
| 445 | 582571 | g1713535 | 233927.4 | 1697 | 0 | Human SM-11044 binding protein mRNA, partial cds. |
| 446 | 481880 | g4057877 | | | | |
| 447 | 478521 | g1529686 | 455057.6 | 2081 | 0 | L-arginine: glycine amidinotransferase [Human, kidney |
| | | | | | | carcinoma cells, mRNA, 2330 nt]. |
| 448 | 421537 | g4275663 | 081641.13c | 1403 | 0 | Human mRNA for KARP-1-binding protein 2 (KAB2), |
| | | | | | | complete cds. |
| 449 | 809031 | g2192179 | | | | |
| 450 | 390236 | g1385123 | | | | |
| 451 | 733517 | g1896508 | | | | |
| 452 | 597547 | g1713758 | 903441.9 | 2212 | 0 | Human P13-kinase associated p85 mRNA sequence. |
| 453 | 418495 | g1541869 | 025595.2 | 1384 | 0 | Human LAMB2 mRNA for beta2 laminin. |
| 454 | 331186 | g1287469 | 345146.9 | 1911 | 0 | Human caveolin 1 (CAV1) gene, exon 3 and partial cds. |
| 455 | 694133 | g1863090 | 1072976.4 | 1445 | 0 | Human ezrin-radixin-moesin binding phosphoprotein-50 mRNA, |
| | | | | | | complete cds. |
| 456 | 776562 | g1918519 | | | | |
| 457 | 619201 | g1756813 | 423565.22 | 2049 | 0 | Human WSB-1 isoform mRNA, complete cds. |
| 458 | 418453 | g4275517 | 197929.1 | 1594 | 0 | Human full length insert cDNA clone ZD41E06. |
| 459 | 440843 | g1464807 | 200009.2 | 1609 | 0 | |
| 460 | 457264 | g1487595 | 012942.16 | 1375 | 0 | Human skeletal muscle LIM-protein SLIM3 mRNA, partial cds. |
| 461 | 332687 | g1282686 | | | | |
| 462 | 598305 | g1740058 | 238120.1 | 1740 | 0 | Human mRNA; cDNA DKFZp586N0721 (from clone DKFZp586N0721). |
| 463 | 466905 | g1501227 | 336968.1 | 1889 | 0.0006 | ORF-3 protein |
| 464 | 574888 | g1676029 | 397963.12 | 1984 | 0 | Human arginine methyltransferase mRNA, complete cds. |
| 465 | 679641 | g1873545 | 244322.33 | 1763 | 0 | Human mRNA for KIAA0251 gene, partial cds. |
| 466 | 734062 | g1910437 | 241227.8 | 1751 | 0 | Human unknown mRNA. |
| 467 | 576216 | g1677377 | 1006060.1 | 1408 | 0 | |
| 468 | 620333 | g1759076 | 474360.6 | 2096 | 0 | Human Smad1 mRNA, complete cds. |
| 469 | 441276 | g1472901 | | | | |
| 470 | 476019 | g1755257 | 074456.1 | 1401 | 0.001 | salivary proline-rich protein precursor |
| 471 | 354601 | g1330439 | 1072389.2 | 1433 | 0 | Human ELP-1 mRNA sequence. |
| 472 | 474107 | g4623821 | | | | |
| 473 | 420709 | g1542184 | 404716.8 | 2018 | 0 | Human desmoplakin I (DPI) mRNA, complete cds. |
| 474 | 621389 | g1760003 | | | | |
| 475 | 717316 | g2057838 | 1071390.7 | 1416 | 1.00E−11 | unknown protein |
| 476 | 493109 | g1642864 | 902527.2 | 2202 | 0 | Human WDR1 protein mRNA, complete cds. |
| 477 | 403384 | g1539501 | 233660.5 | 1689 | 3.00E−32 | ubiquitin-specific protease |
| 478 | 483024 | g1554701 | 075492.97 | 1402 | 0 | Human alpha gene sequence. |
| 479 | 572889 | g1662698 | 1073021.18 | 1451 | 0 | Human mRNA for BS69 protein. |
| 480 | 733557 | g1895904 | 814642.2 | 2172 | 0 | Human partial cDNA sequence, clone bs86;. |
| 481 | 478265 | g1529416 | | | | |
| 482 | 423915 | g1542742 | 199975.3 | 1606 | 0 | Human mRNA for KIAA1249 protein, partial cds. |
| 483 | 695447 | g1838303 | | | | |
| 484 | 640875 | g1807751 | 1073172.1 | 1460 | 6.00E−18 | Human mRNA for KIAA0280 gene, partial cds. |
| 485 | 480526 | g4061314 | | | | |
| 486 | 696489 | g1863752 | | | | |
| 487 | 639911 | g1793063 | | | | |
| 488 | 874154 | g2200262 | 438393.1 | 2072 | 0 | |
| 489 | 777640 | g1919644 | 373272.16 | 1972 | 0 | Human mRNA for serum protein. |
| 490 | 888624 | g2233824 | 430153.7 | 2064 | 0 | Human mRNA for 3′,5′-cyclic GMP phosphodiesterase, |
| | | | | | | complete cds. |
| 491 | 762542 | g1909872 | 287029.4c | 1839 | 0 | Human mRNA for PIG-F (phosphatidyl-inositol-glycan class F), |
| | | | | | | complete cds. |
| 492 | 875543 | g2203333 | 440122.23 | 2073 | 0 | Human archvillin (SVIL) mRNA, complete cds. |
| 493 | 439959 | g4315296 | | | | |
| 494 | 749144 | g1937196 | | | | |
| 495 | 922965 | g2249219 | 245992.3 | 1780 | 0 | Human CGI-123 protein mRNA, complete cds. |
| 496 | 721040 | g1904106 | | | | |
| 497 | 480149 | g1531416 | | | | |
| 498 | 596754 | g1700680 | | | | |
| 499 | 920211 | g2235263 | 233017.4 | 1676 | 0 | Human full length insert cDNA clone YB21H04. |
| 500 | 457289 | g1487605 | 196915.1 | 1587 | 0 | Human mRNA; cDNA DKFZp586H0924 (from clone DKFZp586H0924). |
| 501 | 355990 | g1337506 | 1073075.15 | 1454 | 0 | Human chondroitin sulfate proteoglycan versican V0 splice- |
| | | | | | | variant precursor peptide mRNA, complete cds. |
| 502 | 354506 | g1330567 | 244823.1 | 1769 | 0 | Human mRNA for protein, kinase Dyrk2. |
| 503 | 424979 | g1435853 | | | | |
| 504 | 697506 | g1888261 | | | | |
| 505 | 419788 | g4276191 | | | | |
| 506 | 402614 | g1389111 | 1074263.1 | 1463 | 2.00E−69 | precursor cystatin C C-terminal fragment (128 AA) (1 is 2nd |
| | | | | | | base in codon) |
| 507 | 459254 | g1497391 | 306467.1 | 1845 | 0 | Human mRNA for KIAA1048 protein, complete cds. |
| 508 | 672351 | g1853873 | | | | |
| 509 | 466715 | g1501032 | 029170.7 | 1387 | 2.00E−36 | Human mRNA for KIAA0964 protein, complete cds. |
| 510 | 735186 | g1896159 | 1188593.1c | 1540 | 0 | Human CGI-63 protein mRNA, complete cds. |
| 511 | 314394 | g1286865 | 110671.1 | 1491 | 0 | |
| 512 | 748241 | g1914982 | 234157.2 | 1701 | 0 | Human chromosome 16 open reading frame 5 (C16orf5) mRNA, |
| | | | | | | complete cds. |
| 513 | 421973 | g4275694 | | | | |
| 514 | 621316 | g1759898 | 336239.5 | 1884 | 0 | Human cobalt inducable gene-18. |
| 515 | 404339 | g1540164 | | | | |
| 516 | 764361 | g1917253 | | | | |
| 517 | 482328 | g1554719 | 255931.2 | 1808 | 0 | Human mRNA; cDNA DKFZp564O123 (from clone DKFZp564O123); |
| | | | | | | complete cds. |
| 518 | 775912 | g1919033 | 236455.25 | 1729 | 0 | Human mRNA; cDNA DKFZp434P106 (from clone DKFZp434P106). |
| 519 | 571643 | g1660314 | 480992.10c | 2129 | 0 | Human 9-cis-retinol specific dehydrogenase mRNA, |
| | | | | | | complete cds. |
| 520 | 678740 | g1861388 | 425074.21 | 2051 | 0 | Human rac protein kinase alpha mRNA, complete cds. |
| 521 | 618644 | g1748887 | 410523.5 | 2031 | 0 | Human guanine nucleotide exchange factor mRNA, |
| | | | | | | complete cds. |
| 522 | 763021 | g1910069 | | | | |
| 523 | 641451 | g1796607 | 476434.22 | 2119 | 0 | Human mRNA; cDNA DKFZp586B1417 (from clone DKFZp586B1417); |
| | | | | | | partial cds. |
| 524 | 314127 | g1284229 | 401617.5 | 2005 | 1.00E−81 | Human mRNA; cDNA DKFZp586H051 (from clone DKFZp586H051). |
| 525 | 596968 | g1701096 | | | | |
| 526 | 803358 | g2201244 | 234748.5 | 1708 | 3.00E−18 | parathion hydrolase (phosphotriesterase)-related protein |
| 527 | 390390 | g1380956 | | | | |
| 528 | 875444 | g2203197 | 349727.7 | 1935 | 0 | Human G-protein-coupled receptor kinase (GRK5) mRNA, |
| | | | | | | complete cds. |
| 529 | 820354 | g4320896 | 018683.1 | 1378 | 0 | Human mRNA for Slit-3 protein, complete cds. |
| 530 | 736616 | g1909000 | 902725.1 | 2204 | 0 | Human mRNA for laminin alpha 4 protein. |
| 531 | 876166 | g2204008 | 235180.8 | 1715 | 2.00E−73 | Human full length insert cDNA clone ZD76G10. |
| 532 | 904738 | g2262509 | | | | |
| 533 | 775150 | g2039860 | 340580.28 | 1897 | 0 | Human genomic DNA of 21q22.2 Down Syndrome region, |
| | | | | | | segment 2/13. |
| 534 | 671660 | g1872625 | 398839.6 | 1985 | 0 | Human chromosome X region from filamin (FLN) gene to |
| | | | | | | glucose-6-phosphate dehydrogenase (G6PD) gene, |
| | | | | | | complete cds's. |
| 535 | 373793 | g1372276 | 245886.11 | 1778 | 0 | Human mRNA for calponin, complete cds. |
| 536 | 596200 | g1681701 | | | | |
| 537 | 652498 | g1826320 | | | | |
| 538 | 658233 | g1793900 | | | | |
| 539 | 442842 | g1476026 | 331792.1 | 1862 | 0 | Human mRNA; cDNA DKFZp564G2463 (from clone |
| | | | | | | DKFZp564G2463). |
| 540 | 774877 | g2039712 | | | | |
| 541 | 421530 | g1427184 | | | | |
| 542 | 419553 | g1542538 | | | | |
| 543 | 386236 | g1372731 | | | | |
| 544 | 418531 | g1541886 | | | | |
| 545 | 439010 | g1464219 | | | | |
| 546 | 643384 | g1772257 | | | | |
| 547 | 464245 | g1504318 | | | | |
| 548 | 718110 | g1902551 | | | | |
| 549 | 902264 | g2256372 | 410012.16 | 2030 | 0 | Human HSPC292 mRNA, partial cds. |
| 550 | 718293 | g1895306 | 004943.1 | 1370 | 3.00E−48 | erythropoietin receptor precursor |
| 551 | 523088 | g1565032 | 235147.76 | 1712 | 0 | Human calumein (Calu) mRNA, complete cds. |
| 552 | 423219 | g4275742 | 331042.1 | 1855 | 0 | Human mRNA for KIAA1094 protein, complete cds. |
| 553 | 762782 | g1910032 | 1080543.1 | 1473 | 0 | |
| 554 | 572156 | g1654977 | 478176.6 | 2120 | 0 | Human T245 protein (T245) mRNA, complete cds. |
| 555 | 763989 | g1929706 | | | | |
| 556 | 463860 | g1494600 | | | | |
| 557 | 670375 | g1840508 | | | | |
| 558 | 406689 | g4257398 | | | | |
| 559 | 466155 | g1504779 | | | | |
| 560 | 622792 | g1758855 | | | | |
| 561 | 597729 | g4060433 | 399489.1 | 1989 | 0 | Human tactile protein mRNA, complete cds. |
| 562 | 472407 | g1509530 | 410660.21 | 2033 | 0 | Human enigma gene, complete cds. |
| 563 | 765073 | g1912816 | | | | |
| 564 | 922216 | g2250492 | 137946.2 | 1565 | 6.00E−08 | ORF MSV242 putative inhibitor of apoptosis protein (IAP), |
| | | | | | | similar to Autographa californica NPV GB:D36828 |
| 565 | 677412 | g1811582 | 235197.9c | 1716 | 0 | Human protein-tyrosine phosphatase (PTPase MEG2) mRNA, |
| | | | | | | complete cds. |
| 566 | 722451 | g1888830 | | | | |
| 567 | 846131 | g2076192 | 034500.3 | 1390 | 0 | |
| 568 | 762142 | g1917792 | 349617.2 | 1933 | 0 | Human DNA-binding protein mRNA, 3′ end. |
| 569 | 934635 | g2282831 | | | | |
| 570 | 332091 | g1282177 | | | | |
| 571 | 329780 | g1286926 | 331908.5 | 1863 | 0 | Human mRNA for p cadherin. |
| 572 | 574476 | g1684321 | | | | |
| 573 | 476342 | g1530321 | 1088480.1 | 1484 | 0 | Human mRNA; cDNA DKFZp564A2416 (from clone DKFZp564A2416). |
| 574 | 482943 | g4273558 | 233550.1 | 1687 | 0 | odd-skipped related 1 protein |
| 575 | 680103 | g4306694 | | | | |
| 576 | 574134 | g1676531 | | | | |
| 577 | 334410 | g1293946 | | | | |
| 578 | 872869 | g2192572 | 399101.6c | 1987 | 0 | Human mRNA for S100 alpha protein. |
| 579 | 749776 | g1937662 | 1189233.1c | 1547 | 0 | Human mRNA for KIAA0623 protein, complete cds. |
| 580 | 749282 | g1937085 | | | | |
| 581 | 820498 | g2102189 | | | | |
| 582 | 317778 | g1287080 | | | | |
| 583 | 439784 | g1464549 | | | | |
| 584 | 426076 | g1436861 | | | | |
| 585 | 336726 | g4258568 | 411151.3c | 2037 | 0 | Human mRNA for ZASP protein, partial. |
| 586 | 479750 | g1531290 | | | | |
| 587 | 353229 | g1325860 | | | | |
| 588 | 777101 | g1918994 | 349746.6 | 1937 | 0 | Human MADS/MEF2-family transcription factor (MEF2C) mRNA, |
| | | | | | | complete cds. |
| 589 | 330370 | g4257102 | | | | |
| 590 | 656701 | g1864627 | | | | |
| 591 | 622653 | g1758772 | 292613.13 | 1842 | 5.00E−35 | Human CGI-72 protein mRNA, complete cds. |
| 592 | 425348 | g1435908 | | | | |
| 593 | 423177 | g1427142 | 814118.1 | 2170 | 0 | |
| 594 | 419633 | g1538799 | 345309.9 | 1914 | 0 | Human mRNA for endothelin-converting enzyme, complete cds. |
| 595 | 635569 | g1743958 | 481144.8 | 2131 | 0 | Human PAK2 mRNA, complete cds. |
| 596 | 493460 | g1630169 | | | | |
| 597 | 777306 | g1919658 | | | | |
| 598 | 761298 | g2041294 | | | | |
| 599 | 479846 | g1531151 | | | | |
| 600 | 595978 | g1699549 | | | | |
| 601 | 850389 | g2164914 | | | | |
| 602 | 888687 | g2233780 | 333812.6 | 1875 | 0 | |
| 603 | 478848 | g4057832 | | | | |
| 604 | 477665 | g4276659 | 353740.3 | 1957 | 0 | Human PHD-finger protein (GRC5) mRNA, complete cds. |
| 605 | 421653 | g1428844 | 1189864.1 | 1555 | 1.00E−10 | agrin |
| 606 | 622359 | g4766055 | | | | |
| 607 | 637536 | g1768408 | | | | |
| 608 | 425653 | g1436197 | 813260.1 | 2168 | 0 | Human heart chymase mRNA, complete cds. |
| 609 | 641660 | g1808314 | | | | |
| 610 | 477177 | g1527804 | 039466.2 | 1391 | 0 | Human RNB6 (RNB6) mRNA, complete cds. |
| 611 | 419846 | g1538910 | | | | |
| 612 | 385072 | g1369680 | 350447.13c | 1947 | 1.00E−85 | Human src-like kinase (slk) mRNA, complete cds. |
| 613 | 935626 | g2292157 | | | | |
| 614 | 483763 | g4057914 | | | | |
| 615 | 476321 | g1530261 | 211736.9 | 1640 | 0 | Human semaphorin III family homolog mRNA, complete cds. |
| 616 | 748112 | g1897006 | 093687.7 | 1405 | 0 | Human uncoupling protein-2 (UCP2) gene, nuclear gene |
| | | | | | | encoding mitochondrial protein, complete cds. |
| 617 | 641523 | g1796629 | 429857.16 | 2062 | 0 | Human zinc ringer transcriptional regulator (GOS24) gene, |
| | | | | | | complete cds. |
| 618 | 422094 | g1427202 | 250793.3 | 1793 | 0 | growth suppressor 1L |
| 619 | 637934 | g1776582 | | | | |
| 620 | 468472 | g1475255 | 474411.49 | 2097 | 0 | Human syntaxin 7 mRNA, complete cds. |
| 621 | 641806 | g1808488 | | | | |
| 622 | 621352 | g1758571 | | | | |
| 623 | 775722 | g1918497 | | | | |
| 624 | 638593 | g4522215 | 1019119.1c | 1412 | 0 | |
| 625 | 406222 | g1540639 | | | | |
| 626 | 583823 | g1715789 | 1190143.1c | 1557 | 3.00E−17 | Similar to Rat trg gene product; coded for by C. elegans cDNA |
| | | | | | | yk31e7.5; coded for by C. elegans cDNA yk40d6.5; coded for by |
| | | | | | | C. elegans cDNA yk31e7.3; coded for by C. elegans cDNA yk40d6.3; |
| | | | | | | coded for by C. elegans cDNA yk149g5.3; coded for by C> |
| 627 | 571503 | g1660682 | | | | |
| 628 | 681670 | g1862017 | | | | |
| 629 | 721482 | g1903673 | | | | |
| 630 | 776770 | g1913500 | 1079214.1 | 1471 | 1.00E−30 | NBAT |
| 631 | 423210 | g1427173 | | | | |
| 632 | 598956 | g1746848 | 900028.1 | 2200 | 6.00E−45 | Human glycophorin C (PAS-2) mRNA, complete cds. |
| 633 | 660997 | g1888209 | | | | |
| 634 | 751729 | g2049156 | 174273.1 | 1579 | 0 | |
| 635 | 535228 | g1629910 | 474891.5 | 2107 | 0 | Human dynactin p62 subunit mRNA, complete cds. |
| 636 | 464134 | g1494446 | 435298.1 | 2067 | 0 | |
| 637 | 737934 | g1909346 | 406531.1 | 2020 | 0 | Human bone morphogenetic protein 5 (BMP-5) mRNA, complete cds. |
| 638 | 697509 | g1888265 | 899062.1 | 2194 | 1.00E−99 | Human OS-4 protein (OS-4) mRNA, complete cds. |
| 639 | 481375 | g1554177 | | | | |
| 640 | 420487 | g1542044 | 410561.3 | 2032 | 0 | Human voltage-gated calcium channel beta subunit mRNA, |
| | | | | | | complete cds. |
| 641 | 574756 | g1682468 | 116417.8 | 1510 | 0 | Human RP58 gene, complete cds. |
| 642 | 621226 | g1759846 | 903485.2 | 2214 | 0 | Human receptor-associated tyrosine kinase (JAK2) mRNA, |
| | | | | | | complete cds. |
| 643 | 480431 | g1551147 | | | | |
| 644 | 427349 | g4271980 | | | | |
| 645 | 699290 | g1876308 | 411148.2 | 2036 | 0 | Human mRNA for transducin (beta) like 1 protein. |
| 646 | 670886 | g1841284 | 898101.24 | 2189 | 0 | Human RHOA proto-oncogene multi-drug-resistance protein mRNA, |
| | | | | | | 3′ end. |
| 647 | 385725 | g1371890 | 431802.7 | 2066 | 0 | |
| 648 | 818435 | g2139651 | 010652.1 | 1372 | 0 | |
| 649 | 717056 | g1904436 | | | | |
| 650 | 385435 | g1371803 | 400575.4 | 1995 | 0 | Human mRNA for KIAA0630 protein, partial cds. |
| 651 | 445817 | g4057739 | 411135.6 | 2035 | 0 | Human fra-2 mRNA. |
| 652 | 949003 | g2292273 | | | | |
| 653 | 478711 | g1531055 | | | | |
| 654 | 387594 | g1375763 | | | | |
| 655 | 746888 | g4616861 | | | | |
| 656 | 474184 | g1514146 | | | | |
| 657 | 423531 | g4271823 | 417948.2 | 2046 | 0 | |
| 658 | 463575 | g1500768 | 1072534.1 | 1439 | 0 | Human peroxisomal D3,D2-enoyl-CoA isomerase (PECI) mRNA, |
| | | | | | | complete cds. |
| 659 | 948588 | g2305796 | 198215.1 | 1598 | 0 | |
| 660 | 464866 | g1497659 | | | | |
| 661 | 597832 | g1739402 | 357143.4 | 1958 | 0 | Human apoptosis-related RNA binding protein (NAPOR-3) mRNA, |
| | | | | | | complete cds. |
| 662 | 876381 | g2203656 | 424444.21 | 2050 | 0 | Human LSFR1 gene, sixth to eigth of eight identified exons. |
| 663 | 905429 | g2271843 | | | | |
| 664 | 401764 | g1388816 | 401705.4 | 2008 | 0 | Human frequenin mRNA, complete cds. |
| 665 | 347921 | g1309848 | 903876.6 | 2215 | 0 | Human protein-tyrosine kinase (JAK1) mRNA, complete cds. |
| 666 | 736724 | g1909293 | 115947.12 | 1507 | 0 | Human cAMP-dependent protein kinase regulatory subunit |
| | | | | | | RI-beta mRNA, 3′ end. |
| 667 | 618375 | g1756304 | | | | |
| 668 | 402843 | g1391292 | | | | |
| 669 | 751577 | g2042938 | 474588.15 | 2101 | 0 | Human polyadenylate binding protein (TIA-1) mRNA, |
| | | | | | | complete cds. |
| 670 | 677054 | g1811857 | 232986.4c | 1675 | 0 | Human mRNA; cDNA DKFZp434D156 (from clone DKFZp434D156); |
| | | | | | | partial cds. |
| 671 | 695231 | g1838312 | 481960.8 | 2136 | 0 | Human liprin-beta1 mRNA, complete cds. |
| 672 | 576022 | g1676639 | 334355.14 | 1876 | 0 | Human mRNA; cDNA DKFZp434E171 (ftom clone DKFZp434E171); |
| | | | | | | partial cds. |
| 673 | 536574 | g1595625 | 345283.14 | 1913 | 0 | Human regulator of G-protein signaling RGS12 (RGS) mRNA, |
| | | | | | | complete cds. |
| 674 | 888544 | g2233811 | 236836.7 | 1732 | 0 | Human CGI-38 protein mRNA, complete cds. |
| 675 | 576003 | g4300335 | | | | |
| 676 | 642330 | g1768894 | 233910.3 | 1696 | 0 | Human mRNA for KIAA1008 protein, complete cds. |
| 677 | 334138 | g1537441 | 350234.5 | 1944 | 2.00E−55 | ORF |
| 678 | 751348 | g1937073 | 243989.7 | 1760 | 0 | Human Mago homolog mRNA, complete cds. |
| 679 | 479576 | g1528656 | 135490.11c | 1563 | 0 | Human mRNA for KIAA1007 protein, partial cds. |
| 680 | 351163 | g1324773 | | | | |
| 681 | 403660 | g1539702 | 401575.1 | | | |
| 682 | 671530 | g1865589 | 1175282.1 | 1524 | 0 | Human mRNA for basic transcription factor 2, 34 kD subunit. |
| 683 | 532186 | g1566657 | 1072468.2 | 1437 | 0 | Human, NAD(P)H: menadione oxidoreductase mRNA, complete cds. |
| 684 | 934425 | g4606070 | 239750.2 | 1748 | 0 | |
| 685 | 476367 | g1530332 | | | | |
| 686 | 736297 | g1908935 | 757594.5 | 2146 | 2.00E−92 | Human D38 mRNA. |
| 687 | 425523 | g4271900 | 202281.2 | 1627 | 0 | SPARC-related protein |
| 688 | 636193 | g1766844 | 197486.9c | 1590 | 0 | Human mRNA for VRK2, complete cds. |
| 689 | 833508 | g2192120 | 344543.16c | 1906 | 0 | Human mRNA for 26S proteasome subunit p44.5, complete cds. |
| 690 | 656956 | g1864871 | 757538.7 | 2143 | 0 | Human cell cycle progression 2 protein (CPR2) mRNA, |
| | | | | | | complete cds. |
| 691 | 733856 | g1904900 | 233958.2 | 1698 | 0 | Human calpain-like protease (htra-3) mRNA, complete cds. |
| 692 | 820430 | g2102146 | 428087.2 | 2055 | 3.00E−54 | Human GA17 protein mRNA, complete cds. |
| 693 | 616975 | g1744427 | 1081629.1 | 1474 | 0 | Human mRNA for DRM protein. |
| 694 | 418563 | g1428966 | 474965.3 | 2109 | 0 | 26 S protease subunit 5b = 50 kda subunit [Human, |
| | | | | | | HeLa cells, mRNA Partial, 2253 nt]. |
| 695 | 640026 | g1793064 | 421854.2 | 2047 | 0 | Human centrosomal protein kendrin mRNA, complete cds. |
| 696 | 948662 | g2291590 | 135490.11c | 1563 | 0 | Human mRNA for KIAA1007 protein, partial cds. |
| 697 | 894275 | g2256859 | 1072970.5 | 1444 | 3.00E−10 | mitochondrial solute carrier |
| 698 | 737800 | g1917863 | 344200.3 | 1903 | 0 | Human zinc ringer DNA binding protein 99 (ZNF281) mRNA, |
| | | | | | | complete cds. |
| 699 | 833284 | g2191986 | | | | |
| 700 | 791460 | g2075608 | | | | |
| 701 | 671801 | g1872777 | 435550.1 | 2068 | 6.00E−90 | Human full length insert cDNA clone YZ83B08. |
| 702 | 889470 | g2233541 | 331585.8 | 1860 | 6.00E−70 | elongation factor G |
| 703 | 472805 | g4059293 | 903095.1 | 2207 | 7.00E−65 | Human chromodomain-helicase-DNA-binding protein mRNA, |
| | | | | | | complete cds. |
| 704 | 597797 | g1740027 | 138082.1 | 1566 | 0 | |
| 705 | 425442 | g4271893 | 350396.1 | 1945 | 6.00E−65 | Human CpG island DNA genomic Msel fragment, clone 28b4, |
| | | | | | | forward read cpg28b4.ft1a. |
| 706 | 438286 | g1464386 | 404616.2 | 2016 | 0 | pyridoxine 5′-phosphate oxidase |
| 707 | 424618 | g4271855 | | | | |
| 708 | 808777 | g2193625 | 404508.14 | 2014 | 0 | Human mRNA for KIAA0896 protein, partial cds. |
| 709 | 389279 | g1380076 | 161166.2 | 1574 | 0 | Human RNaseP protein p30 (RPP30) mRNA, complete cds. |
| 710 | 425081 | g1435802 | 475291.7 | 2113 | 1.00E−98 | Human RNA polymerase I 16 kDa subunit mRNA, complete cds. |
| 711 | 749766 | g4766354 | | | | |
| 712 | 791058 | g2075289 | 1185880.1c | 1532 | 0 | Human mRNA for hSNF2H, complete cds. |
| 713 | 699378 | g4315392 | | | | |
| 714 | 579733 | g1671962 | | | | |
| 715 | 640062 | g1792969 | 899418.2 | 2198 | 0 | Human tumor suppressing STF cDNA 1 (TSSC1) mRNA, |
| | | | | | | complete cds. |
| 716 | 761554 | g1910664 | 238950.7 | 1745 | 0 | Human mRNA; cDNA DKFZp566C0424 (from clone DKFZp566C0424); |
| | | | | | | partial cds. |
| 717 | 618630 | g1756110 | 476034.4 | 2116 | 2.00E−94 | Human znf6 mRNA for zinc finger transcription factor. |
| 718 | 390118 | g1380489 | 394963.6 | 1982 | 0 | Human mRNA for KIAA0011 gene, complete cds. |
| 719 | 735147 | g1895925 | | | | |
| 720 | 640178 | g4804880 | | | | |
| 721 | 534015 | g1594589 | | | | |
| 722 | 423081 | g1427956 | 191737.1c | 1585 | 1.00E−13 | R04E5.2 gene product |
| 723 | 400308 | g1387448 | 425487.5 | 2052 | 0 | Human mRNA for KIAA1080 protein, partial cds. |
| 724 | 751619 | g2049091 | | | | |
| 725 | 780092 | g2074942 | 350100.35 | 1940 | 0 | Human set gene, complete cds. |
| 726 | 316237 | g1283201 | 1094181.3c | 1486 | 0 | RDP = renal dipeptidase [Human, kidney, Genomic, |
| | | | | | | 3570 nt, segment 2 of 2]. |
| 727 | 476022 | g1755255 | | | | |
| 728 | 597201 | g1714701 | | | | |
| 729 | 352795 | g4258974 | | | | |
| 730 | 465555 | g1504185 | | | | |
| 731 | 807462 | g2071508 | 257179.6 | 1818 | 0 | Human full length insert cDNA clone YU03D12. |
| 732 | 762210 | g1917812 | 243812.2 | 1759 | 0 | Human stress-activated protein kinase 4 mRNA, |
| | | | | | | complete cds. |
| 733 | 351221 | g4199085 | 336147.1 | 1883 | 0 | Human glycerol-3-phosphate dehydrogenase mRNA, nuclear |
| | | | | | | gene encoding mitochondrial protein, complete cds. |
| 734 | 472664 | g1513402 | 220839.3 | 1645 | 6.00E−89 | KIAA1078 protein |
| 735 | 400408 | g4257438 | 199099.3 | 1602 | 2.00E−29 | R28379_3 |
| 736 | 350881 | g1325311 | 1084837.1 | 1477 | 3.00E−25 | similar to carrier protein C2; cDNA EST EMBL:Z14780 comes |
| | | | | | | from this gene; cDNA EST EMBL:Z14920 comes from this gene; |
| | | | | | | cDNA EST EMBL:M89112 comes from this gene; cDNA EST |
| | | | | | | yk458c8.5 comes from this gene; cDNA EST yk458c8.3 comes |
| | | | | | | from this gene; c> |
| 737 | 424559 | g1428189 | 235169.1 | 1714 | 5.00E−93 | Human HSPC316 mRNA, partial cds. |
| 738 | 573595 | g1662629 | | | | |
| 739 | 699377 | g1876278 | 197003.1 | 1588 | 0 | |
| 740 | 386234 | g1372730 | 257576.34 | 1822 | 0 | Human mRNA for novel heterogeneous nuclear RNP protein, |
| | | | | | | L protein. |
| 741 | 576991 | g1681607 | 201417.15 | 1618 | 0 | Human U4/U6 small nuclear ribonucleoprotein hPrp3 mRNA, |
| | | | | | | complete cds. |
| 742 | 317597 | g1285746 | 350777.7 | 1949 | 0 | |
| 743 | 577676 | g4061823 | 407724.1 | 2025 | 0 | Human mRNA for GTPase activating protein ID-GAP, |
| | | | | | | complete cds. |
| 744 | 698109 | g1863171 | 253617.12 | 1801 | 0 | Human gene for mitochondrial ATP synthase c |
| | | | | | | subunit (P2 form). |
| 745 | 437685 | g1463796 | | | | |
| 746 | 762306 | g1917901 | | | | |
| 747 | 354969 | g1330700 | | | | |
| 748 | 332285 | g1282707 | | | | |
| 749 | 464392 | g4058645 | | | | |
| 750 | 406706 | g1394621 | 024513.4 | 1383 | 2.00E−11 | Ydr449cp; CAI: 0.18 |
| 751 | 804212 | g2202679 | 331647.2 | 1861 | 0 | Human mRNA; cDNA DKFZp434G0972 (from clone DKFZp434G0972). |
| 752 | 581115 | g1702222 | | | | |
| 753 | 315536 | g1284151 | | | | |
| 754 | 639975 | g1793232 | | | | |
| 755 | 580166 | g1699962 | | | | |
| 756 | 482432 | g4603968 | | | | |
| 757 | 697208 | g1841066 | | | | |
| 758 | 478334 | g4057824 | 232344.1c | 1670 | 0 | Human lamin B2 (LAMB2) gene and ppv1 gene sequence. |
| 759 | 403381 | g1539508 | 1077535.1 | 1466 | 4.00E−28 | helicase |
| 760 | 779373 | g2074977 | | | | |
| 761 | 441227 | g1472886 | 366108.8 | 1967 | 3.00E−43 | Human genomic DNA fragment (clone NL2A230R). |
| 762 | 746908 | g1915033 | | | | |
| 763 | 775188 | g2040160 | | | | |
| 764 | 351869 | g1324943 | | | | |
| 765 | 439640 | g1464685 | 903951.15 | 2217 | 0 | ArgRS = arginyl-tRNA synthetase [Human, |
| | | | | | | ataxia-telangiectasia patients, EBV-lymphoblastoid cells, |
| | | | | | | mRNA, 2120 nt]. |
| 766 | 620209 | g1759154 | 1189704.1 | 1551 | 0 | |
| 767 | 318957 | g1316157 | 245970.4 | 1779 | 0 | Human alpha 1-syntrophin (SNT A1) mRNA, complete cds. |
| 768 | 468823 | g1509030 | 230893.12 | 1665 | 0 | Human DNA polymerase delta small subunit mRNA, |
| | | | | | | complete cds. |
| 769 | 618926 | g1756624 | | | | |
| 770 | 353445 | g1325643 | 474244.9 | 2093 | 0 | Human cleavage and polyadenylation specificity factor mRNA, |
| | | | | | | complete cds. |
| 771 | 920442 | g2284501 | 346108.11 | 1915 | 2.00E−96 | Human BTK region clone 2f10-rpi mRNA. |
| 772 | 443441 | g1475077 | 333506.1 | 1874 | 0 | Human nuclear FMRP interacting protein 1 (NUFIP1) mRNA, |
| | | | | | | complete cds. |
| 773 | 403946 | g1539872 | | | | |
| 774 | 442662 | g1476176 | 1086324.1 | 1483 | 0 | Human mRNA for elongation factor-1-beta. |
| 775 | 484299 | g1619178 | 282761.6c | 1835 | 0 | Human end mRNA for endoglin. |
| 776 | 639902 | g1792952 | 1188855.2 | 1544 | 6.00E−08 | eyelid |
| 111 | 479201 | g4273404 | | | | |
| 778 | 961056 | g4401577 | | | | |
| 779 | 961282 | g2308846 | 233889.3 | 1695 | 0 | Human ckshs1 mRNA for Cks1 protein homologue. |
| 780 | 373505 | g1365775 | | | | |
| 781 | 894169 | g2256632 | | | | |
| 782 | 419193 | g1542420 | 474597.1 | 2103 | 0 | Human nuclear factor p97 (NTF97) gene, complete cds. |
| 783 | 720904 | g4765743 | | | | |
| 784 | 671169 | g1842625 | 233723.2 | 1691 | 0 | |
| 785 | 920335 | g4405504 | | | | |
| 786 | 638129 | g4296087 | 399465.4 | 1988 | 0 | Human mRNA for KIAA0136 gene, partial cds, |
| 787 | 535949 | g4060161 | 205575.15 | 1633 | 0 | Human activation of Sentrin/SUMO protein AOS1 (AOS1) mRNA, |
| | | | | | | complete cds. |
| 788 | 336325 | g1537507 | 255781.16 | 1805 | 0 | Human mRNA for connective tissue growth factor. |
| 789 | 577766 | g1701429 | 898733.2 | 2191 | 2.00E−32 | Prodos protein |
| 790 | 419886 | g4199169 | | | | |
| 791 | 750205 | g2049494 | 760717.1 | 2149 | 2.00E−76 | Human YEAF1 mRNA for YY1 and E4TF1 associated factor 1, |
| | | | | | | complete cds. |
| 792 | 483584 | g1555700 | 475486.1 | 2114 | 9.00E−82 | Human cyclophilin isoform (hCyP3) mRNA, complete cds. |
| 793 | 523965 | g1566011 | 233592.5 | 1688 | 0 | Human mRNA for A-kinase anchoring protein AKAP95. |
| 794 | 439665 | g1475372 | 413452.77 | 2043 | 0 | |
| 795 | 642201 | g4304497 | | | | |
| 796 | 633284 | g1759730 | 1188225.1 | 1537 | 0 | |
| 797 | 692936 | g1862500 | 816001.1 | 2179 | 0 | Human mRNA for transcription factor BTF 3. |
| 798 | 572463 | g1662557 | | | | |
| 799 | 581799 | g1725895 | 273186.6c | 1830 | 0 | |
| 800 | 493202 | g4057933 | 404509.1 | 2015 | 0 | Human G-protein-coupled receptor GPR-NGA gene, complete cds. |
| 801 | 465848 | g1504234 | 890191.3c | 2187 | 0 | |
| 802 | 653622 | g1826650 | 344071.13 | 1902 | 0 | Human mRNA; cDNA DKFZp434L2015 (from clone DKFZp434L2015); |
| | | | | | | partial cds. |
| 803 | 873690 | g2199651 | 222689.1c | 1655 | 0 | Human pyruvate kinase type L mRNA, complete cds. |
| 804 | 368164 | g1355113 | 125472.24 | 1562 | 1.00E−97 | Human nuclear factor NF45 mRNA, complete cds. |
| 805 | 581834 | g1726099 | | | | |
| 806 | 837565 | g2116248 | | | | |
| 807 | 423772 | g1428470 | 1031520.1 | 1413 | 0 | |
| 808 | 747167 | g1896984 | 221880.2 | 1652 | 8.00E−26 | predicted using Genefinder; cDNA EST yk462d1.5 comes from |
| | | | | | | this gene; cDNA EST yk391f12.5 comes from this gene; |
| | | | | | | cDNA EST CEMSH93R comes from this gene; cDNA EST yk204g10.5 |
| | | | | | | comes from this gene; cDNA EST yk668d11.3 comes from |
| | | | | | | this gene |
| 809 | 571805 | g1660393 | 232313.17 | 1669 | 0 | Human double-stranded RNA-binding nuclear protein |
| | | | | | | NFAR-2 mRNA, partial cds. |
| 810 | 476275 | g1530247 | 351237.1 | 1954 | 0 | |
| 811 | 641051 | g1800965 | 481411.3 | 2133 | 0 | Human cdc25A mRNA, complete cds. |
| 812 | 355445 | g1333443 | | | | |
| 813 | 818653 | g2192789 | 246727.18 | 1785 | 0 | Human HSPC133 mRNA, complete cds. |
| 814 | 933939 | g2306707 | 257576.16 | 1821 | 0 | Human mRNA for novel heterogeneous nuclear RNP protein, |
| | | | | | | L protein. |
| 815 | 671075 | g1865354 | 256011.77 | 1809 | 0 | Human mRNA for protein homologous to elongation factor |
| | | | | | | 1-gamma from A. salina. |
| 816 | 643755 | g1800406 | 244231.11 | 1762 | 0 | Human mRNA for unknown protein of uterine endometrium. |
| 817 | 577312 | g1671585 | 023418.6 | 1381 | 0 | Human putative ATP-dependent mitochondrial RNA helicase |
| | | | | | | (SUV3) mRNA, nuclear gene encoding mitochondrial protein, |
| | | | | | | complete cds. |
| 818 | 439620 | g1464672 | 289947.2 | 1840 | 0 | Human Tax1 binding protein mRNA, partial cds. |
| 819 | 747136 | g1915371 | 367984.1 | 1969 | 7.00E−72 | Human HMG-2 inRN A. |
| 820 | 679015 | g1876763 | | | | |
| 821 | 445310 | g1479957 | 376249.1 | 1974 | 8.00E−31 | egg-specific protein |
| 822 | 385746 | g5600036 | | | | |
| 823 | 699237 | g1876267 | 1169034.2 | 1515 | 0 | Human elongation factor Ts mRNA, nuclear gene encoding |
| | | | | | | mitochondrial protein, complete cds. |
| 824 | 693114 | g1882608 | 197889.2 | 1593 | 3.00E−65 | Human putative outer mitochondrial membrane 34 kDa |
| | | | | | | translocase hTOM34 mRNA, complete cds. |
| 825 | 572614 | g1661715 | 247658.3 | 1787 | 0 | Human aldehyde dehydrogenase E3′ mRNA, partial cds. |
| 826 | 482138 | g1553330 | 1169216.1 | 1516 | 2.00E−31 | hypothetical protein |
| 827 | 317894 | g1287003 | 1143436.1 | 1499 | 0 | Human HSPC160 mRNA, complete cds. |
| 828 | 636513 | g1765705 | 474208.52 | 2092 | 1.00E−92 | Human mRNA for RNA polymerase II hRPB11 subunit. |
| 829 | 620239 | g4289212 | 443251.36 | 2074 | 0 | Human mRNA for ribosomal protein L39, complete cds. |
| 830 | 443413 | g1475020 | | | | |
| 831 | 716630 | g1901484 | 476265.36 | 2117 | 0 | Human p18 protein mRNA, complete cds. |
| 832 | 354910 | g1330620 | 474132.3c | 2090 | 0 | Human Nedd-4-like ubiquitin-protein ligase WWP2 mRNA, |
| | | | | | | complete cds. |
| 833 | 580810 | g1715434 | | | | |
| 834 | 480685 | g1558145 | 1073112.28 | 1459 | 0 | Human thyroid receptor interactor (TRIP1) mRNA, |
| | | | | | | complete cds. |
| 835 | 550910 | g1643472 | 1084760.3 | 1476 | 8.00E−13 | L-asparaginase |
| 836 | 480395 | g4057853 | | | | |
| 837 | 871585 | g2187114 | 1144501.1 | 1505 | 6.00E−29 | Unknown protein |
| 838 | 465403 | g1505274 | 232831.6 | 1674 | 0 | Human SH2-containing protein Nsp2 mRNA, complete cds. |
| 839 | 747059 | g1915247 | 361088.4c | 1962 | 0 | Human 26S proteasome-associated pad1 homolog (POH1) mRNA, |
| | | | | | | complete cds. |
| 840 | 760832 | g2039616 | 253870.6c | 1802 | 0 | Novel Human gene mapping to chomosome 1. |
| 841 | 424329 | g4271845 | 404262.2 | 2012 | 0 | Human mRNA for KIAA0518 protein, partial cds. |
| 842 | 834148 | g2071655 | 815566.6 | 2176 | 0 | Human unknown protein IT12 mRNA, partial cds. |
| 843 | 571406 | g1660535 | 1005811.1 | 1407 | 0 | Human translation initiation factor eIF3 p44 subunit mRNA, |
| | | | | | | complete cds. |
| 844 | 680123 | g1875722 | 109657.9 | 1490 | 0 | Human CCAAT-box-binding factor (CBF) mRNA, complete cds. |
| 845 | 805129 | g2201461 | 337822.11 | 1892 | 0 | Human mRNA for M96A protein. |
| 846 | 576901 | g1681507 | | | | |
| 847 | 336684 | g4258562 | 1185071.1 | 1528 | 0 | Human mRNA for KIAA0595 protein, partial cds. |
| 848 | 577010 | g1681609 | 174363.1 | 1581 | 0 | |
| 849 | 733738 | g1907494 | | | | |
| 850 | 420302 | g1539052 | | | | |
| 851 | 720951 | g1903498 | | | | |
| 852 | 421129 | g4275640 | 252872.1 | 1796 | 7.00E−49 | Rhesus monkey B2 repetitive sequence. |
| 853 | 472332 | g1509475 | 1072451.3 | 1436 | 0 | Human mRNA for GC box bindig protein, complete cds. |
| 854 | 550649 | g1643499 | | | | |
| 855 | 775875 | g1918772 | 814508.1 | 2171 | 0 | |
| 856 | 476610 | g4061243 | | | | |
| 857 | 580935 | g1702122 | 233411.6 | 1686 | 0 | |
| 858 | 571759 | g1660379 | 352406.7 | 1956 | 0 | Human mRNA for osteopontin. |
| 859 | 441394 | g1464991 | 481411.6c | 2134 | 0 | Human cdc25 A mRNA, complete cds. |
| 860 | 763352 | g1910788 | 350412.4 | 1946 | 0 | Human mRNA for villin-like protein, complete cds. |
| 861 | 573743 | g4299578 | 346645.6 | 1918 | 0 | Human mRNA for cytosolic serine hydroxymethyltransferase, |
| | | | | | | clone pUS1206. |
| 862 | 473802 | g1513848 | 1188616.2 | 1541 | 0 | |
| 863 | 535188 | g1629951 | 1170290.3c | 1518 | 0 | Human mRNA for GARS-AIRS-GART. |
| 864 | 388321 | g1379666 | | | | |
| 865 | 316967 | g1286269 | 255816.6 | 1806 | 0 | Human mRNA for muscle specific enolase (MSE) |
| | | | | | | (EC 4.2.1.11). |
| 866 | 314456 | g1285038 | 349989.12 | 1938 | 0.0004 | putative carboxypeptidase |
| 867 | 318263 | g1287290 | | | | |
| 868 | 466254 | g1504699 | 023985.1 | 1382 | 5.00E−62 | Human 26S proteasome ATPase subunit mRNA, complete cds. |
| 869 | 775342 | g2039947 | | | | |
| 870 | 748879 | g1935976 | 480310.59 | 2122 | 0 | Human prefoldin subunit 2 mRNA, complete cds. |
| 871 | 480205 | g4061306 | 350231.5 | 1943 | 0 | Human (clone FBK III 16) protein tyrosine kinase |
| | | | | | | (NET PTK) mRNA, complete cds. |
| 872 | 764803 | g1912793 | 228845.4 | 1658 | 0 | |
| 873 | 481485 | g1553995 | 199098.4 | 1601 | 7.00E−05 | rplQ homologue (identity of 84% to B. sublilis) |
| 874 | 437167 | g4272253 | 086968.3 | 1404 | 0 | Human D13S106 mRNA for a highly charged amino acid |
| | | | | | | sequence. |
| 875 | 698100 | g1863458 | | | | |
| 876 | 618834 | g1748970 | | | | |
| 877 | 574981 | g1684446 | 236100.1 | 1725 | 7.00E−95 | Human CGI-113 protein mRNA, complete cds. |
| 878 | 876655 | g2232384 | 1093970.1 | 1485 | 2.00E−23 | SM-20 |
| 879 | 779876 | g2074938 | 337822.2 | 1893 | 0 | Human PHD finger DNA binding protein isoform 1 (M96) mRNA, |
| | | | | | | alternatively spliced, complete cds. |
| 880 | 437994 | g1463778 | 235247.38 | 1718 | 0 | Human clone H41 unknown mRNA. |
| 881 | 638265 | g1796544 | 333506.1 | 1874 | 0 | Human nuclear FMRP interacting protein 1 (NUFIP1) mRNA, |
| | | | | | | complete cds. |
| 882 | 576572 | g4061800 | 1071477.14 | 1420 | 0 | |
| 883 | 464800 | g1500827 | 804409.14 | 2163 | 0 | Human mRNA for KIAA0281 gene, complete cds. |
| 884 | 803541 | g2201576 | 1075277.1 | 1465 | 2.00E−67 | Human melanoma antigen p15 mRNA, complete cds. |
| 885 | 427295 | g4271974 | 154692.2 | | | |
| 886 | 735617 | g1907914 | 391068.20c | 1981 | 0 | Human mRNA for SAP 130 spliceosomal protein. |
| 887 | 873314 | g2202021 | 1071453.5 | 1418 | 0 | Human pre-mRNA splicing factor (SFRS3) mRNA, complete cds. |
| 888 | 575067 | g4061769 | 400539.18c | 1994 | 0 | Human full length insert cDNA clone ZD65D11. |
| 889 | 437626 | g1447178 | | | | |
| 890 | 353521 | g1325221 | | | | |
| 891 | 642467 | g1776773 | 138528.7 | 1567 | 0 | Human transforming acidic coiled-coil containing |
| | | | | | | protein 3 (TACC3) mRNA, complete cds. |
| 892 | 775135 | g2040007 | 453970.1 | 2080 | 7.00E−48 | p52 pro-apototic protein |
| 893 | 582028 | g1725824 | 1185744.1 | 1531 | 0 | Human SL15 protein mRNA, complete cds. |
| 894 | 523463 | g1619172 | 457998.1 | 2083 | 0 | Human testis-specific chromodomain Y-like protein |
| | | | | | | (CDYL) mRNA, alternatively processed, complete cds. |
| 895 | 459170 | g4057763 | | | | |
| 896 | 367802 | g1355017 | | | | |
| 897 | 337217 | g4258592 | | | | |
| 898 | 748374 | g1915433 | 1011653.1 | 1410 | 0 | Human ribosomal protein L21 mRNA, complete cds. |
| 899 | 737058 | g1910473 | 211575.1c | 1638 | 3.00E−38 | Human mRNA for MTH1b (p22), MTH1c (p21), MTH1d (p18), |
| | | | | | | complete cds. |
| 900 | 576686 | g1682139 | | | | |
| 901 | 874192 | g2200290 | 1072451.3 | 1436 | 0 | Human mRNA for GC box bindig protein, complete cds. |
| 902 | 465052 | g5599406 | 409135.14 | 2029 | 0 | Human mRNA for alkalin phosphatase, complete cds. |
| 903 | 367601 | g1355494 | 202224.1 | 1626 | 0 | Human mRNA for KIAA0156 gene, complete cds. |
| 904 | 464548 | g1497679 | 337870.3c | 1894 | 0 | |
| 905 | 419614 | g1538782 | 480931.2 | 2128 | 7.00E−36 | Human CpG island DNA genomic Mse1 fragment, clone 26g5, |
| | | | | | | forward read cpg26g5.ft1b. |
| 906 | 809055 | g2192192 | | | | |
| 907 | 574939 | g1675885 | 336999.2 | 1890 | 0 | Human zinc finger protein (ZNF143) mRNA, complete eels. |
| 908 | 550845 | g1643555 | 245648.11 | 1775 | 0 | Human lck mRNA for membrane associated protein tyrosine |
| | | | | | | kinase. |
| 909 | 746994 | g1896854 | 361528.3 | 1964 | 2.00E−34 | cerebellar postnatal development protein-1 |
| 910 | 676311 | g1806829 | | | | |
| 911 | 805419 | g2202726 | 475524.1 | 2115 | 0 | Human CLDN6 gene for claudin-6. |
| 912 | 889543 | g2233736 | | | | |
| 913 | 313741 | g1285068 | 202016.6c | 1623 | 0 | Human mRNA; cDNA DKFZp566I0947 (from clone |
| | | | | | | DKEZp566I0947). |
| 914 | 777598 | g1919634 | 332518.2 | 1868 | 0 | Human mRNA for serine/threonine protein kinase SAK. |
| 915 | 573784 | g1662934 | 1074101.1 | 1462 | 9.00E−16 | Human phosphoserine aminotransferase (PSA) mRNA, |
| | | | | | | complete cds. |
| 916 | 721931 | g1904713 | 345268.3 | 1912 | 0 | Human hCMT1c mRNA for mRNA (guanine-7-)methyltransferase, |
| | | | | | | partial cds. |
| 917 | 735607 | g1908557 | 765534.1 | 2158 | 9.00E−31 | hypothetical protein |
| 918 | 532659 | g1575892 | 222798.1 | 1656 | 3.00E−88 | Human HSPC028 mRNA, complete cds. |
| 919 | 761119 | g2040631 | 347294.1 | 1923 | 0 | Human breast cancer susceptibility (BRCA2) mRNA, |
| | | | | | | complete cds. |
| 920 | 653427 | g1864742 | | | | |
| 921 | 354212 | g1328570 | 234908.13 | 1709 | 0 | Human mRNA activated in tumor suppression, clone |
| | | | | | | TSAP13 extended. |
| 922 | 746955 | g1915334 | 1190365.1c | 1559 | 0 | Human CpG island DNA genomic Mse1 fragment, clone 187d3, |
| | | | | | | reverse read cpg187d3.rt1a. |
| 923 | 750782 | g1936808 | 903389.6 | 2211 | 0.001 | contains Pro-rich Px motifs: SPKPP (20X), PEPPA (9X); similar |
| | | | | | | to soybean pro-rich cell wall protein, corresponds to |
| | | | | | | Swiss-Prot Accession Number P13993 |
| 924 | 437517 | g1447055 | 234124.11 | 1700 | 0 | Human protein tyrosine phosphatase (PTPase) mRNA, |
| | | | | | | complete cds. |
| 925 | 425352 | g1435946 | 243474.28 | 1758 | 0 | Human ribosomal protein S4 (RPS4X) isoform mRNA, |
| | | | | | | complete cds. |
| 926 | 427339 | g1446760 | | | | |
| 927 | 480747 | g4273433 | 236654.2 | 1731 | 2.00E−22 | zinc finger protein |
| 928 | 466150 | g1504746 | | | | |
| 929 | 440985 | g1465026 | 228845.9 | 1659 | 0 | Human mRNA for thrombospondin. |
| 930 | 760795 | g2039745 | | | | |
| 931 | 820518 | g2102208 | 346898.5 | 1921 | 0 | Human eukaryotic translation initiation factor (eIF3) mRNA, |
| | | | | | | complete cds. |
| 932 | 775596 | g1918551 | 1189704.1 | 1551 | 0 | |
| 933 | 751500 | g4316979 | 447885.1 | 2078 | 0 | |
| 934 | 439542 | g4315275 | 335511.3 | 1882 | 0.0002 | KIAA0337 |
| 935 | 717469 | g2057407 | | | | |
| 936 | 533117 | g1576271 | 412999.3 | | | |
| 937 | 478374 | g1529455 | 1085430.3 | 1478 | 0 | Human mRNA for KIAA0560 protein, partial cds. |
| 938 | 439902 | g1464733 | 200115.6 | 1610 | 0.0009 | subunit of RNA polymerase II transcription factor TFIID |
| 939 | 874148 | g2199752 | 404705.9c | 2017 | 0 | Human chromosome segregation gene homolog CAS mRNA, |
| | | | | | | complete cds. |
| 940 | 752290 | g1936231 | 233795.1 | 1693 | 6.00E−07 | unknown protein |
| 941 | 443711 | g4056914 | 1144009.2 | 1502 | 2.00E−54 | CGI-89 protein |
| 942 | 574835 | g1684043 | | | | |
| 943 | 408040 | g1538690 | 337894.3 | 1895 | 5.00E−05 | nucleolar phosphoprotein |
| 944 | 476407 | g1529858 | 429681.1 | 2059 | 3.00E−72 | Human protein phosphatase X (PPX) mRNA, complete cds. |
| 945 | 851054 | g2187435 | | | | |
| 946 | 445127 | g1487454 | | | | |
| 947 | 475443 | g4273372 | 067329.4 | 1400 | 0 | |
| 948 | 637237 | g1776625 | | | | |
| 949 | 620195 | g1759202 | 474592.5 | 2102 | 0 | Human leukemia virus receptor 1 (GLVR1) mRNA, complete cds. |
| 950 | 763563 | g1932058 | 1072248.1 | 1431 | 0 | Human histone acetyltransferase (HBO1) mRNA, complete cds. |
| 951 | 572869 | g1662692 | | | | |
| 952 | 640556 | g1800824 | | | | |
| 953 | 427480 | g4271985 | | | | |
| 954 | 831991 | g2157465 | | | | |
| 955 | 418955 | g1542313 | | | | |
| 956 | 419848 | g1538911 | 898228.8 | 2190 | 0 | Human X104 mRNA, complete cds. |
| 957 | 439411 | g1447604 | 236544.2 | 1730 | 0 | |
| 958 | 733810 | g1904886 | 235147.66 | 1711 | 0 | Human mRNA for elongation factor-1-beta. |
| 959 | 385341 | g1385099 | 764831.1 | 2155 | 8.00E−89 | Human full length insert cDNA clone ZD55G12. |
| 960 | 445230 | g4272049 | 378633.7 | 1975 | 0 | Human mRNA for motor protein, complete cds. |
| 961 | 387482 | g1373969 | 236996.6 | 1733 | 0 | Human mRNA for inteferon related IFRD2 (PC4-B) protein |
| | | | | | | (TFRD2 gene), SM15 homologue. |
| 962 | 442585 | g1476397 | 1085501.1 | 1480 | 0 | Human EF-1delta gene encoding Human elongation |
| | | | | | | factor-1-delta. |
| 963 | 438917 | g1463839 | 339157.14 | 1896 | 0 | Human serine/threonine kinase ULK1 (ULK1) mRNA, |
| | | | | | | complete cds. |
| 964 | 406351 | g1540744 | 118726.1 | 1535 | 0 | Human mRNA; cDNA DKFZp434K0410 (from clone DKFZp434K0410); |
| | | | | | | partial cds. |
| 965 | 805842 | g2053335 | | | | |
| 966 | 574832 | g1684038 | 205655.4 | 1634 | 7.00E−09 | predicted using Genefinder; Similarity to Yeast |
| | | | | | | mitochondrial ribosomal protein S5 (SW:RT05_YEAST); |
| | | | | | | cDNA EST EMBL:D65461 comes from this gene; cDNA |
| | | | | | | EST EMBL:D68901 comes from this gene; cDNA |
| | | | | | | EST yk432a4.3 conies from this gene; cDNA |
| | | | | | | EST yk432a4> |
| 967 | 642145 | g1810800 | 1073074.196c | 1453 | 0 | Human mRNA; cDNA DKFZp434P1514 (from clone DKFZp434P1514); |
| | | | | | | partial cds. |
| 968 | 525104 | g4060086 | 274401.5 | 1833 | 0 | Human mRNA for putative lipoic acid synthetase, |
| | | | | | | partial. |
| 969 | 748517 | g1907820 | 331555.1 | 1859 | 0 | Human adenosine kinase mRNA, complete cds. |
| 970 | 409069 | g1541765 | 430224.2 | 2065 | 0 | Human p53 (TP53) gene, complete cds. |
| 971 | 352836 | g4199091 | | | | |
| 972 | 472605 | g1513459 | 237553.1 | 1737 | 1.00E−59 | Human transcription factor ETR101 mRNA, |
| | | | | | | complete cds. |
| 973 | 635025 | g1739192 | | | | |
| 974 | 804482 | g2202569 | | | | |
| 975 | 386101 | g1369148 | 257593.2 | 1823 | 0 | |
| 976 | 474339 | g1514227 | 010794.4 | 1374 | 0 | Human mRNA for TGIF protein. |
| 977 | 598898 | g1746705 | | | | |
| 978 | 478167 | g1529286 | 189289.8 | 1584 | 0 | Human tumorous imaginal discs protein Tid56 homolog |
| | | | | | | (TID1) mRNA, complete cds. |
| 979 | 733726 | g1905098 | 349663.16 | 1934 | 0 | Human clone IMAGE Consortium 302831 latent transforming |
| | | | | | | growth factor-beta binding protein 4 mRNA, partial cds. |
| 980 | 407997 | g1538669 | 1144078.2 | 1503 | 5.00E−08 | proline-rich mucin homolog |
| 981 | 475670 | g4061215 | | | | |
| 982 | 467501 | g4057800 | | | | |
| 983 | 643463 | g1772281 | | | | |
| 984 | 574251 | g4444704 | 228150.48 | 1657 | 0 | Human RPL13A, U32, U33, U34, U35, RPS11, U35 genes for |
| | | | | | | ribosomal protein L13a and S11, U32, U33, U34, U35, and |
| | | | | | | U35 snoRNA, complete cds and sequence. |
| 985 | 476205 | g1530366 | | | | |
| 986 | 457150 | g4057750 | | | | |
| 987 | 465903 | g1504656 | 247977.4c | 1788 | 9.00E−53 | Human CI-B14.5a homolog mRNA, complete cds. |
| 988 | 659928 | g1840271 | 040038.51 | 1394 | 0 | Human mRNA for ribosomal protein L35a. |
| 989 | 573609 | g1676392 | 1072876.2 | 1441 | 0 | Human mRNA for KIAA0253 gene, partial cds. |
| 990 | 736069 | g1908611 | 202489.22 | 1628 | 0 | Human zinc finger/leucine zipper protein (AF10) mRNA, |
| | | | | | | complete cds. |
| 991 | 720870 | g1903917 | 899209.8c | 2196 | 0 | Human mRNA for erythrocyte cytosolic protein of 51 kDa. |
| 992 | 468633 | g1475438 | 252872.2 | 1797 | 9.00E−23 | Rhesus monkey B2 repetitive sequence. |
| 993 | 575554 | g4299879 | 399465.4 | 1988 | 0 | Human mRNA for KIAA0136 gene, partial cds. |
| 994 | 640177 | g1793165 | | | | |
| 995 | 834934 | g2193407 | | | | |
| 996 | 400741 | g4256936 | 1171111.5 | 1519 | 0 | Human colin carcinoma laminin-binding protein mRNA, |
| | | | | | | complete cds. |
| 997 | 475814 | g4061223 | 444662.7 | 2075 | 2.00E−39 | similar GAR1 related proteins (small nucleolar protein |
| | | | | | | required for pre-rRNA splicing) |
| 998 | 808377 | g2192845 | | | | |
| 999 | 583869 | g1715201 | 232279.4 | 1668 | 0 | Human mRNA for KIAA1177 protein, partial cds. |
| 1000 | 478383 | g4061346 | 1079863.1 | 1472 | 2.00E−65 | Human methylenetetrahydrofolate dehydrogenase- |
| | | | | | | methenyltetrahydrofolate cyclohydrolase-formyltetrahydrofolate |
| | | | | | | synthetase mRNA, complete cds. |
| 1001 | 763597 | g1932074 | 200727.3c | 1612 | 1.00E−08 | contains similarity to several apoptosis or programmed |
| | | | | | | cell death proteins such as rat apoptosis protein |
| | | | | | | RP-8 (GB:M80601) |
| 1002 | 659193 | g1827385 | 060445.3 | | | |
| 1003 | 571278 | g1660261 | 256051.39 | 1812 | 0 | Human RNA-bindiag protein regulatory subunit mRNA, |
| | | | | | | complete cds. |
| 1004 | 535199 | g1630005 | 407870.4 | 2026 | 0 | Human coiled-coil related protein DEEPEST (DEEPEST) |
| | | | | | | mRNA, complete cds. |
| 1005 | 696858 | g1838388 | 902879.1 | 2206 | 0 | Human mRNA for HL23 ribosomal protein homologue. |
| 1006 | 850354 | g2164878 | 347805.31 | 1925 | 0 | Human mRNA for RPD3 protein, complete cds. |
| 1007 | 466295 | g1504818 | 241630.21 | 1752 | 0 | Human mRNA for ribosomal protein (homologuous to |
| | | | | | | Yeast S24). |
| 1008 | 619385 | g1751569 | | | | |
| 1009 | 425049 | g1435873 | | | | |
| 1010 | 579349 | g4058103 | | | | |
| 1011 | 762516 | g1909864 | 368651.1 | 1970 | 0 | Human gene for PP15 (placental protein 15). |
| 1012 | 676748 | g1811238 | 480496.13 | 2125 | 0 | |
| 1013 | 483333 | g1555497 | 236142.6 | 1726 | 0 | Human medium-chain acyl-CoA dehydrogenase (ACADM) mRNA, |
| | | | | | | complete cds. |
| 1014 | 850426 | g2164943 | 903951.15 | 2217 | 0 | ArgRS = arginyl-tRNA synthetase [Human, |
| | | | | | | ataxia-telangiectasia patients, EBV-lymphoblastoid cells, |
| | | | | | | mRNA, 2120 nt]. |
| 1015 | 405779 | g1540353 | | | | |
| 1016 | 651274 | g1814515 | 902159.16 | 2201 | 0 | Human proteasome subunit XAPC7 mRNA, complete cds. |
| 1017 | 481576 | g4604547 | 344598.3 | 1907 | 0 | Human cytochrome oxidase assembly factor (PET112) mRNA, |
| | | | | | | complete cds. |
| 1018 | 735773 | g4317871 | 175394.1 | 1583 | 0 | |
| 1019 | 722623 | g1897138 | 010794.1 | 1373 | 0 | Human mRNA for TGIF protein. |
| 1020 | 318642 | g1285873 | 893157.1 | 2188 | 0 | Human L23 mRNA for putative ribosomal protein. |
| 1021 | 595972 | g1699453 | 359995.18 | 1959 | 0 | Human mRNA; cDNA DKFZp434L1613 (from clone DKFZp434L1613); |
| | | | | | | partial cds. |
| 1022 | 761158 | g2040800 | 398965.2 | 1986 | 0 | Human mRNA encoding RAMP3. |
| 1023 | 949722 | g2283423 | 349293.3 | 1930 | 0 | Human mRNA for KIAA1261 protein, partial cds. |
| 1024 | 766582 | g1915467 | 903243.2 | 2210 | 0 | Human glutathione transferase (GSTA3) mRNA, partial cds. |
| 1025 | 480329 | g1550921 | 902858.2 | | | |
| 1026 | 465744 | g1504292 | 331520.8 | 1858 | 0 | Human mRNA; cDNA DKFZp566D143 (from clone DKFZp556D143); |
| | | | | | | partial cds. |
| 1027 | 481242 | g1554197 | 237495.4 | 1736 | 0 | Human Fas associated factor 1 mRNA, complete cds. |
| 1028 | 577501 | g4061820 | 1190255.1c | 1558 | 9.00E−56 | Human HepG2 3′ region cDNA, clone hmd2g04. |
| 1029 | 763424 | g1910590 | 1189812.1 | 1554 | 0 | Human cysteine and glycine-rich protein 2 (CSRP2) mRNA, |
| | | | | | | complete cds. |
| 1030 | 408693 | g1541609 | 238614.9 | 1744 | 0 | Human small nuclear RNA protein E mRNA, complete cds. |
| 1031 | 480196 | g1550881 | 1186614.1c | 1534 | 0 | Human CC chemokine gene cluster, complete sequence. |
| 1032 | 818256 | g2140196 | 814647.3 | 2174 | 0 | Human HALPHA44 gene for alpha-tubulin, exons 1-3. |
| 1033 | 445439 | g1481110 | | | | |
| 1034 | 493683 | g1630415 | 1071820.3 | 1428 | 0 | Human mRNA; cDNA DKFZp434P1215 (from clone DKFZp434P1215); |
| | | | | | | partial cds. |
| 1035 | 642165 | g1810961 | 233723.16 | 1690 | 0 | |
| 1036 | 641368 | g1796686 | | | | |
| 1037 | 425857 | g1436705 | 447819.1 | 2077 | 2.00E−59 | alanine aminotransferase |
| 1038 | 809206 | g2139623 | 230408.9 | 1663 | 0 | Human HSPC179 mRNA, complete cds. |
| 1039 | 933183 | g2284211 | 232279.11 | 1667 | 0 | Human mRNA for KIAA1177 protein, partial cds. |
| 1040 | 748639 | g1908078 | | | | |
| 1041 | 747180 | g1896883 | | | | |
| 1042 | 385358 | g1380989 | 480452.6c | 2123 | 0 | Human mRNA for protein D123, complete cds. |
| 1043 | 872986 | g2192375 | | | | |
| 1044 | 329816 | g1288172 | 246387.2 | 1783 | 0 | Human NTF2-related export protein NXT1 (NXT1) mRNA, |
| | | | | | | complete cds. |
| 1045 | 672656 | g1853779 | | | | |
| 1046 | 617288 | g4298653 | | | | |
| 1047 | 423327 | g4271809 | 332777.8 | 1873 | 0 | Human mRNA for KIAA1259 protein, partial cds. |
| 1048 | 459094 | g4272149 | | | | |
| 1049 | 834917 | g2193409 | | | | |
| 1050 | 735089 | g1896247 | | | | |
| 1051 | 455483 | g1484291 | 246336.9 | 1782 | 0 | Human geranylgeranyl trartsferase type II beta-subunit |
| | | | | | | mRNA, complete cds. |
| 1052 | 479759 | g4061296 | 460283.5c | 2085 | 0 | Human mRNA for KIAA0274 gene, complete cds. |
| 1053 | 778381 | g2073712 | 385182.12 | 1979 | 0 | Human barrier-to-autointegration factor mRNA, |
| | | | | | | complete cds. |
| 1054 | 473404 | g1513959 | 350905.4 | 1951 | 0 | Human Treacher Collins syndrome (TCOF1) mRNA, |
| | | | | | | complete cds. |
| 1055 | 406889 | g1541054 | 760699.15 | 2148 | 0 | Human RSU-1/RSP-1 mRNA, complete cds. |
| 1056 | 351154 | g1325565 | 197067.3 | 1589 | 0 | Human SnRNP core protein Sm D2 mRNA, complete cds. |
| 1057 | 660756 | g1827284 | 1085450.1 | 1479 | 0 | neurofilament-66 [Human, fetal brain, mRNA, |
| | | | | | | 3197 nt]. |
| 1058 | 678992 | g1875229 | 813771.1 | 2169 | 0 | Human (clone p5-23-3) mRNA. |
| 1059 | 441983 | g1476453 | 021846.3 | 1380 | 0 | Human HSPC015 mRNA, complete cds. |
| 1060 | 483476 | g4273580 | | | | |
| 1061 | 699466 | g1876365 | 1189599.1 | 1550 | 1.00E−70 | Human mRNA for Lsm5 protein. |
| 1062 | 464381 | g1497540 | 344603.4 | 1908 | 0 | Human mRNA; cDNA DKFZp586K0919 (from clone DKFZp586K0919); |
| | | | | | | complete cds. |
| 1063 | 736584 | g1908983 | | | | |
| 1064 | 903419 | g2259182 | 255824.44 | 1807 | 0 | Human aldolase A mRNA, complete cds. |
| 1065 | 607469 | g1751918 | 237409.8 | 1734 | 0 | Human purine nucleoside phosphorylase gene, exons 3, 4 and 5. |
| 1066 | 775067 | g2040315 | 903115.9 | 2209 | 0 | Human phosphomannomutase mRNA, complete cds. |
| 1067 | 803817 | g2101798 | 1072439.1 | 1435 | 0 | Human HSPC071 mRNA, complete cds. |
| 1068 | 438774 | g1530821 | 335173.1 | 1881 | 0 | Human carbon catabolite repression 4 protein homolog mRNA, |
| | | | | | | complete cds. |
| 1069 | 317976 | g1285920 | 145503.16 | 1569 | 0 | Human sorting nexin 5 (SNX5) mRNA, complete cds. |
| 1070 | 903284 | g2259542 | 1169639.1 | 1517 | 2.00E−17 | Human CpG island DNA genomic Mse1 fragment, clone 134c10, |
| | | | | | | forward read cpg134c10.ft1a. |
| 1071 | 791433 | g2075599 | | | | |
| 1072 | 476999 | g1527784 | 244097.3 | | | |
| 1073 | 477900 | g1528332 | 898830.7 | 2193 | 0 | Human pescadillo mRNA, complete cds. |
| 1074 | 749569 | g2041641 | 351159.3 | 1953 | 0 | Human DEAD box RNA helicase-like protein mRNA, |
| | | | | | | complete cds. |
| 1075 | 481469 | g4272199 | | | | |
| 1076 | 480026 | g1531178 | 274401.21c | 1832 | 0 | Human mRNA for KIAA1089 protein, partial cds. |
| 1077 | 386766 | g1372077 | 247977.4c | 1788 | 9.00E−53 | Human CI-B14.5a homolog mRNA, complete cds. |
| 1078 | 693287 | g2050114 | 221819.3 | 1650 | 0 | Human von Hippel-Lindau binding protein (VBP-1) mRNA, |
| | | | | | | complete cds. |
| 1079 | 404268 | g5599332 | | | | |
| 1080 | 386122 | g1369154 | | | | |
| 1081 | 478301 | g1529366 | 348196.18 | 1929 | 0 | Human 4F2 glycosylated heavy chain (4F2HC) antigen gene, |
| | | | | | | exon 1 and 2. |
| 1082 | 576570 | g1677315 | 124921.6 | 1561 | 0 | Human IEF SSP 9502 mRNA, complete cds. |
| 1083 | 466413 | g1506394 | | | | |
| 1084 | 418473 | g1541855 | 1143289.1 | 1498 | 0 | Human HSPC260 mRNA, partial cds. |
| 1085 | 636207 | g1766849 | 462958.8 | 2086 | 0 | Human class III alcohol dehydrogenase (ADH5) chi |
| | | | | | | subunit mRNA, complete cds. |
| 1086 | 577180 | g1682379 | 330919.4 | 1852 | 1.00E−11 | cytoplasmic linker protein CLIP-170 |
| 1087 | 459668 | g1493594 | 331018.14 | 1854 | 0 | Human chaperonin containing t-complex polypeptide 1, |
| | | | | | | delta subunit (Cctd) mRNA, complete cds. |
| 1088 | 746463 | g1896502 | 253235.1 | 1798 | 6.00E−74 | Human mRNA for DNA primase (subunit p48). |
| 1089 | 820355 | g2102111 | 1143532.1 | 1500 | 1.00E−18 | Human translation initiation factor eIF3 p66 subunit mRNA, |
| | | | | | | complete cds. |
| 1090 | 761127 | g2040635 | | | | |
| 1091 | 467873 | g1509701 | | | | |
| 1092 | 777779 | g1932483 | 221834.6 | 1651 | 0 | BcDNA.LD18761 |
| 1093 | 372421 | g1357464 | 235314.2 | 1719 | 0 | Human basic transcription factor 2 p44 (btf2p44) gene, |
| | | | | | | partial cds, neuronal apoptosis inhibitory protein (naip) |
| | | | | | | and survival motor neuron protein (smn) genes, |
| | | | | | | complete cds. |
| 1094 | 572285 | g1662010 | 437025.16 | 2070 | 0 | Human mRNA for macropain subunit zeta. |
| 1095 | 571569 | g4061378 | 401484.3 | 2000 | 0 | Human mRNA for proteasome subunit HsC10-II, complete cds. |
| 1096 | 388936 | g1375971 | 391068.20c | 1981 | 0 | Human mRNA for SAP 130 spliceosomal protein. |
| 1097 | 633261 | g1759726 | 404508.1 | | | |
| 1098 | 793038 | g2156889 | 060356.7 | 1398 | 0 | Human mRNA for herpesvirus associated ubiquitin-specific |
| | | | | | | protease (HAUSP). |
| 1099 | 736328 | g1908947 | 1073050.1 | 1452 | 0 | Human deoxyhypusine synthase 2 mRNA, complete cds. |
| 1100 | 348123 | g4199071 | 407870.9c | 2027 | 0 | Human mRNA; cDNA DKFZp434H1210 (from clone DKFZp434H1210); |
| | | | | | | partial cds. |
| 1101 | 482885 | g4273555 | | | | |
| 1102 | 401741 | g1388813 | 889830.1 | 2186 | 0 | |
| 1103 | 679007 | g1875341 | 332516.9c | 1867 | 0 | Human squamous cell carcinama of esophagus mRNA for |
| | | | | | | GRB-7 SH2 domain protein, complete cds. |
| 1104 | 717387 | g2057367 | 474316.9 | 2095 | 0 | Human clone 23783 mRNA sequence. |
| 1105 | 721532 | g1904392 | 474711.5 | 2105 | 0.001 | RPGR protein |
| 1106 | 439254 | g1447384 | | | | |
| 1107 | 808083 | g2157030 | 344071.13 | 1902 | 0 | Human mRNA; cDNA DKFZp434L2015 (from clone DKFZp434L2015); |
| | | | | | | partial cds. |
| 1108 | 476163 | g1530276 | 1189576.1 | 1549 | 0 | Human branched chain alpha-keto acid dehydrogenase (BCKDHB) |
| | | | | | | E1-beta subunit mRNA, complete cds. |
| 1109 | 440715 | g1465189 | | | | |
| 1110 | 636888 | g1766814 | 400607.2c | 1998 | 2.00E−77 | Human FK506-binding protein 25 (FKBP25) mRNA, |
| | | | | | | complete cds. |
| 1111 | 746853 | g1915241 | | | | |
| 1112 | 418606 | g1429121 | | | | |
| 1113 | 336021 | g1318304 | 378633.7 | 1975 | 0 | Human mRNA for motor protein, complete cds. |
| 1114 | 403220 | g1539385 | | | | |
| 1115 | 524367 | g1563312 | | | | |
| 1116 | 351288 | g1324854 | 1074529.1 | 1464 | 6.00E−29 | Human mRNA for KIAA0778 protein, partial cds. |
| 1117 | 401221 | g1428051 | | | | |
| 1118 | 748061 | g1915292 | 475248.5 | 2112 | 7.00E−88 | similar to leucyl-tRNA synthetase; cDNA EST EMBL:D64208 |
| | | | | | | comes from this gene; cDNA EST EMBL:D65056 comes from |
| | | | | | | this gene; cDNA EST MBL:D68348 comes from this gene; |
| | | | | | | cDNA EST yk377h12.3 comes from this gene; cDNA EST |
| | | | | | | yk377h12.5 comes from this g> |
| 1119 | 831799 | g2157099 | 407574.5 | 2024 | 0 | Human ribonuclease P protein subunit p40 (RPP40) gene, |
| | | | | | | complete cds. |
| 1120 | 374877 | g1369506 | 347805.36 | 1926 | 0 | Human histone deacetylase HD1 mRNA, complete cds. |
| 1121 | 534132 | g1594370 | 237495.3 | 1735 | 0 | Human CGI-03 protein mRNA, complete cds. |
| 1122 | 574459 | g4284012 | 238296.6 | 1742 | 0 | Human mRNA for HMGBCG protein. |
| 1123 | 444027 | g1475778 | 244633.5 | 1768 | 2.00E−45 | Human mitochondrial HSP75 mRNA, complete cds. |
| 1124 | 791176 | g2075474 | 903920.6 | 2216 | 0 | Human origin recognition complex subunit 6 (ORC6) mRNA, |
| | | | | | | complete cds. |
| 1125 | 421766 | g4275680 | | | | |
| 1126 | 423713 | g4271832 | 760699.13 | 2147 | 0 | Human RSU-1/RSP-1 mRNA, complete cds. |
| 1127 | 778447 | g2073832 | 342283.7 | 1898 | 0 | Human NY-REN-45 antigen mRNA, complete cds. |
| 1128 | 467830 | g1509690 | | | | |
| 1129 | 617197 | g1755906 | 1004597.1 | 1406 | 0 | |
| 1130 | 793127 | g2142796 | 244382.2 | 1764 | 0 | Human ribosomal protein L9 mRNA, complete cds. |
| 1131 | 779315 | g2074235 | 793531.1 | 2160 | 0 | Human mRNA for KIAA0111 gene, complete cds. |
| 1132 | 427222 | g1446540 | 898830.2c | 2192 | 0 | Human CST gene for cerebroside sulfotransferase, exon |
| | | | | | | 1, 2, 3, 4, 5. |
| 1133 | 807957 | g2116355 | 732479.1 | 2140 | 2.00E−57 | /prediction = (method: |
| 1134 | 820560 | g2102295 | | | | |
| 1135 | 571841 | g4274762 | | | | |
| 1136 | 597896 | g4060437 | 244612.2 | 1767 | 2.00E−95 | Human A1S9 mRNA for A1S9 protein of unknown function. |
| 1137 | 388245 | g1375543 | 816212.1 | 2180 | 0 | Human DNA ligase I mRNA, complete cds. |
| 1138 | 679235 | g1876797 | 174032.1 | 1577 | 0 | |
| 1139 | 874541 | g4614274 | 331937.4 | 1864 | 3.00E−12 | NTR |
| 1140 | 762237 | g1917885 | | | | |
| 1141 | 401167 | g1427988 | 400607.14 | 1996 | 0 | Human mRNA for KIAA0589 protein, partial cds. |
| 1142 | 335017 | g1537473 | 331114.4 | 1856 | 0 | Human mRNA for KIAA0314 gene, partial cds. |
| 1143 | 401821 | g1388915 | 401887.47 | 2009 | 0 | Human mRNA for KIAA1273 protein, partial cds. |
| 1144 | 425452 | g4061573 | 1189743.1 | 1553 | 0 | |
| 1145 | 676921 | g1811610 | 238242.21c | 1741 | 0 | Human hASNA-I mRNA, complete cds. |
| 1146 | 735374 | g4765142 | | | | |
| 1147 | 480488 | g1558048 | 350181.7 | 1942 | 5.00E−69 | Human threonyl-tRNA synthetase mRNA, complete cds. |
| 1148 | 442843 | g1476028 | 350106.12 | 1941 | 0 | Human glutamate dehydrogenase gene, complete cds. |
| 1149 | 406282 | g1540681 | 400837.9 | 1999 | 0 | Human splicing factor, atginine/serine-rich 7 (SERS7) |
| | | | | | | gene, complete cds. |
| 1150 | 619836 | g1759124 | | | | |
| 1151 | 440053 | g4058501 | 1178685.1 | 1525 | 0 | Human partial mRNA for putative eIF2 alpha kinase |
| | | | | | | (GCN2 gene). |
| 1152 | 426529 | g1445840 | 220551.1 | 1644 | 0 | Human origin recognition complex subunit 6 (ORC6) mRNA, |
| | | | | | | complete cds. |
| 1153 | 406088 | g1540555 | 198907.11 | 1600 | 0 | Human mRNA for putative ABC transporter, partial. |
| 1154 | 440194 | g1478815 | 337870.3c | 1894 | 0 | |
| 1155 | 467669 | g4060664 | 480462.1 | 2124 | 0 | Human C-terminal binding protein 2 mRNA, complete cds. |
| 1156 | 406154 | g1540601 | 1041769.1 | 1415 | 7.00E−13 | putative amidohydrolase |
| 1157 | 465520 | g1504514 | 373719.5 | 1973 | 0 | Human ADP/ATP carrier protein mRNA, complete cds. |
| 1158 | 439033 | g1464229 | 1173266.1 | 1523 | 0 | Human bisphosphate 3′-nucleotidase mRNA, complete cds. |
| 1159 | 639451 | g1795853 | 221390.8 | 1647 | 8.00E−47 | Human CpG island DNA genomic Mse1 fragment, done 91b2, |
| | | | | | | forward read cpg91b2.ft1a. |
| 1160 | 355454 | g1333441 | | | | |
| 1161 | 354155 | g1328412 | | | | |
| 1162 | 619401 | g1756740 | 246762.1 | 1786 | 0 | |
| 1163 | 480204 | g1550998 | 272721.32 | 1829 | 5.00E−06 | predicted using Genefinder; cDNA EST yk551g5.3 comes |
| | | | | | | from this gene |
| 1164 | 468792 | g1509016 | 1182186.1 | 1526 | 0 | Human CDC2 gene involved in cell cycle control. |
| 1165 | 573582 | g1676863 | 407488.2 | 2023 | 6.00E−71 | Human mRNA for KIAA0956 protein, partial cds. |
| 1166 | 464447 | g1497572 | 336738.1 | 1885 | 0 | Human Bloom's syndrome protein (BLM) mRNA, complete cds. |
| 1167 | 388091 | g1380598 | 263336.9c | 1827 | 0 | Human mRNA for alanyl-tRNA synthetase, complete cds. |
| 1168 | 576353 | g1677104 | 1133155.1 | 1495 | 0 | Human NNX3 (C19orf2) mRNA, complete cds. |
| 1169 | 475796 | g1529806 | 197563.7 | 1591 | 0 | Human mRNA for KIAA0631 protein, partial cds. |
| 1170 | 834541 | g2192898 | 209635.5 | 1637 | 0 | Human CAD mRNA for multifunctional protein CAD, |
| | | | | | | complete cds. |
| 1171 | 573301 | g1676207 | | | | |
| 1172 | 524361 | g4058216 | 314873.5 | 1846 | 0 | Human DNA recombination and repair protein (MRE11B) mRNA, |
| | | | | | | complete cds. |
| 1173 | 848702 | g2158118 | 765792.1 | 2159 | 2.00E−51 | Human PTD017 mRNA, complete cds. |
| 1174 | 422188 | g1427355 | 249443.1 | 1791 | 9.00E−93 | Human mRNA for E2F-4 protein. |
| 1175 | 422167 | g1427225 | 291494.1 | 1841 | 0 | Human bright and dead ringer gene product homologous protein |
| | | | | | | Bdp mRNA, complete cds. |
| 1176 | 596719 | g1715941 | 1188194.6 | 1536 | 0 | Human mRNA; cDNA DKFZp564G092 (from clone DKFZp564G092); |
| | | | | | | partial cds. |
| 1177 | 427594 | g1445982 | 235749.9 | 1721 | 0 | |
| 1178 | 933852 | g2305399 | 234088.1 | 1699 | 1.00E−74 | /prediction = (method: |
| 1179 | 407354 | g1542220 | 201424.1 | 1619 | 0 | Human HSPC037 protein mRNA, complete cds. |
| 1180 | 424021 | g1542780 | | | | |
| 1181 | 332396 | g1282795 | 1073934.1 | 1461 | 0 | Human mRNA for Pirin, isolate 17. |
| 1182 | 422799 | g1427033 | 1071969.5 | 1430 | 0 | Human mRNA for cell cycle gene RCC1. |
| 1183 | 440795 | g1465253 | 1171439.1 | 1520 | 0 | Human 60S acidic ribosomal protein PO mRNA, complete cds. |
| 1184 | 535409 | g4308335 | | | | |
| 1185 | 441235 | g1465225 | 1189175.1 | 1546 | 0 | Human protein arginine N-methyltransferase 3 (PRMT3) mRNA, |
| | | | | | | partial cds. |
| 1186 | 437602 | g1447172 | 202075.4 | 1624 | 0 | Human phosphatidylinositol(3,4,5)trisphosphate binding |
| | | | | | | protein p42IP4 mRNA, complete cds. |
| 1187 | 874591 | g2200428 | 474418.2 | 2098 | 0 | Human HS1 binding protein HAX-1 mRNA, nuclear gene |
| | | | | | | encoding mitochondrial protein, complete cds. |
| 1188 | 583932 | g1725561 | | | | |
| 1189 | 640682 | g1800372 | 350016.4 | 1939 | 3.00E−48 | Human clone 23585 mRNA sequence. |
| 1190 | 580623 | g1701915 | 236260.1 | 1727 | 0 | |
| 1191 | 67901 1 | g1876761 | 234639.5 | 1707 | 0 | Human putative nucleotide binding protein mRNA, |
| | | | | | | complete cds. |
| 1192 | 482910 | g4061421 | 215053.3 | 1642 | 2.00E−12 | Human CpG island DNA genomic Mse1 fragment, clone 151f3, |
| | | | | | | reverse read cpg151f3.rt1a. |
| 1193 | 373854 | g4257160 | | | | |
| 1194 | 335746 | g1297406 | 199944.6 | 1605 | 0 | Human RNA polymerase I subunit hRPA39 mRNA, complete cds. |
| 1195 | 579715 | g1680952 | 1073079.41 | 1455 | 0 | Human liver mRNA for glyceraldehyde-3-phosphate |
| | | | | | | dehydrogenase (G3PD, EC 1.2.1.12). |
| 1196 | 439548 | g1447669 | 1145579.1 | 1506 | 2.00E−48 | Human mRNA for Lysyl tRNA Synthetase, complete cds. |
| 1197 | 476123 | g1530066 | 346653.9 | 1919 | 0 | Human DNA polymerase alpha mRNA, complete cds. |
| 1198 | 524442 | g4060069 | 1188285.1 | 1538 | 0 | Human mRNA for ribosomal protein L7. |
| 1199 | 533415 | g4060866 | 211628.2 | 1639 | 0 | Human DEAD box RNA helicase Gemin3 mRNA, complete cds. |
| 1200 | 894135 | g2256434 | 1189159.1 | 1545 | 0 | Human CGI-07 protein mRNA, complete cds. |
| 1201 | 422667 | g1427746 | 1072334.1 | 1432 | 0 | Human PTD007 mRNA, complete cds. |
| 1202 | 633209 | g1759709 | 1185605.1c | 1530 | 0 | Human rabkinesin6 mRNA, complete cds. |
| 1203 | 961094 | g4401581 | 815596.1 | | | |
| 1204 | 833661 | g2192978 | 332056.2 | 1865 | 0 | Human KIAA0432 mRNA, complete cds. |
| 1205 | 385901 | g1369058 | 469699.3 | 2087 | 0 | Human sirtuin type 1 (SIRT1) mRNA, complete cds. |
| 1206 | 760921 | g4316563 | | | | |
| 1207 | 775645 | g1918571 | 1086066.1 | 1482 | 0 | Human ribosomal protein S5 mRNA, complete cds. |
| 1208 | 388212 | g1375493 | 234313.1 | 1703 | 1.00E−96 | Human clone 24804 mRNA sequence. |
| 1209 | 441968 | g1475584 | 237553.1 | 1737 | 1.00E−59 | Human transcription factor ETR101 mRNA, complete cds. |
| 1210 | 576156 | g1677291 | 346707.6 | 1920 | 0 | Human mRNA for KIAA0007 gene, partial cds. |
| 1211 | 790122 | g2041159 | 1094883.1 | 1489 | 0 | |
| 1212 | 920055 | g2234893 | 1166415.1 | 1513 | 0 | Human cyclophilin-related processed pseudogene. |
| 1213 | 775189 | g2040164 | 451018.1 | 2079 | 3.00E−11 | Human mRNA for KIAA0631 protein, partial cds. |
| 1214 | 570533 | g1659855 | 1073107.67 | 1458 | 0 | Human mRNA for ribosomal protein L3. |
| 1215 | 716896 | g1902413 | 415052.2 | 2044 | 0 | Human mRNA for ribosomal protein L8. |
| 1216 | 550869 | g1643563 | 360645.13 | 1961 | 0.0006 | cDNA EST yk255b9.3 comes from this gene; cDNA EST yk255b9.5 |
| | | | | | | comes from this gene; cDNA EST EMBL:M75923 comes from this gene |
| 1217 | 792736 | g4320734 | | | | |
| 1218 | 948894 | g2306022 | 234618.5 | 1705 | 0 | Human p85Mcm mRNA. |
| 1219 | 779257 | g2158458 | 261069.1 | 1826 | 3.00E−97 | Human nucleolar protein p40 mRNA, complete cds. |
| 1220 | 765841 | g1915853 | 479908.35 | 2121 | 0 | |
| 1221 | 355808 | g1332851 | | | | |
| 1222 | 437761 | g1463860 | | | | |
| 1223 | 680147 | g1861916 | 400428.2 | 1993 | 0 | Human mRNA for DNA primase (subunit p58). |
| 1224 | 352409 | g1315739 | 201145.5 | 1616 | 0 | Human DEAD-box protein abstrakt (ABS) mRNA, complete cds. |
| 1225 | 388296 | g4613966 | | | | |
| 1226 | 387227 | g1374141 | 408351.1 | 2028 | 5.00E−57 | Human mRNA for LAK-4p, complete cds. |
| 1227 | 329726 | g1286049 | 1078216.1 | 1469 | 1.00E−97 | Macaque mRNA for alpha-tubulin. |
| 1228 | 779598 | g2073952 | 060356.7 | 1398 | 0 | Human mRNA for herpesvirus associated ubiquitin-specific |
| | | | | | | protease (HAUSP). |
| 1229 | 457567 | g4276631 | 1184163.1c | 1527 | 0 | Human mRNA for KIAA0888 protein, partial cds. |
| 1230 | 407274 | g1541250 | | | | |
| 1231 | 314236 | g1284324 | 1165545.1 | 1511 | 2.00E−35 | similar to alpha/beta hydrolase fold; cDNA EST EMBL:T02320 |
| | | | | | | comes from this gene |
| 1232 | 834712 | g2193193 | 205348.2 | 1632 | 4.00E−09 | sconB |
| 1233 | 643854 | g1800840 | 255261.31 | 1804 | 0 | Human alpha enolase mRNA, complete cds. |
| 1234 | 720554 | g1895480 | | | | |
| 1235 | 387946 | g1375726 | 256026.1 | 1810 | 0 | Human karyopherin beta 3 mRNA, complete cds. |
| 1236 | 478417 | g1529491 | 343938.1 | 1901 | 3.00E−23 | Human CpG island DNA genomic Mse1 fragment, clone 68g2, |
| | | | | | | forward read cpg68g2.ft1a. |
| 1237 | 532770 | g1576204 | 1073101.5 | 1457 | 0 | Human ribosomal protein L10 mRNA, complete cds. |
| 1238 | 573725 | g1676454 | 242626.4 | 1755 | 0 | Human mRNA for nuclear factor IV. |
| 1239 | 445567 | g1481167 | 331114.4 | 1856 | 0 | Human mRNA for KIAA0314 gene, partial cds. |
| 1240 | 804963 | g2050203 | | | | |
| 1241 | 574381 | g1677144 | 1164111.1 | 1509 | 0 | Human heat shock protein hsp70-related protein mRNA, |
| | | | | | | complete cds. |
| 1242 | 438125 | g1464002 | 245648.12 | 1776 | 0 | Human T-cell-specific mRNA (YT16) for PTK homologous |
| | | | | | | protein PTK = protein-tyrosine kinase. |
| 1243 | 372368 | g1357434 | | | | |
| 1244 | 535036 | g1595304 | 457674.1 | 2082 | 0 | Human IGF-II mRNA-binding protein 1 (IMP-1) mRNA, |
| | | | | | | complete cds. |
| 1245 | 699417 | g1876237 | 761531.1 | 2150 | 0 | Human mRNA for ribosomal protein, complete cds. |
| 1246 | 444918 | g1478999 | 330801.4 | 1850 | 0 | Human mRNA for HsMcm6, complete cds. |
| 1247 | 775603 | g1918411 | 248870.1 | 1790 | 0 | Human mRNA for leucine rich protein. |
| 1248 | 831643 | g2192227 | 253358.2 | 1799 | 2.00E−77 | Human mRNA for histone H2A.Z. |
| 1249 | 403681 | g1539725 | | | | |
| 1250 | 331974 | g1282346 | 239973.5 | 1749 | 0 | Human mRNA for KIAA0020 gene, complete cds. |
| 1251 | 331901 | g1282303 | | | | |
| 1252 | 642977 | g1768522 | 201253.1 | 1617 | 0 | Human putative ATP/GTP-binding protein (HEAB) mRNA, |
| | | | | | | complete cds. |
| 1253 | 836311 | g4614140 | | | | |
| 1254 | 932884 | g2288304 | 1071666.2 | 1425 | 0 | Human mRNA; cDNA DKFZp564H2023 (from clone DKFZp564H2023). |
| 1255 | 439731 | g4199230 | 349456.3 | 1932 | 0 | Human retinoic acid receptor (gamma-7) mRNA. |
| 1256 | 761060 | g2040531 | 332678.2 | 1872 | 0 | Human chromatin assembly factor-I p60 subunit mRNA, |
| | | | | | | complete cds. |
| 1257 | 678793 | g1882231 | | | | |
| 1258 | 873758 | g2199920 | | | | |
| 1259 | 482641 | g1555782 | 175142.1 | 1582 | 0 | |
| 1260 | 479758 | g1531348 | | | | |
| 1261 | 467472 | g1506720 | | | | |
| 1262 | 692981 | g1862437 | | | | |
| 1263 | 776036 | g1932193 | 239320.11 | 1747 | 0 | Human branched chain aminotransferase precursor (BCATm) |
| | | | | | | mRNA, nuclear gene encoding mitochondrial protein, |
| | | | | | | complete cds. |
| 1264 | 437831 | g1463987 | 214443.12 | 1641 | 0 | Human HSPC338 mRNA, partial cds. |
| 1265 | 579735 | g1671963 | 445101.5 | 2076 | 0 | Human cyclin protein gene, complete cds. |
| 1266 | 467550 | g1506490 | | | | |
| 1267 | 493305 | g1643728 | 480656.47 | 2126 | 0 | Human mRNA for stromal antigen 3 (STAG3 gene). |
| 1268 | 805386 | g2201432 | | | | |
| 1269 | 633625 | g1768163 | 429732.35 | 2061 | 0 | Human mitochondrial matrix protein P1 (nuclear encoded) mRNA, |
| | | | | | | complete cds. |
| 1270 | 426992 | g1446657 | 1188644.1 | 1542 | 0 | Human mRNA for kinesin-related protein, partial cds. |
| 1271 | 350848 | g1325096 | 232795.18c | 1673 | 0 | Human polyhomeotic 1 homolog (HPH1) mRNA, partial cds. |
| 1272 | 657961 | g1793977 | 200541.2 | 1611 | 0 | Human mRNA for putative transcription factor, partial. |
| 1273 | 961083 | g2308499 | | | | |
| 1274 | 481381 | g1554180 | 481594.12 | 2135 | 0 | Human cytosolic aspartate aminotransferase mRNA, |
| | | | | | | complete cds. |
| 1275 | 834225 | g2071749 | 362675.1 | 1966 | 0 | |
| 1276 | 407480 | g1542266 | 154551.8 | 1572 | 2.00E−07 | tex292 |
| 1277 | 422953 | g1427888 | | | | |
| 1278 | 576166 | g1682440 | | | | |
| 1279 | 403005 | g1539226 | 150712.6 | 1571 | 0 | Human mRNA for putative ATP(GTP)-binding protein, |
| | | | | | | partial. |
| 1280 | 355755 | g1332827 | 429649.5 | 2058 | 0 | Human ribosomal protein S6 kinase 1 (RPS6KA1) mRNA, |
| | | | | | | complete cds. |
| 1281 | 466382 | g1504972 | 815574.1c | 2177 | 0 | Human regulatory myosin light chain (MYL5) mRNA, |
| | | | | | | complete cds. |
| 1282 | 466384 | g4056994 | | | | |
| 1283 | 424705 | g1436780 | 1071715.6 | 1426 | 0 | Human KNSL4 and MAZ genes for kinesin-like DNA binding |
| | | | | | | protein and Myc-associated zinc finger protein, |
| | | | | | | complete cds. |
| 1284 | 574491 | g4058034 | | | | |
| 1285 | 696769 | g1864669 | 1086066.1 | 1482 | 0 | Human ribosomal protein S5 mRNA, complete cds. |
| 1286 | 477828 | g1528290 | 221749.3 | 1649 | 0.0007 | RS21-C6 |
| 1287 | 805046 | g2050228 | | | | |
| 1288 | 441327 | g1465322 | 1085840.2 | 1481 | 0 | Human RNA polymerase III subunit (RPC32) mRNA, |
| | | | | | | complete cds. |
| 1289 | 331792 | g1282100 | 336961.1 | 1888 | 0 | Human growth arrest specific 11 (GAS11) mRNA, |
| | | | | | | complete cds. |
| 1290 | 420278 | g4275596 | 899124.11 | 2195 | 0 | Human mRNA for mitochondrial DNA polymerase gamma, |
| | | | | | | complete cds. |
| 1291 | 426239 | g4271934 | 198198.2c | 1597 | 0 | Human Cctg mRNA for chaperonin. |
| 1292 | 466241 | g1504958 | 474444.2 | 2099 | 0 | Human poly(ADP-ribose) polymerase mRNA, complete cds. |
| 1293 | 917265 | g2222496 | | | | |
| 1294 | 337748 | g1309637 | 812158.1 | 2166 | 0 | Human asparagine synthetase mRNA, complete cds. |
| 1295 | 577488 | g1701407 | 474711.5 | 2105 | 0.001 | RPGR protein |
| 1296 | 465804 | g1504222 | 399899.2 | 1992 | 0 | Human mitotic checkpoint kinase Mad3L (MAD3L) mRNA, |
| | | | | | | complete cds. |
| 1297 | 406031 | g1540518 | 236330.2 | 1728 | 0 | Human mRNA for NAD-dependent methylene tetrahydrofolate |
| | | | | | | dehydrogenase cyclohydrolase (EC 1.5.1.15). |
| 1298 | 738252 | g1909743 | | | | |
| 1299 | 400035 | g1387154 | 474194.11 | 2091 | 0 | Human immunophilin (FKBP52) mRNA, complete cds. |
| 1300 | 791570 | g2142370 | 239001.1 | 1746 | 0 | |
| 1301 | 574520 | g1684335 | | | | |
| 1302 | 803622 | g2101604 | | | | |
| 1303 | 438886 | g4061634 | 1071715.6 | 1426 | 0 | Human KNSL4 and MAZ genes for kinesin-like DNA binding |
| | | | | | | protein and Myc-associated zinc finger protein, |
| | | | | | | complete cds. |
| 1304 | 734665 | g1895550 | | | | |
| 1305 | 776133 | g1918740 | 427922.1 | 2054 | 0 | |
| 1306 | 790721 | g2041524 | 203263.1 | 1630 | 0 | sarcosine oxidase |
| 1307 | 464084 | g1494430 | 196661.2 | 1586 | 0 | Human HSPC145 mRNA, complete cds. |
| 1308 | 791719 | g2074809 | 199439.1 | 1604 | 0 | Human selenophosphate synthetase 2 (SPS2) mRNA, |
| | | | | | | complete cds. |
| 1309 | 722631 | g4764843 | | | | |
| 1310 | 805579 | g4320452 | 317119.1 | 1847 | 0 | |
| 1311 | 571367 | g1655110 | | | | |
| 1312 | 575434 | g1684086 | | | | |
| 1313 | 793218 | g4320660 | | | | |
| 1314 | 525119 | g5598244 | 174035.1 | 1578 | 5.90E−42 | cystathionine gamma-lyase |
| 1315 | 441229 | g1472887 | 395096.3 | 1983 | 0 | Human mRNA for DNA replication licensing factor (huMCM2), |
| | | | | | | complete cds. |
| 1316 | 444844 | g1478979 | 809348.1 | 2165 | 0 | Human colon Kruppel-like factor (CKLF) mRNA, |
| | | | | | | complete cds. |
| 1317 | 463388 | g1497812 | | | | |
| 1318 | 533085 | g1576043 | 232772.6 | 1672 | 0 | Human mRNA for KIAA0116 gene, partial cds. |
| 1319 | 402730 | g1391116 | 245863.6 | 1777 | 0 | Human pyrroline 5-carboxylate reductase isoform (P5CR2) mRNA, |
| | | | | | | complete cds. |
| 1320 | 466047 | g1501086 | 331403.12 | 1857 | 0 | Human P1-Cdc46 mRNA. |
| 1321 | 641507 | g1796677 | 029777.3 | 1388 | 0 | Human beta-mannosidase mRNA, complete cds. |
| 1322 | 945359 | g2307964 | 251562.5 | 1794 | 0 | Human mutator gene (hMSH2) mRNA, complete cds. |
| 1323 | 765272 | g1913230 | | | | |
| 1324 | 456342 | g1487245 | | | | |
| 1325 | 314146 | g4258079 | 229683.19 | 1661 | 0 | Human histone acetyltransferase 1 mRNA, complete cds. |
| 1326 | 316914 | g1286251 | 232662.7 | 1671 | 0 | Human clone 25116 mRNA sequence. |
| 1327 | 779343 | g2074241 | 344520.3 | 1905 | 0 | Human glutamine PRPP amidotransferase (GPAT) mRNA, |
| | | | | | | complete cds. |
| 1328 | 582668 | g4284644 | 332649.7 | 1870 | 0 | Human LPAP mRNA for lysophosphatidic acid phosphatase, |
| | | | | | | complete cds. |
| 1329 | 313254 | g1286209 | 233307.2 | 1684 | 8.00E−77 | ubiquitin-specific processing protease |
| 1330 | 945218 | g2306681 | 337822.11 | 1892 | 0 | Human mRNA for M96A protein. |
| 1331 | 403663 | g1539711 | | | | |
| 1332 | 833511 | g2192127 | 344297.4 | 1904 | 0 | Human cyclin mRNA. |
| 1333 | 748744 | g1934363 | 1189351.1 | 1548 | 0 | Human mRNA for transcription elongation factor TFIIS.h. |
| 1334 | 933491 | g2288782 | 757504.15 | 2141 | 0 | Human mRNA for nucleolar protein hNop56. |
| 1335 | 575290 | g1684581 | | | | |
| 1336 | 751977 | g2049363 | 286623.1 | 1838 | 5.00E−81 | Human cyclin B mRNA, 3′ end. |
| 1337 | 572970 | g1662435 | 330872.4 | 1851 | 0 | Human replication factor C, 36-kDa subunit mRNA, |
| | | | | | | complete cds. |
| 1338 | 572379 | g1659837 | 475213.1 | 2111 | 0 | Human mRNA for very-long-chain acyl-CoA synthetase, |
| | | | | | | complete cds. |
| 1339 | 476629 | g1530212 | 1185387.1 | 1529 | 0 | Human mRNA for GARS-AIRS-GART. |
| 1340 | 477593 | g1528231 | 1170290.3c | 1518 | 0 | Human mRNA for GARS-AIRS-GART. |
| 1341 | 920268 | g2284236 | 427797.38c | 2053 | 0 | Human hmgI mRNA for high mobility group protein I. |
| 1342 | 805549 | g2050293 | 235420.3 | 1720 | 0 | Human equilibrative nucleoside transporter 1 (ENT1) gene, |
| | | | | | | complete cds. |
| 1343 | 680512 | g4306841 | 235420.3 | 1720 | 0 | Human equilibrative nucleoside transporter 1 (ENT1) gene, |
| | | | | | | complete cds. |
| 1344 | 403735 | g1539760 | 029997.1 | 1389 | 0 | |
| 1345 | 693719 | g1901373 | 332649.4 | 1869 | 0 | Human LPAP mRNA for lysophosphatidic acid phosphatase, |
| | | | | | | complete cds. |
| 1346 | 638455 | g1826018 | 411296.5 | 2038 | 0 | Human hemopoietic cell protein-tyrosine kinase (HCK) gene, |
| | | | | | | complete cds, clone lambda-a2/1a. |
| 1347 | 579391 | g1680969 | | | | |
| 1348 | 402162 | g1387914 | | | | |
| 1349 | 833919 | g2071845 | 366398.4 | 1968 | 0 | Human mRNA for JM1 protein, complete cds (clone |
| | | | | | | LLNLc110M0111Q7 (RZPD Berlin) and LLNLc110K2140Q7 |
| | | | | | | (RZPD Berlin)). |
| 1350 | 776058 | g4318153 | | | | |
| 1351 | 367627 | g1355503 | | | | |
| 1352 | 457955 | g4058612 | | | | |
| 1353 | 949512 | g2283100 | 349423.5 | 1931 | 0 | Human asialoglycoprotein receptor H2 mRNA, complete cds. |
| 1354 | 334182 | g1537449 | 244606.5 | 1766 | 0 | Human genomic DNA, chromosome 22q11.2, clone N27C7. |
| 1355 | 893933 | g2256530 | | | | |
| 1356 | 820336 | g2102082 | | | | |
| 1357 | 442396 | g1474858 | 1184163.1c | 1527 | 0 | Human mRNA for KIAA0888 protein, partial cds. |
| 1358 | 442966 | g1476058 | | | | |
| 1359 | 833557 | g2116381 | 757572.1 | 2144 | 0 | Human mRNA for B-myb gene. |
| 1360 | 791079 | g2075275 | 017194.2 | 1376 | 0 | |
| 1361 | 833477 | g2192112 | 401524.1 | 2001 | 5.00E−43 | F-box protein FBX15 |
| 1362 | 445862 | g1483937 | 470468.6 | 2088 | 0 | Human mRNA for uridine phosphorylase. |
| 1363 | 535794 | g1656688 | | | | |
| 1364 | 633484 | g1768070 | | | | |
| 1365 | 944963 | g2283154 | 203237.1 | 1629 | 0 | Human jun dimerization protein gene, partial cds; |
| | | | | | | cfos gene, complete cds; and unknown gene. |

What is claimed is:

1. A combination comprising a plurality of isolated cDNAs, wherein the cDNAs are SEQ ID NOs: 1-2217 that are differentially expressed in neuronal differentiation and morphogenesis or the complements of SEQ ID NOs: 1-2217.

2. The combination of claim 1 wherein the cDNAs are SEQ ID NOs: 1-1365 that are differentially expressed during neuronal differentiation and morphogenesis or the complements of SEQ ID NOs: 1-1365.

3. The combination of claim 1, wherein the cDNAs are SEQ ID NOs: 1366-2217 or the complements of SEQ ID NOs: 1366-2217.

4. The combination of claim 1, wherein each cDNA is homologous to a cDNA that was differentially expressed greater than 5-fold and is selected from
a) SEQ ID NOs: 1366, 1367, 1369, 1379, 1396, 1417, 1424, 1429, 1434, 1450, 1468, 1470, 1475, 1487, 1492, 1497, 1508, 1512, 1522, 1533, 1552, 1560, 1596, 1607, 1614, 1622, 1635, 1653, 1654, 1660, 1660, 1662, 1678, 1679, 1680, 1710, 1713, 1716, 1717, 1770, 1771, 1795, 1814, 1815, 1816, 1817, 1819, 1831, 1834, 1837, 1878, 1880, 1887, 1916, 1927, 1948, 1950, 1976, 2019, 2022, 2039, 2041, 2060, 2060, 2063, 2084, 2127, 2127, 2154, 2164, 2182, 2183;
b) SEQ ID NOs: 1376, 1388, 1389, 1426, 1518, 1527, 1529, 1548, 1578, 1586, 1604, 1629, 1630, 1661, 1671, 1672, 1684, 1720, 1766, 1777, 1794, 1838, 1847, 1851, 1857, 1869, 1870, 1892, 1904, 1905, 1931, 1968, 1983, 2001, 2038, 2053, 2054, 2088, 2111, 2141, 2144, 2165; and
c) a complement of the cDNAs of a) and b).

5. The combination of claim 1, wherein each cDNA is differentially expressed greater than 10-fold and is selected from
a) SEQ ID NOs: 1379, 1607, 1653, 1654, 1678, 1679, 1713, 1771, 1976, 2022, 2127, 2164;
b) SEQ ID NOs: 1376, 1527, 1629, 1766, 1931, 2001, 2088, 2144; and
c) a complement of the cDNAs of a) and b).

6. A method of using a combination to detect expression of a nucleic acid in a sample comprising:
a) hybridizing the combination of claim 1 to a sample under conditions for formation of one or more hybridization complexes;
b) detecting hybridization complex formation, wherein complex formation indicates expression of at least one nucleic acid in the sample.

7. The method of claim 6 wherein the combination is attached to a substrate.

8. The method of claim 6 wherein the sample is from adult neuronal or embryonic stem cells.

9. The method of claim 3 wherein expression indicates the presence of a disorder associated with neuronal differentiation and morphogenesis.

10. A method of screening a plurality of molecules or compounds to identify at least one molecule or compound which specifically binds a cDNA of the combination comprising:
a) contacting the combination of claim 1 with a plurality of molecules or compounds under conditions to allow specific binding; and
b) detecting specific binding, thereby identifying a molecule or compound which specifically binds a cDNA of the combination.

11. The method of claim 7 wherein the molecules and compounds to be screened are selected from DNA molecules, RNA molecules, peptide nucleic acids, transcription factors, enhancers, repressors, mimetics, and proteins.

12. An isolated cDNA selected from SEQ ID NOs: 1367, 1372, 1376, 1377, 1383, 1389, 1390, 1397, 1400, 1401, 1406, 1408, 1412, 1413, 1415, 1416, 1420, 1438, 1444, 1460, 1462, 1467, 1470, 1476, 1477, 1485, 1489, 1491, 1497, 1500, 1501, 1503, 1512, 1517, 1541, 1544, 1551, 1553, 1555, 1557, 1565, 1566, 1572, 1576, 1577, 1579, 1580, 1581, 1582, 1583, 1585, 1588, 1598, 1599, 1601, 1603, 1609, 1610, 1612, 1620, 1625, 1632, 1634, 1642, 1643, 1649, 1658, 1686, 1690, 1691, 1693, 1708, 1721, 1727, 1730, 1731, 1738, 1746, 1748, 1756, 1757, 1786, 1789, 1797, 1823, 1825, 1829, 1830, 1847, 1852, 1864, 1866, 1875, 1882, 1887, 1889, 1894, 1895, 1901, 1938, 1949, 1954, 1960, 1961, 1965, 1966, 2002, 2019, 2043, 2046, 2054, 2066, 2067, 2069, 2071, 2072, 2078, 2079, 2100, 2105, 2121, 2125, 2139, 2151, 2154, 2157, 2170, 2171, 2183, 2184, 2186, 2187, 2208, 2211, and the complements thereof

13. An expression vector containing a cDNA of claim 12.

14. A host cell containing the expression vector of claim 13.

15. A method for producing a protein, the method comprising the steps of:
(a) culturing the host cell of claim 14 under conditions for the expression of protein; and
(b) recovering the protein from the host cell culture.

16. A protein produced by the method of claim 14.

17. A method for screening a plurality of molecules or compounds to identify at least one ligand which specifically binds a protein, the method comprising:
a) combining the protein of claim 15 with the library under conditions to allow specific binding; and
b) detecting specific binding between the protein and a molecule or compound, thereby identifying a ligand which specifically binds the protein.

18. The method of claim 16 wherein the plurality of molecules and compounds are selected from DNA molecules, RNA molecules, peptide nucleic acids, mimetics, peptides, proteins, agonists, antagonists, antibodies or their fragments, immunoglobulins, inhibitors, drug compounds, and pharmaceutical agents.

18. A purified antibody which specifically binds the protein of claim 16.

19. An agonist which specifically bind the protein of claim 16.

20. An antagonist which specifically binds the protein of claim 16.
